(19) United States
(12) Patent Application Publication
(10) Pub. No.: US 2011/0117551 A1
(43) Pub. Date: May 19, 2011

(54) DETECTION AND PROGNOSIS OF LUNG CANCER

(75) Inventors: Wim Van Criekinge, Sart-Tilman (BE); Josef Straub, Sart-Tilman (BE); Geert Trooskens, Sart-Tilman (BE); Stephen Baylin, Baltimore, MD (US); James Herman, Baltimore, MD (US); Kornel Schuebel, Baltimore, MD (US); Leslie Cope, Baltimore, MD (US); Leander Van Neste, Gent (BE)

(73) Assignees: ONCOMETHYLOME SCIENCES SA, Sart-Tilman (Liege) (BE); THE JOHNS HOPKINS UNIVERSITY, Baltimore, MD (US)

(21) Appl. No.: 12/867,539

(22) PCT Filed: Feb. 19, 2009
(86) PCT No.: PCT/US2009/034531

§ 371 Date: Dec. 22, 2010

Related U.S. Application Data
(60) Provisional application No. 61/029,693, filed on Feb. 19, 2008.

(51) Int. Cl. C12Q 1/68; G01N 33/574

(52) U.S. Cl. 435/6; 435/7.23

(57) ABSTRACT

Methods and tools are provided for detecting and predicting lung cancer. The methods and tools are based on epigenetic modification due to methylation of genes in lung cancer or pre-lung cancer. The tools can be assembled into kits or can be used seperately. Genes found to be epigentically silenced in association with lung cancer include ACSL6, ALS2CL, APC2, ART-S1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655.

### TECHNICAL FIELD OF THE INVENTION

The present invention relates to the area of cancer diagnostics and therapeutics. In particular, it relates to methods and kits for identifying, diagnosing, prognosing and monitoring lung cancer. These methods include determining the methylation status or the expression levels of particular genes, or a combination thereof. In particular, the lung cancer relates to non-small cell lung cancer.

### BACKGROUND OF THE INVENTION

Lung cancer is the most common cause of cancer-related death and causes over one million deaths worldwide each year (Greenlee et al, 2001). Lung cancer is clinically subdivided into small cell lung cancer (SCLC; comprise about 20% of lung cancers), the most aggressive form of lung cancer, and non-small cell lung cancer (NSCLC, the most common lung cancer accounting for about 80%), consisting of adenocarcinoma, squamous cell carcinoma, large cell carcinoma, and miscellaneous other types such as carcinoids, pleomorphic and mixed carcinomas and a range of neuroendocrine cancers (Travis, 2002).

The first signs of cancer usually come from one or more of the following sources: presentation of symptoms, visual detection or direct palpation, histopathological analysis of a biopsy specimen, remote imaging or the detection of a cancer biomarker in a tissue or bodily fluid specimen. The rather late appearance of symptomatology associated with lung cancer, and the poor accessibility to the lung tissue thwart the timely detection of malignancy, contributing to high mortality rates (Ganti et al., 2006; Greenberg et al., 2007). Therefore, remote imaging and the development of cancer biomarkers offers the best hope for early detection of lung cancer.

Cancer biomarkers have been described in literature. One can distinguish between immunological markers and genetic markers. Genetic markers are based on detection of mutation in distinct genes in particular in tumor suppressor genes. More recently, DNA methylation markers are evaluated as potential genetic markers for detection of cancer because they offer certain advantages when compared to mutation markers. One of the most important features is that they occur at the early stages of cancer development and in many cases are tissue- and tumor-type specific (Esteller et al. 2001). A further advantage, methylation profile is preserved in purified isolated DNA and methylation changes appear to precede apparent malignancy in many cases. In addition, methylation markers may serve for predictive purposes as they often reflect the sensitivity to therapy or duration of patient survival.

DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. See, J. G. Herman, Seminars in Cancer Biology, 9: 359-67, 1999. By turning genes off that are not needed, DNA methylation is an essential control mechanism for the normal development and functioning of organisms. Alternatively, abnormal DNA methylation is one of the mechanisms underlying the changes observed with aging and development of many cancers.

Although the phenomenon of gene methylation has attracted the attention of cancer researchers for some time, its true role in the progression of human cancers is just now being recognized. In normal cells, methylation occurs predominantly in regions of DNA that have few CG base repeats, while CpG islands, regions of DNA that have long repeats of CG bases, remain non-methylated. Gene promoter regions that control protein expression are often CpG island-rich. Aberrant methylation of these normally non-methylated CpG islands in the promoter region causes transcriptional inactivation or silencing of certain tumor suppressor expression in human cancers.

Genes that are hypermethylated in tumor cells are strongly specific to the tissue of origin of the tumor. Molecular signatures of cancers of all types can be used to improve cancer detection, the assessment of cancer risk and response to therapy. Promoter hypermethylation events provide some of the most promising markers for such purposes.

An early diagnosis is critical for the successful treatment of many types of cancer, including lung cancer. If the exact methylation profiles of lung tumors are available and drugs targeting the specific genes are obtainable, then the treatment of lung cancer could be more focused and rational. Therefore, the detection and mapping of novel methylation markers is an essential step towards improvement of lung cancer prevention, screening and treatment.

There is a continuing need in the art to identify methylation markers that can be used for improved assessment of lung cancer.

### SUMMARY OF THE INVENTION

According to one embodiment of the invention a method is provided for identifying lung cancer or its precursor, or predisposition to lung cancer. Epigenetic modification of at least one gene selected from the group consisting of ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655 is detected in a test sample containing lung cells or nucleic acids from lung cells. The test sample is identified as containing cells that are neoplastic, precursor to neoplastic, or predisposed to neoplasia, or as containing nucleic acids from cells that are neoplastic, precursor to neoplastic, or predisposed to neoplasia.

According to another embodiment of the invention a kit is provided for assessing lung cancer or its precursor, or predisposition to lung cancer in a test sample containing lung cells or nucleic acids from lung cells. The kit comprises in a package: a reagent that (a) modifies methylated cytosine residues but not non-methylated cytosine residues, or that (b) modifies non-methylated cytosine residues but not methylated cytosine residues; and at least one pair of oligonucleotide primers that specifically hybridizes under amplification conditions to a region of a gene selected from the group consisting of ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655. The region is within about 3 kb of said gene's transcription start site.

Another embodiment of the invention provides a second kit for assessing lung cancer or its precursor, or predisposition to lung cancer in a test sample containing lung cells or nucleic acids from lung cells. The kit comprises in a package: at least two pairs of oligonucleotide primers that specifically hybridize under amplification conditions to a region of a gene selected from the group consisting of ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655. The region is within about 3 kb of said gene's transcription start site.

An additional aspect of the invention provides an isolated polynucleotide. The polynucleotide comprises a nucleotide sequence selected from the group consisting of SEQ ID NO: 1-449.

These and other embodiments which will be apparent to those of skill in the art upon reading the specification provide the art with reagents and methods for detecting lung cancer, early lung cancer, or predisposition to lung cancer.

### BRIEF DESCRIPTION OF THE DRAWINGS

FIG. 1: Position of the different primers relative to the TSS (transcription start site). Multiple primer designs are displayed by blue boxes and red boxes (=final primer pairs retained for the assays). The exon of FBN2 is indicated in green. The number of CpG count is spotted in blue over a region of 20 kb.

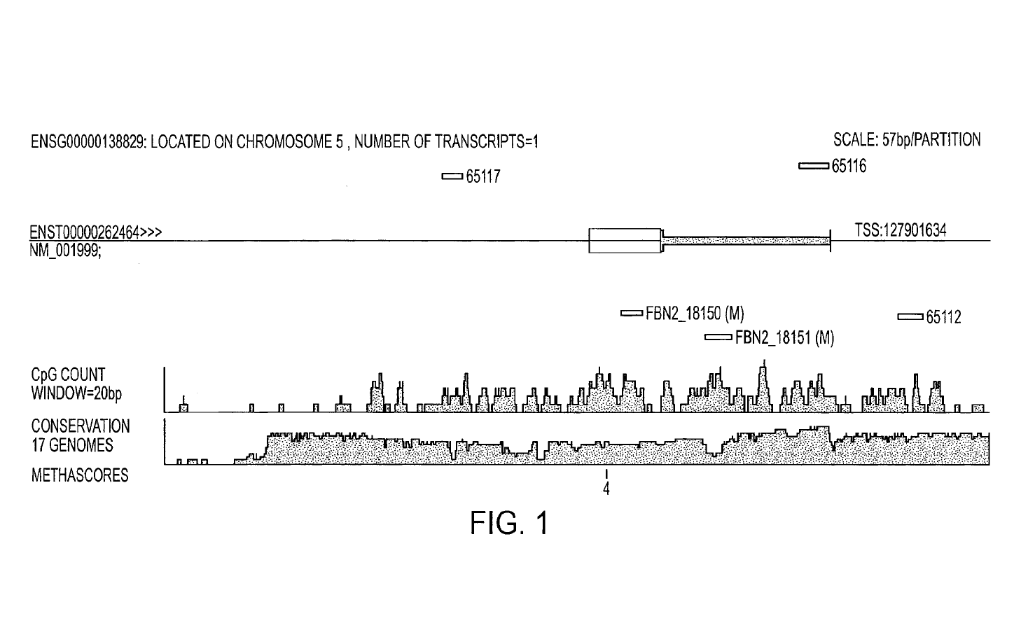

FIG. 2 lists the sequences of the different primer sets and converted and unconverted amplicon sequences used in FIG. 1.

FIG. 3: Ranked methylation table obtained with the sample set. 146 methylation profiles from lung cancer samples (right table) are compared against 58 normal tissue samples (left table). Samples are shown along the Y-axis where each horizontal row represents the methylation profile of one individual sample across the 23 different assays (X-axis). Assays demonstrating the best methylation discriminators between the 2 groups are displayed at the left, with discrimination effect decreasing towards the right. The black boxes indicate the methylated results; grey boxes indicate the unmethylated results; white boxes indicate invalid results.

FIG. 4: Amplification plot for the standard curve for JAM3

FIG. 5: Amplification plot for standard curve and samples for JAM3

FIG. 6: Linear regression of standard curve for JAM3

FIG. 7: Decision tree for ratio determination

FIG. 8: Performance of the individual markers on lung tissue samples using qMSP.

### DETAILED DESCRIPTION OF THE INVENTION

The inventors have found that cytosines within CpG dinucleotides of DNA from particular genes isolated from a test sample are differentially methylated in human lung cancer tissue samples and normal lung tissue control samples. The cancer tissues samples are hypermethylated or hypomethylated with respect to the normal samples (collectively termed epigenetic modification). The differential methylation has been found in genomic DNA of ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655. These genes are all known in the art and fully described by sequence in publicly available databases, e.g., Entrez Gene of the National Center for Biotechnology Information. See Gene ID references provided in Table 1 and Table 3, each of which is incorporated by reference herein.

Epigenetic modification of a gene can be determined by any method known in the art.

One method is to determine that a gene which is expressed in normal cells or other control cells is less expressed or not expressed in tumor cells. Conversely, a gene can be more highly expressed in tumor cells than in control cells in the case of hypomethylation. This method does not, on its own, however, indicate that the silencing or activation is epigenetic, as the mechanism of the silencing or activation could be genetic, for example, by somatic mutation. One method to determine that silencing is epigenetic is to treat with a reagent, such as DAC (5′-deazacytidine), or with a reagent which changes the histone acetylation status of cellular DNA or any other treatment affecting epigenetic mechanisms present in cells, and observe that the silencing is reversed, i.e., that the expression of the gene is reactivated or restored. Another means to determine epigenetic modification is to determine the presence of methylated CpG dinucleotide motifs in the silenced gene or the absence of methylation CpG dinucleotide motifs in the activated gene. Typically these methylated motifs reside near the transcription start site, for example, within about 3 kbp, within about 2.5 kbp, within about 2 kbp, within about 1.5 kbp, within about 1 kbp, within about 750 bp, or within about 500 bp. CpG dinucleotides susceptible to methylation are typically concentrated in the promoter region, intron region or exon region of human genes. Thus, the methylation status of the promoter and/or intron and/or exon region of at least one gene can be assessed. Once a gene has been identified as the target of epigenetic modification in tumor cells, determination of reduced or enhanced expression can be used as an indicator of epigenetic modification.

Expression of a gene can be assessed using any means known in the art. Typically expression is assessed and compared in test samples and control samples which may be normal, non-malignant cells. The test samples may contain cancer cells or pre-cancer cells or nucleic acids from them. For example the sample may contain lung adenoma cells, lung advanced adenoma cells, or lung adenocarcinoma cells. Samples may contain squamous cells, and large cell carcinoma. Samples may contain mixtures of different types and stages of lung cancer cells. Either mRNA (nucleic acids) or protein can be measured to detect epigenetic modification. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. In some embodiments oligonucleotide probes are covalently linked to primers for amplification. Advances in genomic technologies now permit the simultaneous analysis of thousands of genes, although many are based on the same concept of specific probe-target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Specific proteins can be assessed using any convenient method including immunoassays and immunocytochemistry but are not limited to that. Most such methods will employ antibodies which are specific for the particular protein or protein fragments. The sequences of the mRNA (cDNA) and proteins of the markers of the present invention are known in the art and publicly available.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa II, and Not I. Alternatively, chemical reagents can be used which selectively modify either the methylated or non-methylated form of CpG dinucleotide motifs.

Modified products can be detected directly, or after a further reaction which creates products which are easily distinguishable. Means which detect altered size and/or charge can be used to detect modified products, including but not limited to electrophoresis, chromatography, and mass spectrometry. Examples of such chemical reagents for selective modification include hydrazine and bisulfite ions. Hydrazine-modified DNA can be treated with piperidine to cleave it. Bisulfite ion-treated DNA can be treated with alkali. Other means which are reliant on specific sequences can be used, including but not limited to hybridization, amplification, sequencing, and ligase chain reaction, Combinations of such techniques can be uses as is desired.

The principle behind electrophoresis is the separation of nucleic acids via their size and charge. Many assays exist for detecting methylation and most rely on determining the presence or absence of a specific nucleic acid product. Gel electrophoresis is commonly used in a laboratory for this purpose.

One may use MALDI mass spectrometry in combination with a methylation detection assay to observe the size of a nucleic acid product. The principle behind mass spectrometry is the ionizing of nucleic acids and separating them according to their mass to charge ratio. Similar to electrophoresis, one can use mass spectrometry to detect a specific nucleic acid that was created in an experiment to determine methylation. See (Tost, J. et al. 2003).

One form of chromatography, high performance liquid chromatography, is used to separate components of a mixture based on a variety of chemical interactions between a substance being analyzed and a chromatography column. DNA is first treated with sodium bisulfite, which converts an unmethylated cytosine to uracil, while methylated cytosine residues remain unaffected. One may amplify the region containing potential methylation sites via PCR and separate the products via denaturing high performance liquid chromatography (DHPLC). DHPLC has the resolution capabilities to distinguish between methylated (containing cytosine) and unmethylated (containing uracil) DNA sequences. (Deng, D. et al. 2002)

Hybridization is a technique for detecting specific nucleic acid sequences that is based on the annealing of two complementary nucleic acid strands to form a double-stranded molecule. One example of the use of hybridization is a microarray assay to determine the methylation status of DNA. After sodium bisulfite treatment of DNA, which converts an unmethylated cytosine to uracil while methylated cytosine residues remain unaffected, oligonucleotides complementary to potential methylation sites can hybridize to the bisulfite-treated DNA. The oligonucleotides are designed to be complimentary to either sequence containing uracil (thymine) or sequence containing cytosine, representing unmethylated and methylated DNA, respectively. Computer-based microarray technology can determine which oligonucleotides hybridize with the DNA sequence and one can deduce the methylation status of the DNA. Similarly primers can be designed to be complimentary to either sequence containing uracil (thymine) or sequence containing cytosine. Primers and probes that recognize the converted methylated form of DNA are dubbed methylation-specific primers or probes (MSP).

An additional method of determining the results after sodium bisulfite treatment involves sequencing the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR) (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), microsatellite length polymorphism (MLP), and nick displacement amplification (WO2004/067726).

Sequence variation that reflects the methylation status at CpG dinucleotides in the original genomic DNA offers two approaches to PCR primer design. In the first approach, the primers do not themselves “cover” or hybridize to any potential sites of DNA methylation; sequence variation at sites of differential methylation are located between the two primers. Such primers are used in bisulfite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfate ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

In one particular embodiment, primers useful in MSP carried out on the gene selected from ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655 are provided. Primers of the invention preferably are designed to amplify the genomic sequences in the regions under investigation. Preferred regions may comprise, consist essentially of or consist of the sequences represented by SEQ ID NO. 129-192 and/or SEQ ID NO. 193-256 and/or SEQ ID NO. 315-329 and/or SEQ ID NO. 330-344 and/or SEQ ID NO. 408-428 and/or SEQ ID NO. 429-449 and/or SEQ ID NO. 271-277 and/or SEQ ID NO. 278-284. Preferred sense primers (5′-3′) may comprise, consist essentially of or consist of the sequences represented by SEQ ID NO. 1-64 and/or SEQ ID NO. 285-299 and/or SEQ ID NO. 345-365 and/or SEQ ID NO. 257-263. Preferred antisense primers (5′-3′) comprise, consist essentially of or consist of the sequences represented by SEQ ID NO. 65-128 and/or SEQ ID NO. 300-314 and/or SEQ ID NO. 366-386 and/or SEQ ID NO. 264-270.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

In one particular embodiment, probes useful in MSP carried out on the gene selected from ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655 are provided. Probes of the invention preferably are designed to bind to genomic sequences in the regions under investigation. Preferred regions may comprise, consist essentially of or consist of the sequences represented by SEQ ID NO. 129-192 and/or SEQ ID NO. 193-256 and/or SEQ ID NO. 315-329 and/or SEQ ID NO. 330-344 and/or SEQ ID NO. 408-428 and/or SEQ ID NO. 429-449 and/or SEQ ID NO. 271-277 and/or SEQ ID NO. 278-284. Preferred probes (5′-3′) may comprise, consist essentially of or consist of the sequences represented by SEQ ID NO. 387-407.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction enconuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TaqMan™ (Roche Molecular Systems) system and Molecular Beacon™ system which have separate probes labeled with a fluorophore and a fuorescence quencher. In the Scorpion™ system the labeled probe in the form of a hairpin structure is linked to the primer. In addition, the Amplifluor™ (Chemicon International) system and the Plexor™ (Promega) system can be used.

DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleotide-based microarray systems.

Subsets of genes for all aspects and embodiments of the invention include ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655. By “gene” is meant any gene which is taken from the family to which the named “gene” belongs and includes according to all aspects of the invention not only the particular sequences found in the publicly available database entries, but also encompasses transcript and nucleotide variants of these sequences, with the proviso that methylation or another epigenetic modification of the gene is linked to lung cancer. The number of genes whose modification is tested and/or detected can vary: one, two, three, four, five, or more genes can be tested and/or detected. In some cases at least two genes are selected. In other embodiments at least three genes are selected.

Testing can be performed diagnostically or in conjunction with a therapeutic regimen.

Testing can be used to monitor efficacy of a therapeutic regimen, whether a chemotherapeutic agent or a biological agent, such as a polynucleotide. Testing can also be used to determine what therapeutic or preventive regimen to employ on a patient. Moreover, testing can be used to stratify patients into groups for testing agents and determining their efficacy on various groups of patients. The detection may also link to a cancer stage or grade. The “Stage” refers to how far a cancer has progressed anatomically, while the “grade” refers to cell appearance (differentiation) and DNA make up.

Test samples and normal samples for diagnostic, prognostic, or personalized medicine uses can be obtained from surgical samples, such as biopsies or fine needle aspirates, from paraffin embedded lung, or other organ tissues, from a body fluid such as blood, serum, lymph, saliva, sputum, urine, pleural fluid, bronchoalveolar lavage fluid. Such sources are not meant to be exhaustive, but rather exemplary. A test sample obtainable from such specimens or fluids includes detached tumor cells and/or free nucleic acids that are released from dead or damaged tumor cells. Nucleic acids include RNA, genomic DNA, mitochondrial DNA, single or double stranded, and protein-associated nucleic acids. Any nucleic acid specimen in purified or non-purified form obtained from such specimen cell can be utilized as the starting nucleic acid or acids. The test samples may contain cancer cells or pre-cancer cells or nucleic acids from them. For example the sample may contain lung adenoma cells, lung advanced adenoma cells, or lung adenocarcinoma cells. Samples may contain squamous cells or large cell carcinoma. Samples may contain mixtures of different types and stages of lung cancer cells.

The test sample is generally obtained from a (human) subject suspected of being tumorigenic. Alternatively the test sample is obtained from a subject undergoing routine examination and not necessarily being suspected of having a disease. Thus patients at risk can be identified before the disease has a chance to manifest itself in terms of symptoms identifiable in the patient. Alternatively the sample is obtained from a subject undergoing treatment, or from patients being checked for recurrence of disease.

Demethylating agents can be contacted with cells in vitro or in vivo for the purpose of restoring normal gene expression to the cell. Suitable demethylating agents include, but are not limited to 5-aza-2′-deoxycytidine, 5-aza-cytidine, Zebularine, procaine, and L-ethionine. This reaction may be used for diagnosis, for determining predisposition, and for determining suitable therapeutic regimes.

Although diagnostic and prognostic accuracy and sensitivity may be achieved by using a combination of markers, such as 5 or 6 markers, or 9 or 10 markers, practical considerations may dictate use of smaller combinations. Any combination of markers for a specific cancer may be used which comprises 2, 3, 4, or 5 markers. Combinations of 2, 3, 4, or 5 markers can be readily envisioned given the specific disclosures of individual markers provided herein. Preferably, the invention involves detecting an epigenetic change in a panel of genes comprising a combination of 2, 3, 4 or 5 markers. Preferably, the panel comprises RASSF1A and/or SOX17 and/or HS3ST2-nor and/or NID2 and/or SFRP1.

Kits according to the present invention are assemblages of reagents for testing methylation. They are typically in a package which contains all elements, optionally including instructions. The package may be divided so that components are not mixed until desired. Components may be in different physical states. For example, some components may be lyophilized and some in aqueous solution. Some may be frozen. Individual components may be separately packaged within the kit. The kit may contain reagents, as described above for differentially modifying methylated and non-methylated cytosine residues. Desirably the kit will contain oligonucleotide primers which specifically hybridize to regions within 3 kb of the transcription start sites of the genes/markers: ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, and ZNF655. Additional markers may be used. Typically the kit will contain both a forward and a reverse primer for a single gene or marker. If there is a sufficient region of complementarity, e.g., 12, 15, 18, or 20 nucleotides, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues. The kit may optionally contain oligonucleotide probes. The probes may be specific for sequences containing modified methylated residues or for sequences containing non-methylated residues. The kit may optionally contain reagents for modifying methylated cytosine residues. The kit may also contain components for performing amplification, such as a DNA polymerase (particularly a thermostable DNA polymerase) and deoxyribonucleotides. Means of detection may also be provided in the kit, including detectable labels on primers or probes. Kits may also contain reagents for detecting gene expression for one of the markers of the present invention (Table 1 and Table 3). Such reagents may include probes, primers, or antibodies, for example. In the case of enzymes or ligands, substrates or binding partners may be sued to assess the presence of the marker. Kits may contain 1, 2, 3, 4, or more of the primers or primer pairs of the invention. Kits that contain probes may have them as separate molecules or covalently linked to a primer for amplifying the region to which the probes hybridize. Other useful tools for performing the methods of the invention or associated testing, therapy, or calibration may also be included in the kits, including buffers, enzymes, gels, plates, detectable labels, vessels, etc.

In one aspect of this embodiment, the gene is contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

Test compounds can be tested for their potential to treat cancer. Expression of a gene selected from those listed in Table 1 and Table 3 is determined and if it is increased or decreased by the compound in the cell or if methylation of the gene is decreased or increased by the compound in the cell, one can identify it as having potential as a treatment for cancer. The candidate compound will have the effect of reversing the expression/or methylation modification found in the cancer cell.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

### Example 1

### Selection of Candidate Genes

Using re-expression profiles of lung cancer cell lines, candidate genes were identified and the most promising markers were tested on tissue using the Base5 methylation profiling platform (Straub et al. 2007). Differential methylation of the particular genes was assessed using Base5 methylation profiling platform as follows: DNA was extracted from lung samples, bisulfite converted, and selected regions of the particular genes were amplified using primers whose sequence represented converted or non-converted DNA sequences. Amplification was monitored in real-time set up using cybergreen. Two robust data analyses designed to cope with inherent variance (i.e., noise) in measured Ct and Tm values were applied to withhold 64 different assays for detecting differential methylation of ACSL6, ALS2CL, APC2, BEX1, BMP7, CBR3, CD248, CD44, CHD5, DLK1, DPYSL4, DSC2, EPB41L3, EPHB6, ERBB3, FBLN2, FBN2, FOXL2, GSTP1, HS3ST2, IGFBP7, IRF7, JAM3, LOX, LY6D, LY6K, MACF1, MCAM, NEFH, NID2, PCDHB15, PHACTR3, POMC, PRKCA, PSEN1, RBP1, RRAD, SFRP1, SOD3, SOX17, SULF2, TIMP3, TJP2, TRPV2, UCHL1, WDR69, ZFP42, ZNF442, and ZNF655 in lung cancer tissue samples.

### Materials and Methods

### Strategy to Identify Supplementary Gene Targets for Lung Cancer

Promoter sequences were linked with gene expression to identify epigenetically silenced genes. An established pharmacologic unmasking strategy (5-aza-2′-deoxycytidine [DAC] and trichostatin A [TSA]) for re-expression analysis of epigenetically targeted genes was combined with proprietary advanced bioinformatics tools to identify genes prone to promoter methylation. To identify differentially methylated markers associated with non-small cell lung cancer (NSCLC), the information derived from 11 cell lines (ATCC, at domain lgcpromochem-atcc.com/, 2006) was used:

1. NCI-H23: adenocarcinoma, cell line derived from a smoker

2. NCI-H1568: adenocarcinoma, cell line derived from a smoker

3. NCI-H1993: adenocarcinoma, cell line derived from a smoker

4. NCI-H2023: adenocarcinoma, cell line derived from a non-smoker

5. NCI-H2085: adenocarcinoma, cell line derived from a non-smoker

6. NCI-H2228: adenocarcinoma, cell line derived from a non-smoker

7. NCI-H520: squamous cell carcinoma, cell line

8. NCI-H838: adenocarcinoma, cell line derived from a smoker

9. NCI-H2170: squamous cell carcinoma, cell line derived from a non-smoker

10. NCI-H1869: squamous cell carcinoma, cell line derived from a smoker

11. SK-MES-1: squamous cell carcinoma, cell line

Cell culture, microarray and data analysis was done as described in Schuebel et al, 2007. In short, the cell lines were cultured with 5-aza-2′-deoxycytidine (AZA) and with trichostatin A (TSA) in parallel. Control cells underwent mock treatment. Total RNA was harvested from AZA-, TSA- and mock-treated cells. Amplification and labeling of the RNA were carried out using the Low RNA Input Linear Amplification kit (Agilent Technologies). The complementary labeled RNA was hybridized and processed according the Agilent microarray protocol. All calculations and normalizations of the expression data were performed using the R statistical computing platform (Ihaka et al., 1996) and packages from Bioconductor bioinformatics software (Gentleman et al., 1996).

A gene was selected as a good candidate if it met the following criteria:

1. Re-expressed under AZA treatment; a gene was termed as a top tier gene if the expression is up regulated by more than two-fold in the AZA treated versus mock sample on the Agilent whole human genome expression microarray platform; if it showed an enrichment between 1.4 and 2 fold it was termed as a next tier gene

2. Silent, i.e., having no basal expression, in the mock cells

3. No response to TSA treatment alone

Following this initial candidate selection, 2 main strategies were taken to further select good gene candidates susceptible to hypomethylation and/or hypermethylation: a computational strategy and a verification strategy based on cell lines and primary tumors.

### Computational Strategy

This strategy was applied on the top and next tier genes of the first 6 cell lines (NCI-H23, NCI-H1568, NCI-H1993, NCI-H2023, NCI-H2085, and NCI-H2228).

Different steps were taken towards identification of good candidate genes susceptible to hypomethylation and/or hypermethylation:

Step 1: The promoters of all the selected and clearly annotated top tier genes were separately mapped on the genome-wide alignment of all promoter associated CpG islands. The genes were selected if they were located less than 9 ancestral nodes from an established list of 56 markers (see BROAD analysis). Using this approach, 100 genes were identified.

BROAD analysis: Genome-wide Promoter Alignment

The “Database of Transcription Start Sites” (DBTSS) (Suzuki et al., 2004) mapped each transcript sequence on the human draft genome sequence to identify its transcriptional start site, providing more detailed information on distribution patterns of transcriptional start sites and adjacent regulatory regions. From ˜14,500 well-characterized human genes present in the Affymetrix GeneChip Human Genome U133A Arrays 8793 sequences were extracted from the DBTSS [5, 6] (DBTSS, version 3.0 based on human assembly build 31). The remaining genes (14,500−8793=5707) on the Affymetrix array contained no reported transcriptional start site (TSS) according to DBTSS. All the promoter sequences were subsequently aligned by clustalw algorithm (Li 2003; Thompson et al., 1994) Treeillustrator (Trooskens et al., 2005) was used to visualize the large guide tree in addition to indicating the location of the known markers. Some regions on the “circle” are denser in known markers than others, indicating that there might be a sequence mechanism located in the small region around the TSS which makes certain genes more methylation-prone.

Step 2: As shown by Schuebel et al. and based on the sequencing project from Sjöblom et al. (Sjöblom et al., 2006), promoter CpG island methylation and subsequent gene silencing of genes known to be mutated in cancer is more frequent than the mutations themselves. Therefore the genes identified by Sjöblom et al. were used to identify possible extra targets from the top or next tiers with a known genetic background in either colon or breast cancer. Taking into account all 6 cell lines, 22 extra genes were found to adhere to this category.

Step 3: A final batch of genes was selected based on their appearance in multiple top tiers of the colorectal cell lines from Schuebel et al. and at least one top tier of the lung cancer cell lines. The same approach was used based on multiple breast cancer cell lines, i.e. MDA-MB-231, MDA-MB-468, MCF7 and T-47D. The next tiers of the breast cancer cell lines were also used, since the overlap between multiple top tiers of these breast cancer cell lines and the top tiers of the lung cancer cell lines was minimal compared to the overlap with the colon cancer cell lines. Sixteen genes were selected out of the colon screen and another 17 out of the breast screen.

After removing the duplicates of genes obtained by these different approaches, a list of in total 144 genes was identified by this strategy.

### Verification Strategy

This strategy was applied on a selection of the top and next tier genes of 4 adenocarcinoma cell lines (NCI-H23, NCI-H1568, NCI-H1993, and NCI-H838) and 4 squamous cell carcinoma cell lines (NCI-1520, NCI-H2170, NCI-H1869, and SK-MES-1). These genes were verified in cell lines and/or primary tumors and normal lung samples for expression by reverse transcription-PCR and promoter methylation by MSP. Using this strategy, a list of in total 63 was identified.

Duplicates, imprinted genes and genes for which primer design was not possible were excluded from both lists. This final selection of genes was further analyzed on the Base5 methylation profiling platform (Straub et al. 2007).

### Sample Specimen

A total of 132 samples (64 lung cancer samples, the majority derived from lung adenocarcinoma and sqaumous cell carcinomas; and 68 corresponding normal tissues) were used to find markers which distinguish cancer from non-cancer tissue based on methylation status.

### DNA Extraction and Bisulfite Modification

A high throughput, real-time methylation specific detection platform was applied on two groups of samples totaling 132 genomic DNA samples. The two groups of samples consisted of 64 samples isolated from lung cancer tissue and 68 samples isolated from corresponding normal lung tissue.

From each sample, up to 1 μg of genomic DNA was converted using a bisulfite based protocol (EZ DNA Methylation Kit™, ZYMO Research, Orange, Calif.).

### Detection of Hypermethylation

After conversion and purification the equivalent of 25-75 ng of the starting material was applied per sub-array of an OpenArray™ plate on a real-time qPCR system (BioTrove Inc.) using the DNA double strand-specific dye SYBRgreen for signal detection.

The cycling conditions were: 90° C.-10 seconds, (43° C. 18 seconds, 49° C. 60 seconds, 77° C. 22 seconds, 72° C. 70 seconds, 95° C. 28 seconds) for 40 cycles, 70° C. for 200 seconds, 45° C. for 5 seconds. A melting curve was generated in a temperature range between 45° C. and 94° C. Methylation specific PCR (MSP) primers were designed for each of the genes assessed for hypermethylation.

### Analysis of Methylation

For each combination of assays and samples two parameters were collected using an algorithm which is part of the standard data analysis package offered by the supplier. The parameters were the Ct value (threshold cycle number) of the assessed amplicon and the melting temperature of the assessed amplicon.

The following data analysis workflow was applied to the results created by the software which came with the system OpenArray™ system. Data was collected for each combination of assays and samples in the two sets of samples used. Results were filtered using the following approach. Read outs from not loaded reaction spaces were removed from analysis. Technical Control assays were removed from the data set. Assays known to not work for other than biological reasons were removed from the analysis. Samples for which Ct calls for the gene beta-Actin were not present were removed from the analysis. Ct values >0 for each gene were normalized using the Ct values collected for the gene beta-Actin. This resulted in two files containing the results for each set of sample.

Two robust data analyses designed to cope with inherent variance (i.e., noise) in measured Ct and Tm values were applied which have common features and data analysis steps. Based on the original data, a p-value was assigned to each marker that corresponds to the probability of obtaining Ct/Tm values at least as favorable assuming these values were the result of chance alone. Next, robustness of the above p-value was computed by introducing increasing levels of noise in the data and recomputing the p-value (pVal) as above. The noise level on the x-axis was plotted against (1−pVal) on the y-axis, and the area under the resulting curve was used as the final score for a particular marker. With robust markers, the initial p-value survives for a while, hence (1−pVal) will stay high for a while, hence the area under the curve (AUC) will tend to be high. With not-so-robust markers an initial (1−pVal) will drop quickly with increasing noise levels on the x-axis, which will result in a lower AUC.

The two analysis methods, called “Ranks” and “Squares,” differ only in the way the p-values for each noise level are applied.

### The “Ranks” Method

For computing p-values with the Ranks method for a particular marker, four lists of ranks of samples are generated: two based on the Ct values determined for each assay applied to all samples (cancer samples as well as non cancer samples) resulting in one ascending list of ranks and on one descending list of ranks; and two based on the Tm values determined for each assay applied to all samples (cancer samples as well as non-cancer samples) resulting in one ascending list of ranks and on one descending list of ranks.

For each of these four lists of ranks, the sum of the ranks of the cancer samples are calculated. The lowest of these four sums is kept. Depending on this lowest sum, we label the marker as a positive/negative Ct/Tm marker. For instance, if the lowest sum is found with the descending Ct ranking, we label the marker as a negative Ct ranker; alternatively, in case the lowest sum is found with the descending Tm ranking, the marker is labeled as a positive Tm ranker.

Next, the rank sum of the cancers is recorded for 10,000 random rankings. The fraction of cases where this sum is at least as low as the rank sum of the cancers in the original ranking is taken to be the p-value.

In order to asses the correlation between added noise and resulting p-values, random noise is introduced into the list Ct values and Tm values and the ranking procedure is repeated. This process resulted in a series of p-values with increasing levels of noise which was used to determine an AUC score. Assays are ranked based on their AUC from high to low.

### The “Squares” Method

Applying this method, a lower and/or upper limit is imposed on the Ct and/or Tm values determined for all samples. Such limits correspond to a “square” imposed on the scatter plot of samples where Ct forms the x-axis and Tm forms the y-axis. When considering all possible squares in this scatter plot, we are in fact exploring all combinations of a lower and/or upper limit in the Ct dimension on the one hand and the Tm dimension on the other hand. The sensitivity and specificity for the detection of cancers is determined for the set of all possible squares as defined above.

Next, for each square, the p-value is computed using the Fisher exact test. The square resulting in the highest sensitivity and specificity for determining methylation in cancer and normal samples can thus be determined for each marker candidate.

To test quality of the best square, an increasing amount of noise is injected as described above, and the p-value is recomputed using the Fisher exact test. When plotting the correlation between injected noise and the resulting p-values, the AUC can be determined. The most optimal square will result in the highest AUC. Assays are ranked based on the maximal AUC achievable.

The results of the applied analysis methods are “zipped” together in the following way. The results of applying the two analysis methods described above to two different sample sets are included into four different lists called “sample_set_{—}1_ranks”, “sample_set_{—}2_ranks”, “sample_set_{—}1_squares”, and “sample_set_{—}2_squares”

A new “zipped” list is created by taking the highest scoring assay from the list “sample_set_{—}1_ranks,” followed by a comparison of the highest scoring assay from list “sample_set_{—}2_ranks.” If the marker is already present in the zipped list, this finding is noted and the next highly scoring marker of the list “sample_set_{—}2_ranks” is used. This selection procedure is applied comparing the highest scoring assay of lists “sample_set_{—}1_squares,” noting down if the assay already has scored in the zipped list up to this step. The “sample_set_{—}2_squares” list is used as the source for the next markers in the zipped list. The sequence of lists is maintained until all the assays in all the lists have been assessed.

The cut-offs 0.832, 0.909, 0.687 and 0.743 were applied on the “AUC” determined for each assay and rank in the lists sample_set_{—}1_ranks, sample_set_{—}2_ranks, sample_set_{—}1_squares, and sample_set_{—}2_squares. This resulted in 10 different genes.

Results

A high throughput, real-time methylation specific detection platform was applied on two groups of samples isolated from lung cancer tissue and from corresponding normal lung tissue. In this study it was shown that a number of genes are differentially methylated in lung cancer, in particular in non-small cell lung cancer, more particularly in lung adenocarcinoma or squamous cell carcinoma. We identified 64 different assays for detecting 49 different genes being differentially methylated in human lung cancer tissue and normal lung tissue control samples. The genes identified are ACSL6, ALS2CL, APC2, BEX1, BMP7, CBR3, CD248, CD44, CHD5, DLK1, DPYSL4, DSC2, EPB41L3, EPHB6, ERBB3, FBLN2, FBN2, FOXL2, GSTP1, HS3ST2, IGFBP7, IRF7, JAM3, LOX, LY6D, LY6K, MACF1, MCAM, NEFH, NID2, PCDHB15, PHACTR3, POMC, PRKCA, PSEN1, RBP1, RRAD, SFRP1, SOD3, SOX17, SULF2, TIMP3, TJP2, TRPV2, UCHL1, WDR69, ZFP42, ZNF442, ZNF655.

The resulting assays have the assay details provided in Table 1.

TABLE 1
Methylation Specific PCR (MSP) primers used for the 64 assays:

| | | | | Sense | Antisense |
|---|---|---|---|---|---|
| | | Entrez | Official Gene | primer sequence | primer sequence |
| # | Assay Name | GeneID | symbol | (SEQ ID NO: 1-64, respectively) | (SEQ ID NO: 65-128, respectively) |
| 1 | ACSL6_17822 | 23305 | ACSL6 | TTTAATGTTACGTTTTGGCGTT | GAACCAACCCTCTCCGACC |
| 2 | ACSL6_17824 | 23305 | ACSL6 | GCGGTTGTAAGGTTTTTGGTC | ATTTTTCCGCAACCTCTCG |
| 3 | ALS2CL_bay | 259173 | ALS2CL | GGACGGGTGTTTGCGTTTTAC | CGAAACCAAAAAACTAAACGAAAACCG |
| 4 | APC2 | 10297 | APC2 | GTCGTTTGTTTAGGTTCGGATC | GACCCGAAATAACCTCGAAACG |
| 5 | BEX1_12842 | 55859 | BEX1 | TCGGGGTTTTTATTTGGTTC | AATCGTCACTCGTATCTCGCT |
| 6 | BMP7_17905 | 655 | BMP7 | GTACGTGCGTTTATTGCGAG | CGTTATCCAAACTAAAATCGACC |
| 7 | CBR3_17931 | 874 | CBR3 | GGTATCGGTTTGGTTATCGC | CGCCTACAACTACTACACGACC |
| 8 | CBR3_17935 | 874 | CBR3 | GTTTTCGATTGATTTATTAAGGTTC | TCAAAATCCGAACTCTAAACCG |
| 9 | CD248_17939 | 57124 | CD248 | TCGTGGGAAGAGAGCGTAG | TTACTAACCTAAACGACCGCAA |
| 10 | CD248_17946 | 57124 | CD248 | TTTTGTTAAGAGTTGTCGTTAGTTC | AATATAAACCCTACGACCGCC |
| 11 | CD248_17947 | 57124 | CD248 | GGGGTAGTCGTTAATTGCGT | TCTTCCCCGAAAACCGCTA |
| 12 | CD44_17961 | 960 | CD44 | CGGGAGAAGAAAGTTAGTGCGT | AAATCGAAAAACCTAAAATATCGC |
| 13 | CHD5_bay | 26038 | CHD5 | GAGCGTTCGGGTTTTGC | CGACCTCGACGAAAAAATAACG |
| 14 | CRBP_1 | 5947 | RBP1 | TTGGGAATTTAGTTGTCGTCGTTTC | AAACAACGACTACCGATACTACGCG |
| 15 | DLK1_18031 | 8788 | DLK1 | GAGGTTTGCGGTTTAGGTTC | CTCACACTATACAACACGCGAC |
| 16 | DLK1_18033 | 8788 | DLK1 | GGAGTTGGGGTTTACGAGAC | ATAATAAATTCCCCGACGACC |
| 17 | DPYSL4_18047 | 10570 | DPYSL4 | GGTGTTTTGATAGAAGTCGTTAGTC | AAAACCATTAACGCCCACG |
| 18 | DPYSL4_18050 | 10570 | DPYSL4 | GGGGTTATAGTTTGGCGTTC | GCTCTAAAAACCACACCCGTC |
| 19 | DSC2_18056 | 1824 | DSC2 | GGTTTCGGTTTCGTTTTGTTC | CTCTACGACTCAAACCTCGCT |
| 20 | EPB41L3_19071 | 23136 | EPB41L3 | GGGATAGTGGGGTTGACGC | ATAAAAATCCCGACGAACGA |
| 21 | EPB41L3_19072 | 23136 | EPB41L3 | GCGTGGGTTTTCGTCGTAG | CCCAAAACTACTCGCCGCT |
| 22 | EPHB6_bay | 2051 | EPHB6 | GGGTGTTCGATTTAAGTCGAGTTC | CGCGAATCTTAACCGAAAAAATCG |
| 23 | ERBB3_18097 | 2065 | ERBB3 | GTTTAGTTAAGTTCGGTTCGGG | GATTACAATTTACAACCTCCGCT |
| 24 | ERBB3_18099 | 2065 | ERBB3 | AGGGAGTTTAGTTAAGTTCGGTTC | TACAACCTCCGCTACCGTC |
| 25 | FBLN2_13328 | 2199 | FBLN2 | TAGAGCGGAGGAAGTTGCG | CAAATACGAACACAAAAACCGA |
| 26 | FBN2_18150 | 2201 | FBN2 | TCGGAGTTTTATAGGGTAACGAA | CTCTTACTAACCGCACGCC |
| 27 | FBN2_18151 | 2201 | FBN2 | TTGGAGATTTCGATAGAGCGT | AAACTACCGACTACACCTCCG |
| 28 | FOX-L2 | 668 | FOXL2 | GCGATAGGTTTTTAGTAAGTAAGCGC | CTCTCCGCTCCAAACGCTAACGCG |
| 29 | Gst-Pi | 2950 | GSTP1 | TTCGGGGTGTAGCGGTCGTC | GCCCCAATACTAAATCACGACG |
| 30 | HS3ST2_19130 | 9956 | HS3ST2 | ACGTAAGAGTTTGGGAGCGT | GACTCCTCGAAAAACAAACGA |
| 31 | HS3ST2_19131 | 9956 | HS3ST2 | GTTTCGGGGTTCGTTTTTC | CGACTCGCTCTATCTCGCAC |
| 32 | IGEBP7_19196 | 3490 | IGFBP7 | TTTGTCGGCGTCGTTATTTTC | AAACTACCTACTAAACGAAACCCG |
| 33 | IGFBP7_19200 | 3490 | IGFBP7 | CGTTTATGGGTCGGTTACGTC | ATAAAAACACGAAAACCCCGC |
| 34 | IRF7_18346 | 3665 | IRF7 | AGTTGAGAATCGGACGGGG | AACGAATCAAACTCCCGAAA |
| 35 | JAM3 | 83700 | JAM3 | GGGATTATAAGTCGCGTCGC | CGAACGCAAAACCGAAATCG |
| 36 | LOX_18967 | 4015 | LOX | GCGCGTAGAGTTGTAAAGGTTC | ACGTCCTCCTCGAACGAAA |
| 37 | LOX_18977 | 4015 | LOX | GGTAGAGGCGAGGAGTTGTTC | TACACAAACCGTTCTAACCCGA |
| 38 | LY6D_8402 | 8581 | LY6D | GATGTCGTTTGGGAGTAGTGC | ACAAAATACCGCTAACTAACGAA |
| 39 | LY6K | 54742 | LY6K | GCGGGGTTTTTTTTATCGGTTAGATTC | CAACGATACCCAAAAAAAATCACGCG |
| 40 | MACF1_bay | 23499 | MACF1 | GTTTTCGTTGTCGTTACGGGTTC | GCGCAACGAACAAAACG |
| 41 | MCAM | 4162 | MCAM | AGAATTTAGGTCGGTTTTTATCG | ACGCAAAATTCTTCTCCCAAAA |
| 42 | NEFH_18452 | 4744 | NEFH | GTCGGATGAAGTATTCGGG | CCCTACAAACGACGACGAAC |
| 43 | NID2_9093 | 22795 | NID2 | TTATTTCGTTTTTAGGGAGTTTTC | CTTACGAACCATTTAATCCCG |
| 44 | NID2_9094 | 22795 | NID2 | TTTCGTGTGGGAAGAGTTCGT | CGAATAACCGAACGACCGATA |
| 45 | PCDHB15_10763 | 56121 | PCDHB15 | TTTTGGTTATTAGGTAGTTCGGTTC | CACTCTTCGTACTATTCCCGCT |
| 46 | PHACTR3_11692 | 116154 | PHACTR3 | TTATTTTGCGAGCGGTTTC | GAATACTCTAATTCCACGCGACT |
| 47 | POMC | 5443 | POMC | GATTTGGGCGTTTTTGGTTTTTCGC | GACTTCTCATACCGCAATCG |
| 48 | PRKCA_18626 | 5578 | PRKCA | GGGCGTTGAGGTAGAAGAAC | CGACACCTACCAAATAAAATCG |
| 49 | PSEN1_18648 | 5663 | PSEN1 | TTAGGTCGGAGGTTTCGTTT | AAACCCTCACCGTTATCGTC |
| 50 | RRAD_18698 | 6236 | RRAD | GATGTTTCGGTCGAGGTTTC | AAACGACTACAAATAAATACGCCA |
| 51 | SFRP1 | 6422 | SFRP1 | TGTAGTTTTCGGAGTTAGTGTCGCGC | CCTACGATCGAAAACGACGCGAACG |
| 52 | SFRP1_9381 | 6422 | SFRP1 | TTTTGTTCGTCGTATTTTCGG | ATAACGACCCTCGACCTACGAT |
| 53 | SOD3_18740 | 6649 | SOD3 | AGTATAGAGTGGGGAGCGTAGC | CTTTCCTACCACCGAAACGA |
| 54 | SOX17 | 64321 | SOX17 | TTGCGTTAGTCGTTTGCGTTC | CAAAAACGAATCCCGTATCCGACG |
| 55 | SULF2_bay | 55959 | SULF2 | GTTAGTCGAGTTCGGAGGTATC | CAACTCCGAACGAAACAATAAACG |
| 56 | TIMP3 | 7078 | TIMP3 | GCGTCGGAGGTTAAGGTTGTT | CTCTCCAAAATTACCGTACGCG |
| 57 | TJP2_18792 | 9414 | TJP2 | CGGGTTAGAGTATTGTTCGGT | GAACACAAATCCCGCGTAA |
| 58 | TJP2_18797 | 9414 | TJP2 | GATTTTATCGGGGAAATATCG | AAACAAATCCCGCTCCGAA |
| 59 | TRPV2_18803 | 51393 | TRPV2 | TTATTTCGTAGGTTGAGGTTAGGGC | TCCTCTACTATCAACGCCGAC |
| 60 | UCHL1 | 7345 | UCHL1 | GTTGTATTTTCGCGGAGCGTTC | CTCACAATACGTCTAACCGACG |
| 61 | WDR69_18844 | 164781 | WDR69 | GTTTAGGTTGTGGTTTAGGTCGTC | ACACCTCGTATCCTCACTAAAAACG |
| 62 | ZFP42_bay | 132625 | ZFP42 | GGGGTTTTTAGGTATTCGGTTCGTAC | AATACGCAATACCCGACGACCG |
| 63 | ZNF442_bay | 79973 | ZNF442 | TCGGTTTTTAGTTTTTTCGGTCGC | CAATTACTACGCAAAAACGAAACAAAACG |
| 64 | ZNF655 | 79027 | ZNF655 | TTATCGAGAAGCGTCGGTTTC | ACCGAAAAAAAAAACGAACCTAACCG |

TABLE 2
Amplicon details
Amplicon details (converted sequence):

| | | | Official | |
|---|---|---|---|---|
| | | Entrez | Gene | |
| | Assay Name | GeneID | symbol | Amplicon Sequence (converted) (SEQ ID NO: 129-192, respectively) |
| 1 | ACSL6_17822 | 23305 | ACSL6 | TTTAATGTTACGTTTTGGCGTTCGTCGTTCGTGTTTTTTTTTTTAGTCGGTTTTCGTAGAATGTTAGG |
| | | | | TATTGACGTTGGAGAGCGGGGTCGGAGAGGGTTGGTTC |
| 2 | ACSL6_17824 | 23305 | ACSL6 | GCGGTTGTAAGGTTTTTGGTCGGTGAGTGAATTAGTAGGTAAGGATGGTAGTTAGGGTATTTATATTT |
| | | | | ACGAGGGTGGTGGTCGAGAGGTTGCGGAAAAAT |
| 3 | ALS2CL_bay | 259173 | ALS2CL | GGACGGGTGTTTGCGTTTTACGTTTAGTTCGTTTAGGIGGGGGTTTTCGTTTTTTCGGTTGTTGCGGT |
| | | | | TTTCGTTTAGTTTTTTGGTTTCG |
| 4 | APC2 | 10297 | APC2 | GTCGTTTGTTTAGGTTCGGATCGGGTTTTGTICGTITCGGAGTTTTTGTTCGCGTCGCGGAGATTTCG |
| | | | | GAGTTCGCGCGTTTCGAGGTTATTTCGGGTC |
| 5 | BEX1_12842 | 55859 | BEX1 | TCGGGGTTTTTATTTGGTTCGTTTTTTTTCGGGTCGGATGTTAGTTCGTCGAGCGTAGGGTAGCGGGG |
| | | | | AGTTGGTAGCGAGATACGAGTGACGATT |
| 6 | BMP7_17905 | 655 | BMP7 | GTACGTGCGTTTATTGCGAGTTGCGGCGTCGTATAGTTTCGTGGCGTTTTGGGTATTTTTGTTTTTGT |
| | | | | TGCGTTTCGTTTTGGTCGATTTTAGTTTGGATAACG |
| 7 | CBR3_17931 | 874 | CBR3 | GGTATCGGTTTGGTTATCGCGCGCGAATTGTGTCGATAGTTTTTTGGGGATGTGGTGTTTATCGCGCG |
| | | | | GGACGTGGCGCGGGGITAGGCGGTCGTGTAGTAGTTGTAGGCG |
| 8 | CBR3_17935 | 874 | CBR3 | GTTTTCGATTGATTTATTAAGGTTCGATTTGGTTTCGGATATTTCGTAGATTATTTCGCGGTTTAGAG |
| | | | | TTCGGATTTTGA |
| 9 | CD248_17939 | 57124 | CD248 | TCGTGGGAAGAGAGCGTAGTAGTTGTTGGGGTCGTAGGCGGTACGGGGTTTAGTAGTTTAGGGGTTTT |
| | | | | GGTTTAGTGTGGGTTTTGCGGTCGTTTAGGTTAGTAA |
| 10 | CD248_17946 | 57124 | CD248 | TTTTGTTAAGAGTTGTCGTTAGTTCGGGGTCGGATTAGTTCGGGGGTATCGCGATGTTGTTGCGTTTG |
| | | | | TTGTTGGTTTGGGCGGTCGTAGGGTTTATATT |
| 11 | CD248_17947 | 57124 | CD248 | GGGGTAGTCGTTAATTGCGTTTTTTTTTTTTTTTCGTTTTTAATTTTAGAGTTTTTTATTTTATTGTT |
| | | | | TTTTGTTTTAGCGGTTTTCGGGGAAGA |
| 12 | CD44_17961 | 960 | CD44 | CGGGAGAAGAAAGTTAGTGCGTTTTTGGGCGTAGGGGTTAGTGGGGTTCGGAGGTATAGGTATTTCGC |
| | | | | GATATTTTAGGTTTTTCGATTT |
| 13 | CHD5_bay | 26038 | CHD5 | GAGCGTTCGGGTTTTGCGGGGAGTAGGTTAAGGCGGTCGAGAGAAAGGGGGGTCGAGACGGGGGGGTG |
| | | | | GAGGTTTGGGGGGGTGGGGGGGTAGGCGGTCGTTATTTTTTCGTCGAGGTCG |
| 14 | CRBP_1 | 5947 | RBP1 | TTGGGAATTTAGTTGTCGTCGTTTCGTAGAGTTTTTTGTTTTCGGAGGGCGTTTATTTTCGGGTCGTT |
| | | | | TATTATTCGCGTAGTATCGGTAGTCGTTGTTT |
| 15 | DLK1_18031 | 8788 | DLK1 | GAGGTTTGCGGTTTAGGTTCGATTTTTGCGATTTGTTTTAGGTAGGTTTGTATGTGCGCGGCGGTCGC |
| | | | | GTGTTGTATAGTGTGAG |
| 16 | DLK1_18033 | 8788 | DLK1 | GGAGTTGGGGTTTACGAGACGGGGCGTGCGGGGTATCGGGCGGTCGGCGGGGAGTCGTAGGTTTTTTT |
| | | | | AGAGGGGGCGCGAGTCGGGTCGTCGGGGAATTTATTAT |
| 17 | DPYSL4_18047 | 10570 | DPYSL4 | GGTGTTTTGATAGAAGTCGTTAGTCGGTGTTATGTTTAGGATAGGTATTTGTAGTTTTGTGTGGACGT |
| | | | | GTAACTTATTAGGAAGGATTATTAGGTCGTGGGCGTTAATGGTTTT |
| 18 | DPYSL4_18050 | 10570 | DPYSL4 | GGGGTTATAGTTTGGCGTTCGGATTTTGGTTCGGGTTATTTGCGAAGGAGTCGGTTTTGGTTAAGGTG |
| | | | | TTTTTTTGGACGGGTGTGGTTTTTAGAGC |
| 19 | DSC2_18056 | 1824 | DSC2 | GGTTTCGGTTTCGTTTTGTTCGTTGTTTTCGGCGACGGTCGTGGTTTTTGTTTTGGGGTTAATTATAG |
| | | | | AGCGAGGTTTGAGTCGTAGAG |
| 20 | EPB41L3_19071 | 23136 | EPB41L3 | GGGATAGTGGGGTTGACGCGTGGTTTCGGCGTCGCGCGGTTTTTCGAATTTCGAGTTTCGCGTTCGGC |
| | | | | GCGGTCGGGGTTTTTAATCGTTTTTTCGTTCGTCGGGATTTTTAT |
| 21 | EPB41L3_19072 | 23136 | EPB41L3 | GCGTGGGTTTTCGTCGTAGTTTCGCGGAGTTTCGGTGTTTTTTGTAATAGGGGGCGGGGGGAATAGCG |
| | | | | CGGAGTAGTTTTGGG |
| 22 | EPHB6_bay | 2051 | EPHB6 | GGGTGTTCGATTTAAGTCGAGTTCGAGTTCGAGTTTAGGTAGGAGTTTTATAGATAGTTTTTTTTTTT |
| | | | | TTTTATTTTTTGTAGGCGTTTTACGCGTGCGATTTTTCGGTTAAGATTCGCG |
| 23 | ERBB3_18097 | 2065 | ERBB3 | GTTTAGTTAAGTTCGGTTCGGGGGTTTTTAGGTTAGGATATCGAGGTAAGAGTTATTTGAATCGTTGG |
| | | | | ACGATTGGTGGTTGTTGCGGCGACGGTAGCGGAGGTTGTAAATTGTAATC |
| 24 | ERBB3_18099 | 2065 | ERBB3 | AGGGAGTTTAGTTAAGTTCGGTTCGGGGGTTTTTAGGTTAGGATATCGAGGTAAGAGTTATTTGAATC |
| | | | | GTTGGCGAATTGGTGGTTGTTGCGGCGACGGTAGCGGAGGTTGTA |
| 25 | FBLN2_13328 | 2199 | FBLN2 | TAGAGCGGAGGAAGTTGCGGATfTGGGGTGGGGGAATTCGTTCGCGGATTTTTGGTTTTTATTTCGCG |
| | | | | GTCGTTTTTGTGTTCGTATTTG |
| 26 | FBN2_18150 | 2201 | FBN2 | TCGGAGTTTTATAGGGTAACGAAGCGCGGGTAGCGGTTGCGGAGTCGGGCGGAGGTGCGCGGGGTCGG |
| | | | | GGCGTGCGGTTAGTAAGAG |
| 27 | FBN2_18151 | 2201 | FBN2 | TTGGAGATTTCGATAGAGCGTCGGTTTTTTGATTGTTCGCGAAGCGAGACGCGGGGCGTCGGGTTTAG |
| | | | | CGTAGTGAGCGGCGAGGCGCGGCGGAGGTGTAGTCGGTAGTTT |
| 28 | FOX-L2 | 668 | FOXL2 | GCGATAGGTTTTTAGTAAGTAAGCGCGGGCGGTATTCGTAGTTTTTAGAAGTTTGAGATTTGGTCGTA |
| | | | | AGCGGATTCGTGCGTTTTAATTTTTTGTCGCGTTAGCGTTTGGAGCGGAGAG |
| 29 | Gst-Pi | 2950 | GSTP1 | TTCGGGGTGTAGCGGTCGTCGGGGTTGGGGTCGGCGGGAGTTCGCGGGATTTTTTAGAAGAGCGGTCG |
| | | | | GCGTCGTGATTTAGTATTGGGGC |
| 30 | HS3ST2_19130 | 9956 | HS3ST2 | ACGTAAGAGTTTGGGAGCGTTCGAGTCGTTCGGTTGTTCGGAGTTTTATCGTTTAGGATCGGGAGATGT |
| | | | | TGGAAATGTAATCGTTTGTTTTTCGAGGAGTC |
| 31 | HS3ST2_19131 | 9956 | HS3ST2 | GTTTCGGGGTTCGTTTTTCGGTAGGTTCGGGGAGAGGTGGGGTGATAATGGGTTGGGGTGCGCGCGTGT |
| | | | | TTTATAGGTGCGAGATAGAGCGAGTCG |
| 32 | IGFBP7_19196 | 3490 | IGFBP7 | TTTGTCGGCGTCGTTATTTTCGTACGGTTCGTTTTCGTCGCGGGCGTATATAGGGTAGTAGTCGTACGC |
| | | | | GTCGCGGGTTTCGTTTAGTAGGTAGTTT |
| 33 | IGFBP7_19200 | 3490 | IGFBP7 | CGTTTATGGGTCGGTTACGTCGGGTGTTCGTTTATTTTTCGACGTTAGTAGGAGCGCGCGCGTAGGTTT |
| | | | | CGCGGGGTCGGGAGGGCGGTACGGGCGGGGTTTTCGTGTTTTTAT |
| 34 | IRF7_18346 | 3665 | IRF7 | AGTTGAGAATCGGACGGGGTGGGATCGAGGAGGGTGCGAAGCGTTATTGTTTAGGTTTCGTTTTTTCGG |
| | | | | GAGTTTGATTCGTT |
| 35 | JAM3 | 83700 | JAM3 | GGGATTATAAGTCGCGTCGCGTTGTCGTTGGTTTTTTAGTAATTTTCGATATGGCGTTGAGGCGGTTAT |
| | | | | CGCGATTTCGGTTTTGCGTTCG |
| 36 | LOX_18967 | 4015 | LOX | GCGCGTAGAGTTGTAAAGGTTCGAGTAGGAGTACGGTTTAGGCGAAGCGTATTATTTTTTTTGTTAGAT |
| | | | | TGATTTCGTTCGAGGAGGACGT |
| 37 | LOX_18977 | 4015 | LOX | GGTAGAGGCGAGGAGTTGTTCGTTTTGTACGTTTTTAATCGTATTACGTGAATAAATAGTTGAGGGGCG |
| | | | | GTCGGGTTAGAACGGTTTGTGTA |
| 38 | LY6D_18402 | 8581 | LY6D | GATGTCGTTTGGGAGTAGTGCGGGTTTTTGTATTGTTAAGGTTTTATAGGTACGGGTTGGGCGGGGGTG |
| | | | | GGTAGTTCGTTAGTTAGCGGTATTTTGT |
| 39 | LY6K | 54742 | LY6K | GCGGGGTTTTTTTTATCGGTTAGATTCGGGGAGAGGCGCGCGGAGGTTGCGAAGGTTTTAGAAGGGCGG |
| | | | | GGAGGGGGCGTCGCGCGTTGATTTTTTTTGGGTATCGTTG |
| 40 | MACF1_bay | 23499 | MACF1 | GTTTTCGTTGTCGTTACGGGTTCGTTTTTTTTTTTTTTCGGTTTTTAGGGTAAGGCGCGGGGCGCGGGG |
| | | | | TTGGATGTAGGCGTTTTGTTCGTTGCGC |
| 41 | MCAM | 4162 | MCAM | AGAATTTAGGTCGGTTTTTATCGTTTTTTAGAACGATTGTATTATTGTCGTTGTCGTCGGTTTGATATT |
| | | | | GTTTTAGTTTTAGTGTTGGTAGTTTTGGGAGAAGAATTTTGCGT |
| 42 | NEFH_18452 | 4744 | NEFH | GTCGGATGAAGTATTCGGGCGTTTTTATTGCGGAAGGGCGGGGATGGTTGTGACGTAGGCGTGTTCGTC |
| | | | | GTCGTTTGTAGGG |
| 43 | NID2_9093 | 22795 | NID2 | TTATTTCGTTTTTAGGGAGTTTTCGGGTTATTTTTTTATTCGGGTTGTTTCGCGGTTTTTAAGGAGTTT |
| | | | | TATTTTCGGGATTAAATGGTTCGTAAG |
| 44 | NID2_9094 | 22795 | NID2 | TTTCGTGTGGGAAGAGTTCGTTTGGGTGTAGCGTCGCGGTTCGTAATATTAGTAAGGGTAGTAGTAGTA |
| | | | | GTATTGGTAACGACGATAGTATCGGTCGTTCGGTTATTCG |
| 45 | PCDHB15_10763 | 56121 | PCDHB15 | TTTTGGTTATTAGGTAGTTCGGTTCGGCGGTTCGTTCGGGGTATTAGTTCGGTGTAGGGCGCGGAGTCG |
| | | | | TTTTGTAGCGGGAATAGTACGAAGAGTG |
| 46 | PHACTR3_11692 | 116154 | PHACTR3 | TTATTTTGCGAGCGGTTTCGCGATACGAGGTAGTCGTTTTCGTTTTTCGACGCGGTTATGGGTTCGGTC |
| | | | | GGCGCGGGGGTAAGTTAGAGCGAGTCGCGTGGAATTAGAGTATTC |
| 47 | POMC | 5443 | POMC | GATTTTGGGCGTTTTTGGTTTTTCGCGGTTTCGAGTTTTCGATAAATTTTTGCGTCGATTGCGGTATG |
| | | | | AGAAGTC |
| 48 | PRKCA_18626 | 5578 | PRKCA | GGGCGTTGAGGTAGAAGAACGTGTACGAGGTGAAGGATTATAAATTTATCGCGCGTTTTTTTAAGTAG |
| | | | | TTTATTTTTTGTAGTTATTGTATCGATTTTATTTGGTAGGTGTCG |
| 49 | PSEN1_18648 | 5663 | PSEN1 | TTAGGTCGGAGGTTTCGTTTTTTTTTTTTTGGTTTTTTTTTTTTTTCGTGGGTCGGTCGTTAACGACG |
| | | | | TTAGAGTCGGAAATGACGATAACGGTGAGGGTTT |
| 50 | RRAD_18698 | 6236 | RRAD | GATGTTTCGGTCGAGGTTTCGTCGTAGTTTTTTTTTAGTTTTTAGGTCGCGGCGTTTTTATTCGGGAT |
| | | | | TTTTTCGGATTTGGCGTATTTATTTGTAGTCGTTT |
| 51 | SFRP1 | 6422 | SFRP1 | TGTAGTTTTCGGAGTTAGTgtcgcgcgttcgtcgtttcgcgttTTTTTGTTCGTCGTATTTTCGGGAG |
| | | | | TCGGGGCGTATTTAGTTCGTAGCGTCGTTTTTTCGTTCGCGTCGTTTTCGATCGTAGG |
| 52 | SFRP1_9381 | 6422 | SFRP1 | TTTTGTTCGTCGTATTTTCGGGAGTCGGGGCGTATTTAGTTCGTAGCGTCGTTTTTTCGTTCGCGTCG |
| | | | | TTTTCGATCGTAGGTCGAGGGTCGTTAT |
| 53 | SOD3_18740 | 6649 | SOD3 | AGTATAGAGTGGGGAGCGTAGCGACGAAGAATGAATAGGGTTTCGTGAGGTTTTAAATATTCGTTTCG |
| | | | | GTGGTAGGAAAG |
| 54 | SOX17 | 64321 | SOX17 | TTGCGTTAGTCGTTTGCGTTCGTTTTTAGTTTATATTATGAAAGCGTTTATCGGTCGTCGGATACGGG |
| | | | | ATTCGTTTTTG |
| 55 | SULF2_bay | 55959 | SULF2 | GTTAGTCGAGTTCGGAGGTATCGGGAGGTCGAGAGTCGTCGGGATTTTAGTTTTGCGTTTATTGTTTC |
| | | | | GTTCGGAGTTG |
| 56 | TIMP3 | 7078 | TIMP3 | GCGTCGGAGGTTAAGGTTGTTTCGTACGGTTCGGCGGGCGAGCGAGTTCGGGTTGTAGTAGTTTCGTCG |
| | | | | GCGGCGCGTACGGTAATTTTGGAGAG |
| 57 | TJP2_18792 | 9414 | TJP2 | CGGGTTAGAGTATTGTTCGGTGGTGTTTAGGAGGAGTAGGAGTAGGAGTAGAAGTAGAAGCGGGGTTCG |
| | | | | GAGTTGCGCGTTTACGCGGGATTTGTGTTC |
| 58 | TJP2—18797 | 9414 | TJP2 | GATTTTATCGGGGAAATATCGCGGATAGTCGGGTTAGTAGCGTTCGGAGTTTATTTTAGGTTTTTAAAT |
| | | | | TTGTAGTATTTTTTAGAGCGCGCGCGTTCGGAGCGGGATTTGTTT |
| 59 | TRPV2_18803 | 51393 | TRPV2 | TTATTTCGTAGGTTGAGGTTAGGGCGTGGCGGTTGTTGGGATTTCGGAGTTTTTTAGTAGTAGGGGTTG |
| | | | | CGGGAGGAAGTGAAGTCGGGAGGGGTTGTCGGCGTTGATAGTAGAGGA |
| 60 | UCHL1 | 7345 | UCHL1 | GTTGTATTTTCGCGGAGCGTTCGGTAGAAATAGTTTAGGGAAGACGAAAAATAGTTAGCGGAGTCGTTT |
| | | | | AGGTTGTAGTTATAAAGCGTCGGTTAGACGTATTGTGAG |
| 61 | WDR69_18844 | 164781 | WDR69 | GTTTAGGTTGTGGTTTAGGTCGTCGGTTTTCGGTTATGTTTAGTTTTTTTGAGGTCGTTTTTAGTGAGG |
| | | | | ATACGAGGTGT |
| 62 | ZFP42_bay | 132625 | ZFP42 | GGGGTTTTTAGGTATTCGGTTCGTACGTAAATTTTTAGTTCGGGGTTTTTTGATTTTCGCGTTTATTTT |
| | | | | TTTAGTCGGTCGTCGGGTATTGCGTATT |
| 63 | ZNF442_bay | 79973 | ZNF442 | TCGGTTTTTAGTTTTTTCGGTCGCGGGGTGGGAGTTGGGGGTTGGGTCGGTAGTCGGGATTTCGGGCGT |
| | | | | TTTGTTTCGTTTTTGCGTAGTAATTG |
| 64 | ZNF655 | 79027 | ZNF655 | TTATCGAGAAGCGTCGGTTTCGGGGTTGTTTATAGCGGTTCGGGAGAGGTTGTGGTGGTTTCGAGCGCG |
| | | | | AGTGTGTAGGTGATAGGATAGCGGTTAGGTTCGTTTTTTTTTTCGGT |

Amplicon details (non-converted sequence):

| | | | Official | |
|---|---|---|---|---|
| | Assay | Entrez | Gene | |
| | Name | GeneID | symbol | amplicon sequence (not converted) (SEQ ID NO: 193-256, respectively) |
| 1 | ACSL6_17822 | 23305 | ACSL6 | CTCAATGTCACGCTCTGGCGCTCGTCGCCCGTGCTCCCCCTTCCAGCCGGTTTCCGCAGAATGCCAGGT |
| | | | | ACTGACGTTGGAGAGCGGGGCCGGAGAGGGCTGGTTC |
| 2 | ACSL6_17824 | 23305 | ACSL6 | GCGGCTGCAAGGCCTTTGGCCGGTGAGTGAACCAGTAGGCAAGGATGGCAGCCAGGGCACCCATACTCA |
| | | | | CGAGGGTGGTGGCCGAGAGGCTGCGGAAAAAC |
| 3 | ALS2CL_bay | 259173 | ALS2CL | GGACGGGTGTCTGCGCTCCACGCTTAGCTCGTCCAGGTGGGGGCTCCCGCCTCCTCGGCTGCTGCGGT |
| | | | | CCCCGCCCAGCTCCTTGGTCCCG |
| 4 | APC2 | 10297 | APC2 | GCCGCCTGCCCAGGCCCGGACCGGGCTTTGTCCGCCCCGGAGCCCCTGCCCGCGCCGCGGAGACCCC |
| | | | | GGAGCCCGCGCGCTCCGAGGCCACCCCGGGCC |
| 5 | BEX1_12842 | 55859 | BEX1 | CCGGGGCCCTTACCTGGTCCGCTTTCCCCCGGGCCGGATGCCAGCCCGCCGAGCGCAGGGCAGCGGG |
| | | | | GAGCTGGTAGCGAGACACGAGTGACGACT |
| 6 | BMP7_17905 | 655 | BMP7 | GCACGTGCGCTCACTGCGAGCTGCGGCGCCGCACAGCTTCGTGGCGCTCTGGGCACCCCTGTTCCTGCT |
| | | | | GCGCTCCGCCCTGGCCGACTTCAGCCTGGACAACG |
| 7 | CBR3_17931 | 874 | CBR3 | GGCATCGGCTTGGCCATCGCGCGCGAACTGTGCCGACAGTTCTCTGGGGATGTGGTGCTCACCGCGCG |
| | | | | GGACGTGGCGCGGGGCCAGGCGGCCGTGCAGCAGCTGCAGGCG |
| 8 | CBR3_17935 | 874 | CBR3 | GCCCCCGACTGACCCATCAAGGTCCGATTTGGCTTCGGACACCTCGCAGATCACCCCGCGGCTCAGAGC |
| | | | | CCGGATCCTGA |
| 9 | CD248_17939 | 57124 | CD248 | CCGTGGGAAGAGAGCGTAGCAGCTGCTGGGGCCGCAGGCGGCACGGGGCTCAGCAGCCCAGGGGTCC |
| | | | | TGGCCCAGTGTGGGCCCTGCGGCCGCCCAGGCCAGCAA |
| 10 | CD248_17946 | 57124 | CD248 | CCCTGTCAAGAGCTGCCGCCAGCCCGGGGCCGGACCAGTCCGGGGGCATCGCGATGCTGCTGCGCCTG |
| | | | | TTGCTGGCCTGGGCGGCCGCAGGGCCCACACT |
| 11 | CD248_17947 | 57124 | CD248 | GGGGCAGCCGTCAACTGCGCCTTCTCCCCTCCTCCGCCCCCAACCTTAGAGCCCCCCACCCCACTGCTT |
| | | | | CCTGCTCTAGCGGCCCCCGGGGAAGA |
| 12 | CD44_17961 | 960 | CD44 | CGGGAGAAGAAAGCCAGTGCGTCTCTGGGCGCAGGGGCCAGTGGGGCTCGGAGGCACAGGCACCCCG |
| | | | | CGACACTCCAGGTTCCCCGACCC |
| 13 | CHD5_bay | 26038 | CHD5 | GAGCGCCCGGGCTTTGCGGGGAGCAGGCTAAGGCGGCCGAGAGAAAGGGGGGTCGAGACGGGGGGGT |
| | | | | GGAGGTTTGGGGGGGTGGGGGGGCAGGCGGCCGCCATCTTCTCGCCGAGGCCG |
| 14 | CRBP_1 | 5947 | RBP1 | CTGGGAATCCAGCTGTCGCCGCCCCGCAGAGCCCCCTGTCCCCGGAGGGCGCTCATTTCCGGGCCGCC |
| | | | | CACCACCCGCGTAGCACCGGCAGCCGCTGTCC |
| 15 | DLK1_18031 | 8788 | DLK1 | GAGGTCTGCGGCCCAGGTTCGATTCCTGCGACTTGTCCTAGGCAGGCCTGTATGTGCGCGGCGGCCGC |
| | | | | GTGCTGTACAGTGTGAG |
| 16 | DLK1_18033 | 8788 | DLK1 | GGAGTTGGGGCTCACGAGACGGGGCGTGCGGGGCACCGGGCGGCCGGCGGGGAGTCGCAGGCTTCCC |
| | | | | CAGAGGGGGCGCGAGCCGGGCCGCCGGGGAACTCACCAT |
| 17 | DPYSL4_18047 | 10570 | DPYSL4 | GGTGCCCTGACAGAAGTCGTCAGCCGGTGTCATGCCCAGGACAGGCATCTGCAGCCTTGTGTGGACGTC |
| | | | | AACGCCACCAGGAAGGACCATCAGGCCGTGGGCGTCAATGGTCTT |
| 18 | DPYSL4_18050 | 10570 | DPYSL4 | GGGGTCACAGCCTGGCGCTCGGACCCTGGCCCGGGTCATCTGCGAAGGAGCCGGCTTTGGCCAAGGTG |
| | | | | CCTTCCTGGACGGGTGTGGTTCCCAGAGC |
| 19 | DSC2_18056 | 1824 | DSC2 | GGCCCCGGCTCCGCCCTGCCCGCTGCCCTCGGCGACGGCCGTGGTCCCTGCCCTGGGGTCAATTACAG |
| | | | | AGCGAGGTCTGAGCCGCAGAG |
| 20 | EPB41L3_19071 | 23136 | EPB41L3 | GGGACAGTGGGGCTGACGCGTGGCTTCGGCGCCGCGCGGTCTCCCGAATCCCGAGCCCCGCGCCCGG |
| | | | | CGCGGCCGGGGTCCCCAACCGCCCTCCCGCTCGCCGGGACCCCCAC |
| 21 | EPB41L3_19072 | 23136 | EPB41L3 | GCGTGGGCCCCCGCCGCAGCTCCGCGGAGCCTCGGTGTCTCCTGCAACAGGGGGCGGGGGGAACAGC |
| | | | | GGCGAGCAGCCCTGGG |
| 22 | EPHB6_bay | 2051 | EPHB6 | GGGTGTCCGACCCAAGCCGAGCCCGAGCCCGAGCCCAGGCAGGAGCTTTACAGACAGCCTCTTCCCTTC |
| | | | | CCACTTCCTGCAGGCGCCCCACGCGTGCGATCCTCCCGGCCAAGACCCGCG |
| 23 | ERBB3_18097 | 2065 | ERBB3 | GCCCAGCCAAGTCCGGCCCGGGGGCCCCTAGGCTAGGACATCGAGGCAAGAGCCACCTGAACCGCTGG |
| | | | | CGAATTGGTGGCTGCTGCGGCGACGGCAGCGGAGGTTGCAAATTGCAATC |
| 24 | ERBB3_18099 | 2065 | ERBB3 | AGGGAGCCCAGCCAAGTCCGGCCCGGGGGCCCCTAGGCTAGGACATCGAGGCAAGAGCCACCTGAACC |
| | | | | GCTGGCGAATTGGTGGCTGCTGCGGCGACGGCAGCGGAGGTTGCA |
| 25 | FBLN2_13328 | 2199 | FBLN2 | CAGAGCGGAGGAAGCTGCGGACCTGGGGTGGGGGAACCCGCCCGCGGACCCCTGGCCCCCACCCCGC |
| | | | | GCCGGCCTCTGTGCCCGCATCTG |
| 26 | FBN2_18150 | 2201 | FBN2 | TCGGAGTCCCACAGGGCAACGAAGCGCGGGTAGCGGCTGCGGAGCCGGGCGGAGGTGCGCGGGGCCG |
| | | | | GGGCGTGCGGCCAGCAAGAG |
| 27 | FBN2_18151 | 2201 | FBN2 | CTGGAGACCTCGACAGAGCGCCGGCCCCCTGACTGCCCGCGAAGCGAGACGCGGGGCGCCGGGTCTA |
| | | | | GCGCAGTGAGCGGCGAGGCGCGGCGGAGGTGCAGCCGGCAGCCC |
| 28 | FOX-L2 | 668 | FOXL2 | GCGACAGGCCTCCAGCAAGCAAGCGCGGGCGGCATCCGCAGTCTCCAGAAGTTTGAGACTTGGCCGTAA |
| | | | | GCGGACTCGTGCGCCCCAACTCTTTGCCGCGCCAGCGCCTGGAGCGGAGAG |
| 29 | Gst-Pi | 2950 | GSTP1 | CCCGGGGTGCAGCGGCCGCCGGGGCTGGGGCCGGCGGGAGTCCGCGGGACCCTCCAGAAGAGCGGC |
| | | | | CGGCGCCGTGACTCAGCACTGGGGC |
| 30 | HS3ST2_19130 | 9956 | HS3ST2 | ACGTAAGAGCCTGGGAGCGCCCGAGCCGCCCGGCTGCCCGGAGCCCCATCGCCTAGGACCGGGAGATG |
| | | | | CTGGAAATGCAACCGCCTGTTCCCCGAGGAGCC |
| 31 | HS3ST2_19131 | 9956 | HS3ST2 | GCTCCGGGGCTCGCTCTCOGGCAGGCCCGGGGAGAGGTGGGGTGACAATGGGTTGGGGTGCGCGCGT |
| | | | | GCCTCATAGGTGCGAGACAGAGCGAGCCG |
| 32 | IGFBP7_19196 | 3490 | IGFBP7 | CCTGCCGGCGCCGCCACCCCCGCACGGCTCGCCCTCGCCGCGGGCGCACATAGGGCAGCAGCCGCAC |
| | | | | GCGTCGCGGGTCTCGCCCAGCAGGCAGCCC |
| 33 | IGFBP7_19200 | 3490 | IGFBP7 | CGCCCATGGGCCGGTCACGCCGGGTGCCCGCTCACCCCCCGACGCCAGCAGGAGCGCGCGCGCAGGC |
| | | | | CCCGCGGGGCCGGGAGGGCGGCACGGGCGGGGCCCCCGTGCTCTCAC |
| 34 | IRF7_18346 | 3665 | IRF7 | AGCTGAGAACCGGACGGGGTGGGATCGAGGAGGGTGCGAAGCGCCACTGTTTAGGTTTCGCTTTCCCGG |
| | | | | GAGCCTGACCCGCC |
| 35 | JAM3 | 83700 | JAM3 | GGGACTACAAGCCGCGCCGCGCTGCCGCTGGCCCCTCAGCAACCCTCGACATGGCGCTGAGGCGGCCA |
| | | | | CCGCGACTCCGGCTCTGCGCTCG |
| 36 | LOX_18967 | 4015 | LOX | GCGCGCAGAGCTGCAAAGGCCCGAGCAGGAGCACGGTCCAGGCGAAGCGCATCACTCCTTTTGCCAGAT |
| | | | | TGACCCCGCTCGAGGAGGACGT |
| 37 | LOX_18977 | 4015 | LOX | GGCAGAGGCGAGGAGCTGTCCGCCTTGCACGTTTCCAATCGCATTACGTGAACAAATAGCTGAGGGGCG |
| | | | | GCCGGGCCAGAACGGCTTGTGTA |
| 38 | LY6D_18402 | 8581 | LY6D | GATGTCGTCTGGGAGCAGTGCGGGCCCCTGCATTGCCAAGGCCTTATAGGCACGGGCTGGGCGGGGGT |
| | | | | GGGCAGTCCGCCAGCCAGCGGCATTCTGC |
| 39 | LY6K | 54742 | LY6K | GCGGGGCTCCCCCTACCGGCCAGACCCGGGGAGAGGCGCGCGGAGGCTGCGAAGGTTCCAGAAGGGC |
| | | | | GGGGAGGGGGCGCCGCGCGCTGACCCTCCCTGGGCACCGCTG |
| 40 | MACF1_bay | 23499 | MACF1 | GCCTTCGCTGCCGCCACGGGCCCGTCTTCTTCCTCCTTCGGCTCCCAGGGTAAGGCGCGGGGCGCGGG |
| | | | | GTTGGATGCAGGCGCCCTGCCCGCTGCGC |
| 41 | MCAM | 4162 | MCAM | AGAATTCAGGCCGGCCTCTATCGCTTCCCAGAACGATTGCACCACTGCCGCTGCCGCCGGCCTGACACT |
| | | | | GCCTCAGCCTCAGTGCTGGCAGCTTTGGGAGAAGAACCCTGCGC |
| 42 | NEFH_18452 | 4744 | NEFH | GCCGGATGAAGCATTCGGGCGTTCCCACTGCGGAAGGGCGGGGATGGCTGTGACGCAGGCGTGCCCGC |
| | | | | CGTCGCCTGCAGGG |
| 43 | NID2_9093 | 22795 | NID2 | CCACTCCGCCCCCAGGGAGCTCCCGGGTCATCCTCTCATCCGGGCTGCCCCGCGGCCCCCAAGGAGCC |
| | | | | CCACCCCCGGGACCAAATGGCCCGCAAG |
| 44 | NID2_9094 | 22795 | NID2 | CCCCGTGTGGGAAGAGCTCGTCTGGGTGCAGCGCCGCGGCCCGCAACATTAGCAACGGCAGCAGCAGT |
| | | | | AGCACTGGTAACGACGACAGCACCGGCCGCCCGGCCACCCG |
| 45 | PCDHB15_10763 | 56121 | PCDHB15 | CCTTGGTCACCAGGTAGCCCGGCTCGGCGGCCCGCCCGGGGCATCAGCTCGGTGCAGGGCGCGGAGC |
| | | | | CGTTCTGCAGCGGGAACAGCACGAAGAGTG |
| 46 | PHACTR3_11692 | 116154 | PHACTR3 | TCACTCTGCGAGCGGCCCCGCGACACGAGGCAGCCGCTCCCGTCCTCCGACGCGGCCATGGGCCCGGC |
| | | | | CGGCGCGGGGGCAAGTTAGAGCGAGCCGCGTGGAATCAGAGCATCC |
| 47 | POMC | 5443 | POMC | GACCTGGGCGCCTCTGGCTCTCCGCGGTCCCGAGTTCTCGACAAACTTTCTGCGCCGACTGCGGCATGA |
| | | | | GAAGCC |
| 48 | PRKCA_18626 | 5578 | PRKCA | GGGCGCTGAGGCAGAAGAACGTGCACGAGGTGAAGGACCACAAATTCATCGCGCGCTTCTTCAAGCAGC |
| | | | | CCACCTTCTGCAGCCACTGCACCGACTTCATCTGGTAGGTGCCG |
| 49 | PSEN1_18648 | 5663 | PSEN1 | CCAGGCCGGAGGCCCCGCCCCCTTCCTCCTGGCTCCTCCCCTCCTCCGTGGGCCGGCCGCCAACGACG |
| | | | | CCAGAGCCGGAAATGACGACAACGGTGAGGGTTC |
| 50 | RRAD_18698 | 6236 | RRAD | GATGCTCCGGCCGAGGTCCCGCCGCAGCCCTCCCCCAGCCCCCAGGTCGCGGCGCCCTCACCCGGGAC |
| | | | | CCCTCCGGACCTGGCGCATCCATCTGCAGCCGCCC |
| 51 | SFRP1 | 6422 | SFRP1 | TGCAGCCTCCGGAGTCAGTgccgcgcgcccgccgccccgcgccTTCCTGCTCGCCGCACCTCCGGGAG |
| | | | | CCGGGGCGCACCCAGCCCGCAGCGCCGCCTCCCCGCCCGCGCCGCCTCCGACCGCAGG |
| 52 | SFRP1_9381 | 6422 | SFRP1 | TCCTGCTCGCCGCACCTCCGGGAGCCGGGGCGCACCCAGCCCGCAGCGCCGCCTCCCCGCCCGCGCC |
| | | | | GCCTCCGACCGCAGGCCGAGGGCCGCCAC |
| 53 | SOD3_18740 | 6649 | SOD3 | AGTACAGAGTGGGGAGCGCAGCGACGAAGAATGAACAGGGCCTCGTGAGGTCCCAAACACCCGTTTCG |
| | | | | GTGGCAGGAAAG |
| 54 | SOX17 | 64321 | SOX17 | CTGCGCCAGCCGCTTGCGCTCGTCCTTAGCCCACACCATGAAAGCGTTCATCGGCCGCCGGATACGGG |
| | | | | ACTCGCCCTTG |
| 55 | SULF2_bay | 55959 | SULF2 | GCCAGCCGAGTCCGGAGGCATCGGGAGGTCGAGAGCCGCCGGGACCCCAGCTCTGCGTTCACTGCCCC |
| | | | | GTCCGGAGCTG |
| 56 | TIMP3 | 7078 | TIMP3 | GCGCCGGAGGCCAAGGTTGCCCCGCACGGCCCGGCGGGCGAGCGAGCTCGGGCTGCAGCAGCCCCGC |
| | | | | CGGCGGCGCGCACGGCAACTTTGGAGAG |
| 57 | TJP2_18792 | 9414 | TJP2 | CGGGTCAGAGCACTGTCCGGTGGTGCCCAGGAGGAGTAGGAGCAGGAGCAGAAGCAGAAGCGGGGTCC |
| | | | | GGAGCTGCGCGCCTACGCGGGACCTGTGTCC |
| 58 | TJP2_18797 | 9414 | TJP2 | GACCTCACCGGGGAAACACCGCGGACAGTCGGGCCAGCAGCGCCCGGAGCTCACTCCAGGTCTCCAAA |
| | | | | CTTGCAGCACTTCCCAGAGCGCGCGCGCTCGGAGCGGGACCTGCTT |
| 59 | TRPV2_18803 | 51393 | TRPV2 | TTACCCCGCAGGCTGAGGCCAGGGCGTGGCGGCTGCTGGGATCCCGGAGCTTCTCAGTAGCAGGGGCT |
| | | | | GCGGGAGGAAGTGAAGCCGGGAGGGGCTGCCGGCGCTGACAGCAGAGGA |
| 60 | UCHL1 | 7345 | UCHL1 | GCTGCATCTTCGCGGAGCGCCCGGCAGAAATAGCCTAGGGAAGACGAAAAACAGCTAGCGGAGCCGCC |
| | | | | CAGGCTGCAGCTATAAAGCGCCGGCCAGACGCACTGTGAG |
| 61 | VVDR69_18844 | 164781 | WDR69 | GCCCAGGCTGTGGCCTAGGCCGTCGGTTCCCGGCCATGCCTAGCTCCTCTGAGGTCGCCCTTAGTGAG |
| | | | | GACACGAGGTGC |
| 62 | ZFP42_bay | 132625 | ZFP42 | GGGGCCCCCAGGCACCCGGCCCGCACGCAAACCCTCAGCCCGGGGCCCCCTGACCCCCGCGTTCACCC |
| | | | | CTCAGCCCGGCCGCCGGGCACTGCGCATC |
| 63 | ZNF442_bay | 79973 | ZNF442 | CCGGCCTTCAGTCCCCTCGGCCGCGGGGTGGGAGCTGGGGGCTGGGCCGGCAGCCGGGACCCCGGGC |
| | | | | GTCCTGTCCCGTTTCTGCGCAGCAACTG |
| 64 | ZNF655 | 79027 | ZNF655 | CCACCGAGAAGCGCCGGCCTCGGGGCTGTCTACAGCGGCCCGGGAGAGGCTGTGGTGGCCCCGAGCG |
| | | | | CGAGTGTGTAGGTGACAGGACAGCGGCCAGGCCCGCCCCTCCCCTCGGT |

### Example 2

### Final Selection of Assays for Base 5

Finally a total number of 80 different assays (62 different genes), comprising:
64 assays designed for detecting the methylation status of 49 cancer markers identified by the aforementioned strategy,
assays for known published markers, and
good performing assays for cancer markers from other in-house cancer projects, were retained for analysis.

Differential methylation was assessed using the Base 5 platform; genes were ranked based on the best selectivity (sensitivity and specificity) between human lung cancer tissue and normal lung tissue samples. The investigated genes were ACSL6, ALS2CL, APC2, ARTS-1, BEX1, BMP7, BNIP3, CBR3, CD248, CD44, CHD5, DLK1, DPYSL4, DSC2, EDNRB, EPB41L3, EPHB6, ERBB3, FBLN2, FBN2, FOXL2, GNAS, GSTP1, HS3ST2, HPN, IGFBP7, IRF7, JAM3, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, SULF2, TIMP3, TJP2, TRPV2, UCHL1, WDR69, ZFP42, ZNF442, ZNF655.

Primer and amplicon sequences for the 49 genes are summarized in Table 1 and 2. Primer and amplicon sequences for the remainder 13 genes are listed in Table 3 and Table 4.

TABLE 3
MSP Primer sequences

| | | | Sense primer sequence (5′-3′) | Antisense primer sequence (5′-3′) |
|---|---|---|---|---|
| Gene ID | Symbol | Assay | (SEQ ID NO: 285-299, respectively) | (SEQ ID NO: 300-314, respectively) |
| 51752 | ARTS-1 | ARTS-1_17861 | GTAGTGGCGAGATGACGGA | AACCGAAACCAAACAAACG |
| 664 | BNIP3 | BNIP3_13409 | AGTGTTTAGAGAGTTCGTCGGTT | CGTAACGAATAAACTACGCGAT |
| 1910 | EDNRB | EDNRB_3 | GTCGGGTGTTATATGGTGCGT | AAAAACAATCCTCGTCCGAAA |
| 2778 | GNAS | GNAS_18295 | TTTTGAGAGGTCGTTATCGTGT | TTACTCGAACTATTCCCCGATT |
| 3249 | HPN | HPN_18326 | CGTTAGGTAGGGAGGAGGC | AACGATAAAATAAAAACAACGACC |
| 4686 | NCBP1 | NCBP1_18440 | ATTTGGGTAGAAAAGTTCGTTC | CTCAATAATTTTCCCGACGAC |
| 26025 | PCDHGA12 | PCDHGA12_18516 | AACGATTTGGGGTTAGAGTTTC | TAACCAAACTACCGCTTTACGA |
| 5214 | PFKP | PFKP_18555 | TTTTCGTTATGGACGCGGA | ATAACCTTACCGACCCCGAA |
| 10857 | PGRMC1 | PGRMC1_9140 | CGTTCGTATAGAGTTCGGTAATGTC | CCTATAACTAAACGCGACGCAC |
| 5256 | PHKA2 | PHKA2_18567 | CGTTTTTGGTTTTGTTTTCGT | AACCTAATTCCCGCCCGTT |
| 5256 | PHKA2 | PHKA2_18576 | TTTAGTAGGTTTGGTCGAGGC | ACGCTAACCCCAAAATCCG |
| 5256 | PHKA2 | PHKA2_18579 | TATAGGTAAGGGGGCGGTTTC | GCGACTCTAAAAATTCCGCT |
| 11186 | RASSF1A | RASSF1A | GCGTTGAAGTCGGGGTTC | CCCGTACTTCGCTAACTTTAAACG |
| 9770 | RASSF2 | RASSF2_1 | TTAGAGGGGCGTAGGGTGC | GCCAAACTAAAATCCCAACGA |
| 6446 | SGK | SGK_18737 | CGTTGTAGGATTTTGGGGGTC | ACCCTTCTCCCGCTCGATA |

TABLE 4
MSP amplicon sequences

| | Amplicon Sequence (converted) (5′-3′) | Amplicon Sequence (not converted) (5′-3′) |
|---|---|---|
| Assay | (SEQ ID NO: 315-329 respectively) | (SEQ ID NO: 330-344, respectively) |
| ARTS-1_17861 | GTAGTGGCGAGATGACGGATATTTAGCGAGTTTA | GCAGTGGCGAGATGACGGACACCCAGCGAGTCCA |
| | ATGGGCGTCGAACGCGTTTAGGTTTGGTGGATTT | ATGGGCGTCGAACGCGTCTAGGCTTGGTGGACTTG |
| | GTTAGCGTTTGTTTGGTTTCGGTT | TCAGCGCCTGCCTGGCTTCGGTC |
| BNIP3_13409 | AGTGTTTAGAGAGTTCGTCGGTTTTATCGTTTTTT | AGTGCCCAGAGAGTCCGCCGGTCCCACCGCCCCTT |
| | TAAAGGAGAATTCGGTTTATCGTTCGTCGCGGCG | CAAAGGAGAACCCGGCCCACCGCCCGCCGCGGCG |
| | GCGATCGCGTAGTTTATTCGTTACG | GCGACCGCGCAGCCCACTCGTCACG |
| EDNRB_3 | GTCGGGTGTTATATGGTGCGTGATAATTTGTTTTT | GCCGGGTGTCACATGGTGCGTGATAACTTGCCCTT |
| | GATTTGGGTTTATTTGAAGAGCGTAGAATTTTAA | GATTTGGGTTCATTTGAAGAGCGTAGAACTCTAAC |
| | TAAATAAATAGTTTTTTGGGATTTGTTTTCGGACG | AAATAAACAGCCTTTTGGGACCTGTCCCCGGACGA |
| | AGGATTGTTTTT | GGACTGCCCCC |
| GNAS_18295 | TTTTGAGAGGTCGTTATCGTGTTATGGGCGTGCG | TTTTGAGAGGCCGCCACCGTGTTATGGGCGTGCGC |
| | TAATTGTTTTTACGGTAATAATATGTTAGGATAA | AACTGCCTCTACGGCAATAATATGTCAGGACAACG |
| | CGCGATATTTTTTTTGAAATCGGGGAATAGTTCG | CGATATCCCCCCTGAAATCGGGGAACAGCCCGAGC |
| | AGTAA | AA |
| HPN_18326 | CGTTAGGTAGGGAGGAGGCGGGGAGGGGTTGGT | CGCCAGGCAGGGAGGAGGCGGGGAGGGGCTGGCC |
| | TTTAGAAGTGCGTGTTTGAAGCGGTTAATGTGTG | CCAGAAGTGCGTGTCTGAAGCGGCCAATGTGTGCA |
| | TAAATTAGTAAGGAGGAGGGGTGCGGGGTCGTT | AATCAGCAAGGAGGAGGGGTGCGGGGCCGCTGCC |
| | GTTTTTATTTTATCGTT | CCCACCTCACCGCC |
| NCBP1_18440 | ATTTGGGTAGAAAAGTTCGTTCGTGACGTTATTA | ATTTGGGTAGAAAAGCTCGCTCGTGACGTCACCAA |
| | AGTTTCGGAAGTTTTTTGGCGTCGGCGTAAGGGT | GCTCCGGAAGTCTCCTGGCGTCGGCGCAAGGGCCG |
| | CGTCGGGAAAATTATTGAG | CCGGGAAAACCATTGAG |
| PCDHGA12_18516 | AACGATTTGGGGTTAGAGTTTCGGGAGTTGGCGG | AACGACCTGGGGCTAGAGCCCCGGGAGCTGGCGG |
| | AGCGCGGAGTTCGTATCGTTTTTAGAGGTAGGAC | AGCGCGGAGTCCGCATCGTCTCCAGAGGTAGGAC |
| | GTAGTTTTTTTTTTTGAATTCGTAAAGCGGTAGTT | GCAGCTTTTCTCTCTGAATCCGCAAAGCGGCAGCT |
| | TGGTTA | TGGTCA |
| PFKP_18555 | TTTTCGTTATGGACGCGGACGATTTTCGGGTTTTT | TCCTCGCCATGGACGCGGACGACTCCCGGGCCCCC |
| | AAGGGTTTTTTGCGGAAGTTTTTGGAGTATTTTTT | AAGGGCTCCTTGCGGAAGTTCCTGGAGCACCTCTC |
| | CGGGGTCGGTAAGGTTAT | CGGGGCCGGCAAGGCCAT |
| PGRMC1_9140 | CGTTCGTATAGAGTTCGGTAATGTCGAGGTTTTTT | CGCTCGCACAGAGCCCGGCAATGCCGAGGCCCTCC |
| | TAACGGGTCGGTTTGCGAGGAGTAAAAAAGGGG | CAACGGGTCGGTCTGCGAGGAGCAAAAAAGGGGT |
| | TTTAGAGGAGGGTAGCGCGTGCGTCGCGTTTAGT | TCAGAGGAGGGCAGCGCGTGCGTCGCGCTCAGCT |
| | TATAGG | ATAGG |
| PHKA2_18567 | CGTTTTTGGTTTTGTTTTCGTCGCGGAGCGGAATT | CCCAGCAGGCCTGGCCGAGGCGGGACCTTCGTCGC |
| | TTTTAAGTCGCGGTTTGAGGAGGAAGGAAAAGG | TCCAGCCCCCGTCCCCGCCCCCGCGCCTCCCCGCC |
| | GGGCGGTTCGGGAGAGTCGTTGCGAAATTAGTA | GCGCGGAGCTCTGGTTGGCTTGCTTTCCAACCGGA |
| | ACGGGCGGGAATTAGGTT | CTTTGGGGCTAGCGT |
| PHKA2_18576 | TTTAGTAGGTTTGGTCGAGGCGGGATTTTCGTCG | CCCAGCAGGCCTGGCCGAGGCGGGACCTTCGTCGC |
| | TTTTAGTTTTCGTTTTCGTTTTCGCGTTTTTTCGTC | TCCAGCCCCCGTCCCCGCCCCCGCGCCTCCCCGCC |
| | GCGCGGAGTTTTGGTTGGTTTGTTTTTTAATCGGA | GCGCGGAGCTCTGGTTGGCTTGCTTTCCAACCGGA |
| | TTTTGGGGTTAGCGT | CTTTGGGGCTAGCGT |
| PHKA2_18579 | TATAGGTAAGGGGGCGGTTTCGTTTCGCGTTTTG | CACAGGTAAGGGGGCGGCCCCGCCCCGCGCCCTG |
| | GAACGATTTTACGGTTTCGTTTATATTTTCGTTTT | GAACGACCTCACGGCCCCGCCCACATCCCCGCCCC |
| | TGGTTTTATTTTCGTCGTAGAGCGGAATTTTTAGA | TGGCCCCACCTCCGCCGCAGAGCGGAACCCTCAGA |
| | GTCGC | GTCGC |
| RASSF1A | GCGTTGAAGTCGGGGTTCGTTTTGTGGTTTCGTTC | GCGCTGAAGTCGGGGCCCGCCCTGTGGCCCCGCCC |
| | GGTTCGCGTTTGTTAGCGTTTAAAGTTAGCGAAG | GGCCCGCGCTTGCTAGCGCCCAAAGCCAGCGAAG |
| | TACGGG | CACGGG |
| RASSF2_1 | TTAGAGGGGCGTAGGGTGCGCGGGGGTCGTTGG | TCAGAGGGGCGCAGGGTGCGCGGGGGCCGTTGGC |
| | TTTTTCGGGTATTTTTTTTTTGCGGTTTTTTCGTTT | CCTCCGGGCACTTCCCCTTTGCGGTCTCCCCGCCCT |
| | TTTTTCGGAGTTGGTGTTTGAGGTCGTTGGGATTT | CCTTCGGAGCTGGTGCCTGAGGTCGCTGGGACCTC |
| | TAGTTTGGC | AGCCTGGC |
| SGK_18737 | CGTTGTAGGATTTTGGGGGTCGGACGGTGGGATA | CGCTGCAGGACCCTGGGGGCCGGACGGTGGGATA |
| | CGGTTAATTTTCGGGGAGATGTTGTGGTTTTTATC | CGGCCAATCTCCGGGGAGATGCTGTGGCTCTTACC |
| | GAGCGGGAGAAGGGT | GAGCGGGAGAAGGGT |

### Example 3

### Lightcycler

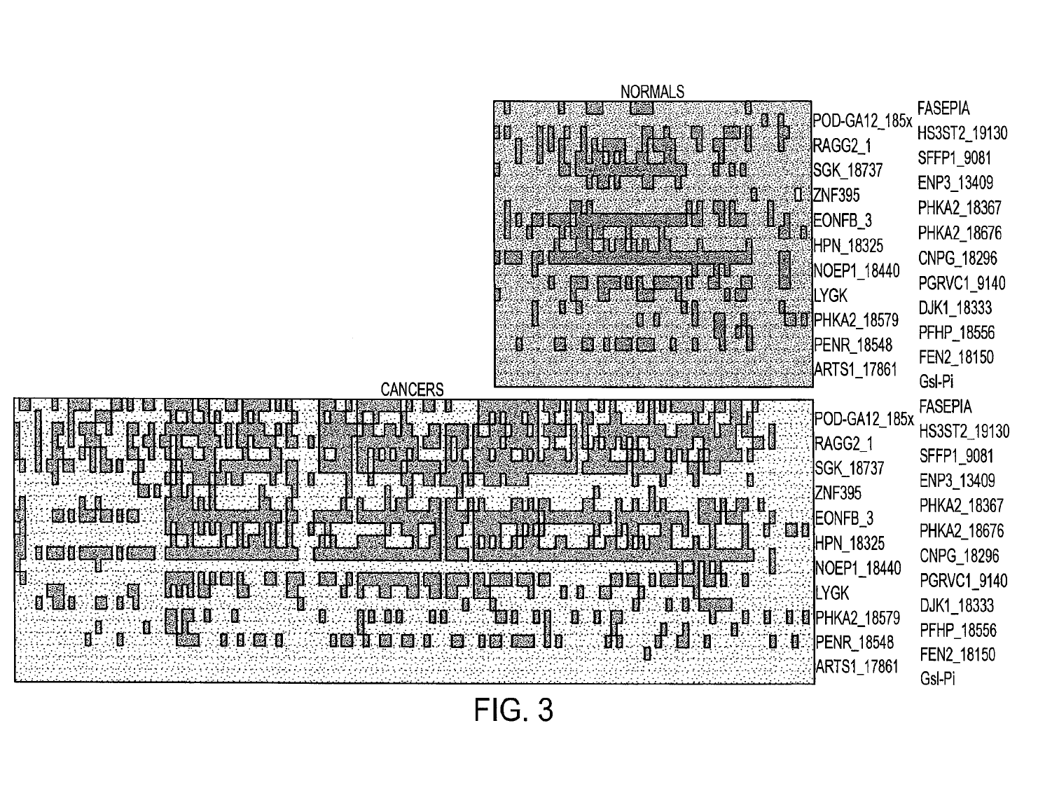

Twenty three assays issuing from the Base 5 analysis were selected and transferred to the Lightcycler platform in order to confirm the Base 5 results using 3 independent sample sets (JHU, Baltimore, USA; UMCG, Groningen, The Netherlands and Ulg, Liège, Belgium) and to define the best lung cancer methylation markers (Table 5). A beta-actin (ACTB) assay was included as an internal control. The assays were applied on a 384 well plate. The samples were randomized per plate. On this platform Ct values (cycle number at which the amplification curves cross the threshold value, set automatically by the software) and melting curves (Tm) were generated on the Roche LightCycler 480 using SYBR green as detector and for verification of the melting temperature. The size of the amplicon and intensity of the signal detected were analyzed using the Caliper LabChip electrophoretic separation system. Well-defined cut offs were set up on Ct, Tm, amplicon size and signal to get similar methylation calls when using the final Molecular Beacon (MB) detection system for further verification of the markers. DNA methylation calls were compared between 146 lung cancer and 58 normal tissue samples. DNA was isolated using proteinase K digestion and phenol/chloroform extraction method. DNA concentration was measured using NanoDrop Spectrophotometer. From each sample, up to 3 μg of genomic DNA was converted using a bisulphite based protocol (EZ DNA Methylation Kit™, ZYMO Research). After conversion and purification the equivalent of 20 ng of gDNA was used per reaction. An assay ranking was generated and the results are summarized in a methylation table (FIG. 3).

A sample was considered methylated if Ct is under 40 and if Tm and amplicon size are within the boundaries of Tm+/−2 degrees and amplicon size+/−10 bp. The intensity of the band detected by capillary electrophoresis had to be higher than 20. Those cut offs were set up to get similar methylation calls after Lightcycler analysis and real time PCR with Beacon detection system.

DNA methylation calls were compared between lung cancer tissue and normal lung tissue. An assay ranking with the set of samples was generated and the results are summarized in a methylation table (FIG. 3). A one-tailed Fisher's exact test was used as a scoring function to rank the candidate markers. The calculation of Fisher's exact test was based on a formula as described by Haseeb Ahmad Khan in “A visual basic software for computing Fisher's exact probability” (Journal of Statistical Software, vol. 08, issue i21, 2003).

A general overview of the ranking is given in Table 6.

TABLE 5
The 23 selected assays which were applied on the Lightcycler platform

| N^{o} | Assays |
|---|---|
| 1 | ARTS-1_17861 |
| 2 | BNIP3_13409 |
| 3 | DLK1_18033 |
| 4 | EDNRB_3 |
| 5 | FBN2_18150 |
| 6 | GNAS_18295 |
| 7 | GSTP1 |
| 8 | HPN_18326 |
| 9 | HS3ST2_19130 |
| 10 | LY6K |
| 11 | NCBP1_18440 |
| 12 | PCDHGA12_18516 |
| 13 | PFKP_18555 |
| 14 | PGRMC1_9140 |
| 15 | PHKA2_18567 |
| 16 | PHKA2_18576 |
| 17 | PHKA2_18579 |
| 18 | PSEN1_18648 |
| 19 | RASSF1A |
| 20 | RASSF2_1 |
| 21 | SFRP1_9381 |
| 22 | SGK_18737 |
| 23 | ZNF655 |

TABLE 6
Overview of the ranking of the assays tested on the Lightcycler platform

| | Ranking | | | | | | | |
|---|---|---|---|---|---|---|---|---|
| | 1 | 2 | 3 | 4 | 5 | 6 | 7 | 8 |
| Assays | RASSF1A | PCDHGA12_18516 | HS3ST2_19130 | RASSF2_1 | SFRP1_9381 | SGK_18737 | BNIP3_13409 | ZNF655 |
| Sens | 53.1 | 26.9 | 60.0 | 59.3 | 43.4 | 64.1 | 25.5 | 10.3 |
| Spec | 82.8 | 96.6 | 70.7 | 65.5 | 75.9 | 55.2 | 87.9 | 98.2 |
| Cncr | 77 | 39 | 87 | 86 | 63 | 93 | 37 | 15 |
| test+ | | | | | | | | |
| Cncr | 69 | 107 | 59 | 60 | 83 | 53 | 109 | 131 |
| test− | | | | | | | | |
| Nrml | 10 | 2 | 17 | 20 | 14 | 26 | 7 | 1 |
| test+ | | | | | | | | |
| Nrml | 48 | 56 | 41 | 38 | 44 | 32 | 51 | 56 |
| test− | | | | | | | | |
| p-value | 1.89E−06 | 4.31E−05 | 7.80E−05 | 1.32E−03 | 8.08E−03 | 1.07E−02 | 2.59E−02 | 3.19E−02 |
| (Fisher test) | | | | | | | | |

| | Ranking | | | | | | |
|---|---|---|---|---|---|---|---|
| | 9 | 10 | 11 | 12 | 13 | 14 | 15 |
| Assays | PHKA2_18567 | EDNRB_3 | PHKA2_18576 | HPN_18326 | GNAS_18295 | NCBP1_18440 | PGRMC1_9140 |
| Sens | 29.0 | 73.8 | 30.3 | 30.3 | 84.1 | 4.8 | 44.1 |
| Spec | 82.8 | 37.9 | 77.6 | 77.6 | 22.4 | 91.4 | 62.1 |
| Cncr | 42 | 107 | 44 | 44 | 122 | 7 | 64 |
| test+ | | | | | | | |
| Cncr | 104 | 39 | 102 | 102 | 24 | 139 | 82 |
| test− | | | | | | | |
| Nrml | 10 | 36 | 13 | 13 | 45 | 5 | 22 |
| test+ | | | | | | | |
| Nrml | 48 | 22 | 45 | 45 | 13 | 53 | 36 |
| test− | | | | | | | |
| p-value | 6.10E−02 | 8.71E−02 | 1.75E−01 | 1.75E−01 | 2.04E−01 | 2.40E−01 | 2.71E−01 |
| (Fisher test) | | | | | | | |

| | Ranking | | | | | | | |
|---|---|---|---|---|---|---|---|---|
| | 16 | 17 | 18 | 19 | 20 | 21 | 22 | 23 |
| Assays | LY6K | DLK1_18033 | PHKA2_18579 | PFKP_18555 | PSEN1_18648 | FBN2_18150 | ARTS-1_17861 | GSTP1 |
| Sens | 23.4 | 13.8 | 18.6 | 7.6 | 26.9 | 0.7 | 0.0 | 0.0 |
| Spec | 81.0 | 89.7 | 82.8 | 93.1 | 72.4 | 100.0 | 100.0 | 100.0 |
| Cncr | 34 | 20 | 27 | 11 | 39 | 1 | 0 | 0 |
| test+ | | | | | | | | |
| Cncr | 112 | 126 | 119 | 135 | 107 | 145 | 146 | 146 |
| test− | | | | | | | | |
| Nrml | 11 | 6 | 10 | 4 | 16 | 0 | 0 | 0 |
| test+ | | | | | | | | |
| Nrml | 47 | 52 | 48 | 54 | 42 | 58 | 58 | 58 |
| test− | | | | | | | | |
| p-value | 3.18E−01 | 3.47E−01 | 5.04E−01 | 5.70E−01 | 6.23E−01 | 7.16E−01 | 1.00E+00 | 1.00E+00 |
| (Fisher test) | | | | | | | | |

A comparison between the results coming from the Base 5 and the Lightcycler platforms has been performed.

Most of the interesting assays discovered on the Base 5 platform were confirmed on the Lightcycler platform.

### Example 4

### QMSP

Nineteen genes (APC2, BMP7, BNIP3, DLK1, DPYSL4, GSTP1, HS3ST2, JAM3, LOX, LY6K, NID2, PCDHGA12, PGRMC1, PHKA2, RASSF1A, RASSF2, SFRP1, SOX17, SULF2), were further selected based on the ranking on the Base 5 and/or Lightcycler platforms (marker discovery). For these assays, qMSPs using molecular beacons as detection system were designed (3 designs are evaluated per assay) and tested on control samples (cell lines). Several parameters (background, dynamic of the curve, highest range in fluorescence between beginning of the amplification and plateau phase, etc) were checked. In this phase of assay development, PCR material was used for generating the standard curves (instead of plasmids).

These assays were further verified on lung tissue samples collected by Ulg (Liège, Belgium), VUmc (Amsterdam, The Netherlands), UMCG (Groningen, The Netherlands) and Durham VA Medical Center (Durham, N.C., USA) (normal PE tissue samples #60, cancer PE tissue samples #86 (adenocarcinoma #30, squamous cell carcinoma #15, large cell carcinoma #6, carcinoid #1, neuroendocrine #1, NSCLC #33)). DNA was isolated from the lung tissue samples using a phenol-chloroform procedure, quantified using the picogreen method and 1 μg of DNA was bisulphite treated using the ZYMO kit.

The primers and molecular beacons used for the different qMSPs are summarized in Table 7. The amplicons are summarized in Table 8. qMSPs were carried out in a total volume of 12 μl in 384 well plates in an ABI PRISM 7900HT instrument (Applied Biosystems). The final reaction mixture consisted of in-house qMSP buffer (including 80.4 nmol of MgCl2), 60 nmol of each dNTPs, 0.5 U of Jump Start Taq polymerase (SIGMA), 72 ng of forward primer, 216 ng of reverse primer, 1.92 pmol of molecular beacon, 6.0 pmol of ROX (passive reference dye) and 50 ng of bisulphite converted genomic DNA. Thermal cycling was initiated with an incubation step of 5 minutes at 95° C., followed by 45 cycles (95° C. for 30 seconds, 57° C. for 30 seconds, 72° C. for 30 seconds). The last step was performed at 72° C. for 5 minutes. These conditions were similar for all the test genes as well as for ACTB.

Ct values were determined using the SDS software (version 2.2.2) supplied by Applied Biosystems with automatic baseline settings and threshold. The slopes and R² values for the different standard curves were determined after exporting data into excel.

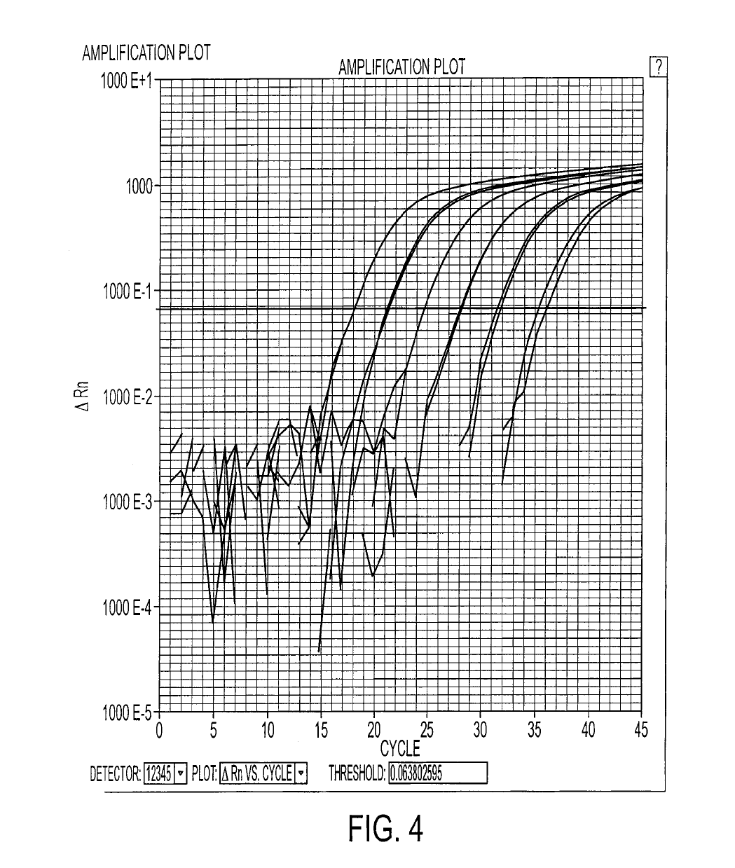

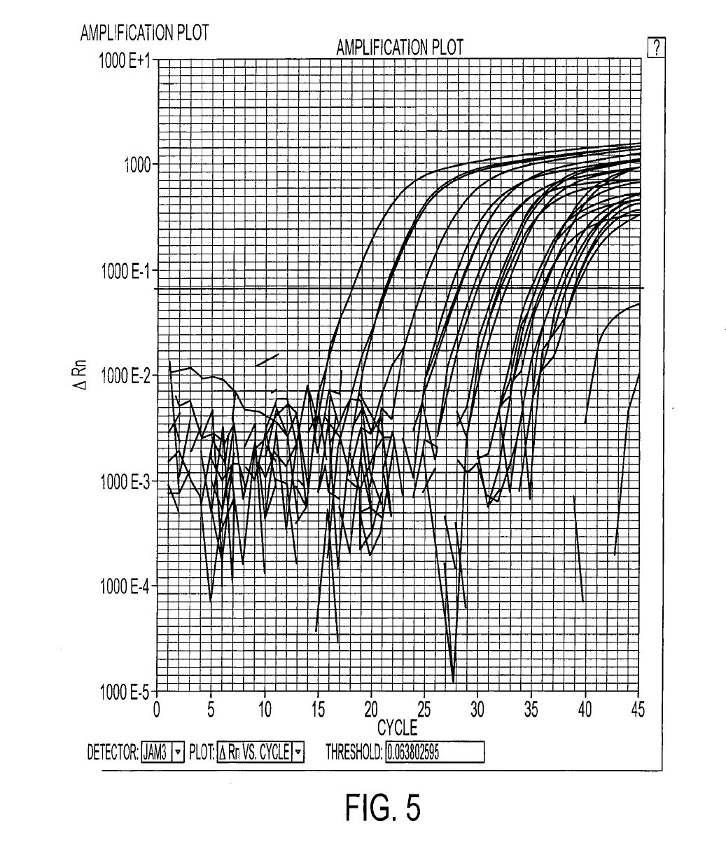

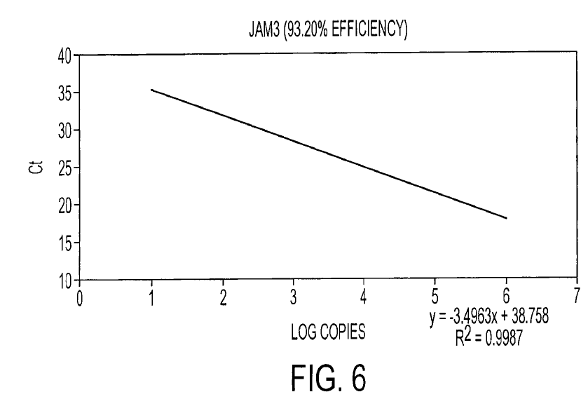

As an example, FIG. 4 shows the amplification plot for JAM3 obtained for the standard curve (960000 copies to 9.6 copies of the gene) and FIG. 5 shows the amplification plot for JAM3 obtained for the standard curve and for some samples. The Ct values plotted against the Log Copies of JAM3 (FIG. 6) give a R² of 0.9987 and the efficiency of the reaction is 93.20%.

In addition to the test genes, the independent reference gene ACTB was also measured.

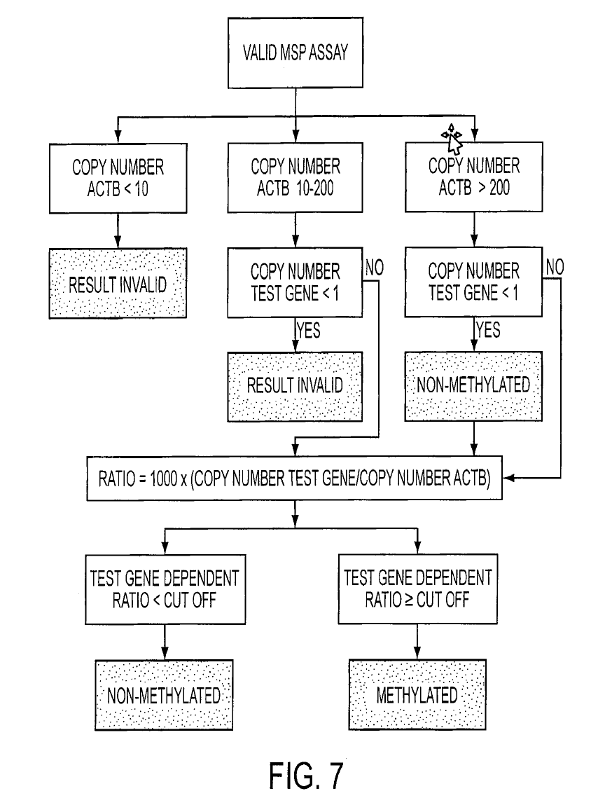

The ratios between the test genes and ACTB were calculated to generate the test result. The samples were classified as methylated, unmethylated, or invalid based on the decision tree shown in FIG. 7.

TABLE 7
qMSP primers and molecular beacons sequences

| | | | Sense primer sequence | Antisense primer | Molecular Beacon (5′-3′) |
|---|---|---|---|---|---|
| | | | (5′-3′) (SEQ ID | sequence (5′-3′) | (modification beacons: 5′ FAM, 3′ |
| Gene | | | NO: 345-365, | (SEQ ID NO: 366- | DABCYL) (SEQ ID NO: 387-407, |
| ID | Symbol | Assay | respectively) | 386, respectively) | respectively) |
| 10297 | APC2 | APC2 | TTATATGTCGGTTAC | GAACCAAAACGCTC | CGTCTGCCCCGTCGAAAACCCG |
| | | | GTGCGTTTATAT | CCCAT | CCGATTAACGCAGACG |
| 655 | BMP7 | BMP7_17911 | AGCGTAGAGATAGG | AAAACGATAACCCT | CGACATGCGCGGAGGGGTTAG |
| | | | TTGGTAACG | TAAACCGA | CGTGGTTGCATGTCG |
| 664 | BNIP3 | BNIP3 | TACGCGTAGGTTTTA | TCCCGAACTAAACG | CGACATGCCTACGACCGCGTC |
| | | | AGTCGC | AAACCCCG | GCCCATTAGCATGTCG |
| 8788 | DLK1 | DLK1_68536 | AAAGTTAGTAGGAG | AATACGACGCCAAA | CGACATGCGGGCGGTCGGGGT |
| | | | TAAGAGGACGC | AACCG | CGCGCATGTCG |
| 10570 | DPYSL4 | DPYSL4_18050 | GGGGTTATAGTTTGG | GCTCTAAAAACCAC | CGACATGCGGTTCGGGTTATTT |
| | | | CGTTC | ACCCGTC | GCGAAGGAGTCGGCATGTCG |
| 2950 | GSTP1 | GSTPTi current | TTCGGGGTGTAGCG | GCCCCAATACTAAA | CGTCTGCTTGGGGTCGGCGGG |
| | | | GTCGTC | TCACGACG | AGTTCGCGGGATTGCAGACG |
| 9956 | HS3ST2 | HS3ST2_2 | GTTTCGGGGTTCGTT | CGACTCGCTCTATCT | CGACATGCACGCGCGCACCCC |
| | | | TTTC | CGCAC | AACCCAGCATGTCG |
| 9956 | HS3ST2 | HS3ST2_8 | AGTTTTCGGAGAAG | ACGACTAAACTACT | CGACATGCACCACGACCACGC |
| | | | ACGGC | ATAACCCTACGA | GAATCGAACGCATGTCG |
| 9956 | HS3ST2 | HS3ST2_nor | CGTTAGGTTATTTTT | CGACTATACGAACT | CGACATGCACGCCGACCGCGA |
| | | | TAAATAGAGTCGGT | AACGAATAAACCG | TCTAACTCGCATGTCG |
| | | | AGC | | |
| 83700 | JAM3 | JAM3 | GGGATTATAAGTCG | CGAACGCAAAACCG | CGACACGATATGGCGTTGAGG |
| | | | CGTCGC | AAATCG | CGGTTATCGTGTCG |
| 4015 | LOX | LOX_25068 | CGTGAATAAATAGT | GACAATCCCGAAAA | CGTCTGCCACAAACCGTTCTAA |
| | | | TGAGGGGC | ACGAAC | CCCGACCGCGCAGACG |
| 54742 | LY6K | LY6K | GCGGGGTTTTTTTTA | CAACGATACCCAAA | CGACATGCCGACGCCCCCTCCC |
| | | | TCGGTTAGATTC | AAAAATCAACGCG | CGGCATGTCG |
| 22795 | NID2 | NID2_9091 | GCGGTTTTTAAGGA | CTACGAAATTCCOFT | CGACATGGGTTCGTAAGGTTTG |
| | | | GTTTTATTTTC | TACGCT | GGGTAGCGGCCATGTCG |
| 26025 | PCDHGA12 | PCDHGA12_18516 | AACGATTTGGGGTT | TAACCAAACTACCG | CGACATGCGCGCTCCGCCAACT |
| | | | AGAGTTTC | CTTTACGA | CCGCATGTCG |
| 10857 | PGRMC1 | PGRMC1_9140 | CGTTCGTATAGAGTT | CCTATAACTAAACG | CGACATGCGGGGTTTAGAGGA |
| | | | CGGTAATGTC | CGACGCAC | GGGTAGCGCGCATGTCG |
| 5256 | PHKA2 | PHKA2_70210 | TCGTCGTTTTAGTTT | ACGCTAACCCCAAA | ACTCCCGCGTTTTTTCGTCGCGC |
| | | | TCGTTTTC | ATCCG | GGAGT |
| 11186 | RASSF1A | RASSF1A | GCGTTGAAGTCGGG | CCCGTACTTCGCTAA | CGTCTGCGTGGTTTCGTTCGGT |
| | | | GTTC | CTTTAAACG | TCGCGTTTGTTAGGCAGACG |
| 9770 | RASSF2 | RASSF2_2b | AGGTAGGTTTTAGTT | GACCTCAAACACCA | CGACATGCGGGTGCGCGGGGG |
| | | | TTCGGC | ACTCCG | TCGTTGGGCATGTCG |
| 6422 | SFRP1 | SFRP1 | TGTAGTTTTCGGAGT | CCTACGATCGAAAA | CGACATGCTCGGGAGTCGGGG |
| | | | TAGTGTCGCGC | CGACGCGAACG | CGTATTTAGTTCGTAGCGGCAT |
| | | | | | GTCG |
| 64321 | SOX17 | SOX17_66072 | GAGATGTTTCGAGG | CCGCAATATCACTA | CGACATGCGTTCGTGTTTTGGT |
| | | | GTTGC | AACCGA | TTGTCGCGGTTTGGCATGTCG |
| 55959 | SULF2 | SULF2_Bay | GTTAGTCGAGTTCGG | CAACTCCGAACGAA | CGACATGCCCGACGACTCTCG |
| | | | AGGTATC | ACAATAAACG | ACCTCCCGCATGTCG |

TABLE 8
qMSP amplicon sequences

| | Amplicon Sequence (converted) (5′-3′) | Amplicon Sequence (non converted) (5′-3′) |
|---|---|---|
| Assay | (SEQ ID NO: 408-428, respectively) | (SEQ ID NO: 429-449, respectively) |
| APC2 | TTATATGTCGGTTACGTGCGTTTATATTTAGTTAAT | CCACATGTCGGTCACGTGCGCCCACACCCAGCCAA |
| | CGGCGGGTTTTCGACGGGAATGGGGAGCGTTTTG | TCGGCGGGCTCCCGACGGGAATGGGGAGCGCCCT |
| | GTTC | GGTCC |
| BMP7_17911 | AGCGTAGAGATAGGTTGGTAACGGTTTTTAGGGAG | AGCGCAGAGACAGGCTGGCAACGGCTTCAGGGAG |
| | GCGCGGAGGGGTTAGCGTGGTTGGTTTAAAAGGA | GCGCGGAGGGGTCAGCGTGGCTGGCTTAAAAGGA |
| | TATAGGGATTGAGGGGTAAGATCGGTTTAAGGGT | TACAGGGACTGAGGGGCAAGACCGGCTCAAGGGT |
| | TATCGTTTT | CACCGCTTC |
| BNIP3 | TACGCGTAGGTTTTAAGTCGCGGTTAATGGGCGAC | CACGCGCAGGCCCCAAGTCGCGGCCAATGGGCGA |
| | GCGGTCGTAGATTCGTTCGGTTTCGTTTTGTTTTGT | CGCGGCCGCAGATCCGCCCGGCCCCGCCCTGCCCT |
| | GAGTTTTTTCGGTCGGGTTGCGGGGTTTCGTTTAG | GTGAGTTCCTCCGGCCGGGCTGCGGGGCTCCGCTC |
| | TTCGGGA | AGTCCGGGA |
| DLK1_68536 | AAAGTTAGTAGGAGTAAGAGGACGCGTAGGAGGG | AAAGCCAGCAGGAGCAAGAGGACGCGCAGGAGG |
| | TTTCGGTCGCGGTTATTTTTGGGCGGTCGGGGTCG | GCTTCGGTCGCGGTCATCTCTGGGCGGCCGGGGTC |
| | CGGTTTCGGGAGCGGTGCGGGCGCGGGTTCGGTTT | GCGGTCCCGGGAGCGGTGCGGGCGCGGGTCCGGC |
| | TTGGCGTCGTATT | TCCTGGCGCCGCACT |
| DPYSL4_18050 | GGGGTTATAGTTTGGCGTTCGGATTTTGGTTCGGG | GGGGTCACAGCCTGGCGCTCGGACCCTGGCCCGG |
| | TTATTTGCGAAGGAGTCGGTTTTGGTTAAGGTGTT | GTCATCTGCGAAGGAGCCGGCTTTGGCCAAGGTG |
| | TTTTTGGACGGGTGTGGTTTTTAGAGC | CCTTCCTGGACGGGTGTGGTTCCCAGAGC |
| GSTPi current | TTCGGGGTGTAGCGGTCGTCGGGGTTGGGGTCGGC | CCCGGGGTGCAGCGGCCGCCGGGGCTGGGGCCGG |
| | GGGAGTTCGCGGGATTTTTTAGAAGAGCGGTCGG | CGGGAGTCCGCGGGACCCTCCAGAAGAGCGGCCG |
| | CGTCGTGATTTAGTATTGGGGC | GCGCCGTGACTCAGCACTGGGGC |
| HS3ST2_2 | GTTTCGGGGTTCGTTTTTCGGTAGGTTCGGGGAGA | GCTCCGGGGCTCGCTCTCCGGCAGGCCCGGGGAG |
| | GGTGGGGTGATAATGGGTTGGGGTGCGCGCGTGT | AGGTGGGGTGACAATGGGTTGGGGTGCGCGCGTG |
| | TTTATAGGTGCGAGATAGAGCGAGTCG | CCTCATAGGTGCGAGACAGAGCGAGCCG |
| HS3ST2_8 | AGTTTTCGGAGAAGACGGCGTTTTTAACGTTCGAT | AGCCCCCGGAGAAGACGGCGCCCCCAACGCCCGA |
| | TCGCGTGGTCGTGGTAGCGTTACGCGAGTTTTTTA | CCCGCGTGGCCGTGGCAGCGCCACGCGAGCCCTCT |
| | GGCGATCGTAGGGTTATAGTAGTTTAGTCGT | AGGCGACCGCAGGGCCACAGCAGCTCAGCCGC |
| HS3ST2_nor | CGTTAGGTTATTTTTTAAATAGAGTCGGTAGCGCG | CGTCAGGCCACTCCTTAAATAGAGCCGGCAGCGC |
| | TTTCGTTCGGTATTTTTCGAAGAGTTAGATCGCGG | GCTCCGCTCGGCATTTCCCGAAGAGCCAGATCGCG |
| | TCGGCGTTAGCGTTATCGTTCGGTTTATTCGTTAGT | GCCGGCGCCAGCGCCACCGTCCGGTCCACCCGCC |
| | TCGTATAGTCG | AGCCCGCACAGCCG |
| JAM3 | GGGATTATAAGTCGCGTCGCGTTGTCGTTGGTTTT | GGGACTACAAGCCGCGCCGCGCTGCCGCTGGCCC |
| | TTAGTAATTTTCGATATGGCGTTGAGGCGGTTATC | CTCAGCAACCCTCGACATGGCGCTGAGGCGGCCA |
| | GCGATTTCGGTTTTGCGTTCG | CCGCGACTCCGGCTCTGCGCTCG |
| LOX_25068 | CGTGAATAAATAGTTGAGGGGCGGTCGGGTTAGA | CGTGAACAAATAGCTGAGGGGCGGCCGGGCCAGA |
| | ACGGTTTGTGTAATTTTGTAAACGTGTTAGAAAGT | ACGGCTTGTGTAACTTTGCAAACGTGCCAGAAAGT |
| | TTAAAATTTTTTTTTTTTTTTTTATTTTAGATATTGT | TTAAAATCTCTCCTCCTTCCTTCACTCCAGACACTG |
| | TCGTTTTTCGGGATTGTC | CCCGCTCTCCGGGACTGCC |
| LY6K | GCGGGGTTTTTTTTATCGGTTAGATTCGGGGAGAG | GCGGGGCTCCCCCTACCGGCCAGACCCGGGGAGA |
| | GCGCGCGGAGGTTGCGAAGGTTTTAGAAGGGCGG | GGCGCGCGGAGGCTGCGAAGGTTCCAGAAGGGCG |
| | GGAGGGGGCGTCGCGCGTTGATTTTTTTTGGGTAT | GGGAGGGGGCGCCGCGCGCTGACCCTCCCTGGGC |
| | CGTTG | ACCGCTG |
| NID2_9091 | GCGGTTTTTAAGGAGTTTTATTTTCGGGATTAAAT | GCGGCCCCCAAGGAGCCCCACCCCCGGGACCAAA |
| | GGTTCGTAAGGTTTGGGGTAGCGGCGTTGTAGGA | TGGCCCGCAAGGTTTGGGGCAGCGGCGTTGCAGG |
| | GATGAGTTTAGCGTAAAGGGAATTTCGTAG | AGATGAGCTCAGCGCAAAGGGAACCCCGCAG |
| PCDHGA12_18516 | AACGATTTGGGGTTAGAGTTTCGGGAGTTGGCGG | AACGACCTGGGGCTAGAGCCCCGGGAGCTGGCGG |
| | AGCGCGGAGTTCGTATCGTTTTTAGAGGTAGGACG | AGCGCGGAGTCCGCATCGTCTCCAGAGGTAGGAC |
| | TAGTTTTTTTTTTTGAATTCGTAAAGCGGTAGTTTG | GCAGCTTTTCTCTCTGAATCCGCAAAGCGGCAGCT |
| | GTTA | TGGTCA |
| PGRMC_19140 | CGTTCGTATAGAGTTCGGTAATGTCGAGGTTTTTT | CGCTCGCACAGAGCCCGGCAATGCCGAGGCCCTC |
| | TAACGGGTCGGTTTGCGAGGAGTAAAAAAGGGGT | CCAACGGGTCGGTCTGCGAGGAGCAAAAAAGGGG |
| | TTAGAGGAGGGTAGCGCGTGCGTCGCGTTTAGTTA | TTCAGAGGAGGGCAGCGCGTGCGTCGCGCTCAGC |
| | TAGG | TATAGG |
| PHKA2_70210 | TCGTCGTTTTAGTTTTCGTTTTCGTTTTCGCGTTTTT | TCGTCGCTCCAGCCCCCGTCCCCGCCCCCGCGCCT |
| | TCGTCGCGCGGAGTTTTGGTTGGTTTGTTTTTTAAT | CCCCGCCGCGCGGAGCTCTGGTTGGCTTGCTTTCC |
| | CGGATTTTGGGGTTAGCGT | AACCGGACTTTGGGGCTAGCGT |
| RASSF1A | GCGTTGAAGTCGGGGTTCGTTTTGTGGTTTCGTTC | GCGCTGAAGTCGGGGCCCGCCCTGTGGCCCCGCCC |
| | GGTTCGCGTTTGTTAGCGTTTAAAGTTAGCGAAGT | GGCCCGCGCTTGCTAGCGCCCAAAGCCAGCGAAG |
| | ACGGG | CACGGG |
| RASSF2_2b | AGGTAGGTTTTAGTTTTCGGCGCGGGGAGGCGGC | AGGCAGGTCCCAGTCCCCGGCGCGGGGAGGCGGC |
| | GCGTTTTAGAGGGGCGTAGGGTGCGCGGGGGTCG | GCGCTTCAGAGGGGCGCAGGGTGCGCGGGGGCCG |
| | TTGGTTTTTCGGGTATTTTTTTTTTGCGGTTTTTTCG | TTGGCCCTCCGGGCACTTCCCCTTTGCGGTCTCCCC |
| | TTTTTTTTCGGAGTTGGTGTTTGAGGTC | GCCCTCCTTCGGAGC |
| SFRP1 | TGTAGTTTTCGGAGTTAGTGTCGCGCGTTCGTCGT | TGCAGCCTCCGGAGTCAGTGCCGCGCGCCCGCCGC |
| | TTCGCGTTTTTTTGTTCGTCGTATTTTCGGGAGTCG | CCCGCGCCTTCCTGCTCGCCGCACCTCCGGGAGCC |
| | GGGCGTATTTAGTTCGTAGCGTCGTTTTTTCGTTCG | GGGGCGCACCCAGCCCGCAGCGCCGCCTCCCCGC |
| | CGTCGTTTTCGATCGTAGG | CCGCGCCGCCTCCGACCGCAGG |
| SOX17_66072 | GAGATGTTTCGAGGGTTGCGCGGGTTTTTCGGTTC | GAGATGCCCCGAGGGCTGCGCGGGTCTCCCGGCC |
| | GAAGTCGTCGTTCGTGTTTTGGTTTGTCGCGGTTTG | CGAAGCCGCCGCCCGTGTTCTGGCCTGTCGCGGTC |
| | GTTTATAGCGTATTTAGGGTTTTTAGTCGGTTTAGT | TGGTCTACAGCGTACCCAGGGCCCCCAGCCGGCCT |
| | GATATTGCGG | AGTGACACTGCGG |
| SULF2_Bay | GTTAGTCGAGTTCGGAGGTATCGGGAGGTCGAGA | GCCAGCCGAGTCCGGAGGCATCGGGAGGTCGAGA |
| | GTCGTCGGGATTTTAGTTTTGCGTTTATTGTTTCGT | GCCGCCGGGACCCCAGCTCTGCGTTCACTGCCCCG |
| | TCGGAGTTG | TCCGGAGCTG |

The highest methylation value of the normal tissue specimens was taken as a directive to define a cut off above which the cases were considered to be methylated. The analytical cut off was finally set to give the highest possible specificity and/or above 3 times STDEV (Normal) (excluding outliers).

The one-tailed Fisher's exact test as described above was used as a scoring function to rank the candidate markers (Journal of Statistical Software, vol. 08, issue i21, 2003).

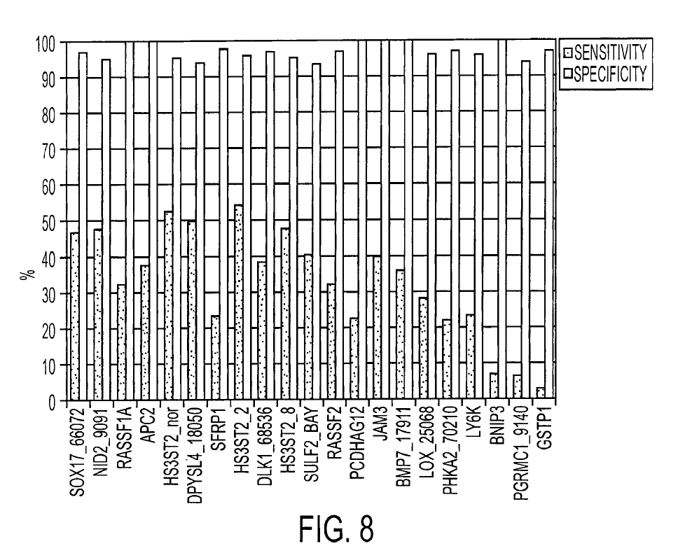

Table 9 summarizes the results obtained for JAM3. Table 10 summarizes the results obtained for all the tested markers on tissue samples. The individual performances of the assays are shown in FIG. 8 and the assays are ranked according their p-value (Fisher's exact test). The best performing markers were further tested on clinical samples (sputum samples).

TABLE 9
Summary of the test results for JAM3 on lung tissue samples. The black boxes indicate
the methylated results; grey boxes indicate the unmethylated results.

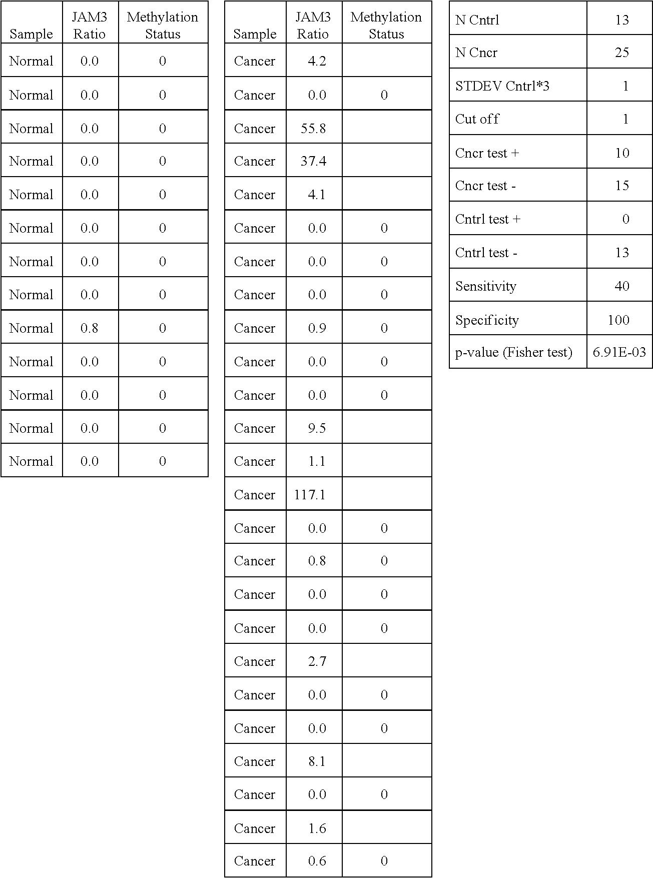

TABLE 10
Summary of the results obtained for all the tested markers on lung tissue samples.

| | qMSP ranking | | | | | | | |
|---|---|---|---|---|---|---|---|---|
| | 1 | 2 | 3 | 4 | 5 | 6 | 7 | 8 |
| Assays | SOX17_66072 | NID2_9091 | RASSF1A | APC2 | HS3ST2_nor | DPYSL4_18050 | SFRP1 | HS3ST2_2 |
| STDEV | 12 | 117 | 1 | 53 | 16 | 62 | 12 | 23 |
| Cntrl*3 | | | | | | | | |
| Cut off | 15 | 15 | 5 | 100 | 15 | 30 | 10 | 10 |
| Cncr | 36 | 37 | 27 | 13 | 17 | 16 | 20 | 12 |
| test+ | | | | | | | | |
| Cncr | 40 | 40 | 56 | 21 | 15 | 16 | 64 | 10 |
| test− | | | | | | | | |
| Cntrl | 1 | 2 | 0 | 0 | 1 | 2 | 1 | 1 |
| test+ | | | | | | | | |
| Cntrl | 44 | 43 | 55 | 37 | 23 | 31 | 56 | 24 |
| test− | | | | | | | | |
| Sensitivity | 47 | 48 | 33 | 38 | 53 | 50 | 24 | 55 |
| Specificity | 98 | 96 | 100 | 100 | 96 | 94 | 98 | 96 |
| p-value | 1.61E−08 | 1.09E−07 | 1.41E−07 | 1.60E−05 | 6.62E−05 | 6.76E−05 | 1.16E−04 | 1.18E−04 |
| (Fisher test) | | | | | | | | |

| | qMSP ranking | | | | | | |
|---|---|---|---|---|---|---|---|
| | 9 | 10 | 11 | 12 | 13 | 14 | 15 |
| Assays | DLK1_68536 | HS3ST2_8 | SULF2_Bay | RASSF2 | PCDHAG12 | JAM3 | BMP7_17911 |
| STDEV | 3 | 11 | 75 | 1 | 0 | 1 | 4 |
| Cntrl*3 | | | | | | | |
| Cut off | 0 | 10 | 10 | 2 | 0 | 1 | 5 |
| Cncr | 12 | 11 | 13 | 10 | 7 | 10 | 9 |
| test+ | | | | | | | |
| Cncr | 19 | 12 | 19 | 21 | 24 | 15 | 16 |
| test− | | | | | | | |
| Cntrl | 1 | 1 | 2 | 1 | 0 | 0 | 0 |
| test+ | | | | | | | |
| Cntrl | 32 | 23 | 31 | 32 | 34 | 13 | 13 |
| test− | | | | | | | |
| Sensitivity | 39 | 48 | 41 | 32 | 23 | 40 | 36 |
| Specificity | 97 | 96 | 94 | 97 | 100 | 100 | 100 |
| p-value | 3.70E−04 | 6.47E−04 | 9.62E−04 | 2.08E−03 | 3.78E−03 | 6.91E−03 | 1.25E−02 |
| (Fisher test) | | | | | | | |

| | qMSP ranking | | | | | |
|---|---|---|---|---|---|---|
| | 16 | 17 | 18 | 19 | 20 | 21 |
| Assays | LOX_25068 | PHKA2_70210 | LY6K | BNIP3 | PGRMC1_9140 | GSTP1 |
| STDEV | 4 | 39 | 152 | 0 | 316 | 1 |
| Cntrl*3 | | | | | | |
| Cut off | 5 | 40 | 155 | 1 | 250 | 2 |
| Cncr | 7 | 7 | 7 | 2 | 2 | 1 |
| test+ | | | | | | |
| Cncr | 18 | 25 | 23 | 28 | 29 | 30 |
| test− | | | | | | |
| Cntrl | 1 | 1 | 1 | 0 | 2 | 1 |
| test+ | | | | | | |
| Cntrl | 25 | 33 | 24 | 25 | 32 | 33 |
| test− | | | | | | |
| Sensitivity | 28 | 22 | 23 | 7 | 6 | 3 |
| Specificity | 96 | 97 | 96 | 100 | 94 | 97 |
| p-value | 2.13E−02 | 2.18E−02 | 4.66E−02 | 2.93E−01 | 6.58E−01 | 7.30E−01 |
| (Fisher test) | | | | | | |

### Example 5

### Best Performing Markers Tested on Sputum Samples

The control sputum samples were collected from the Lung Cancer Clinical Collaborative Research Agreement study of ONCO with the UMCG hospital (Groningen, The Netherlands). These samples were taken from participants to the NELSON screening program (a randomized controlled screening trial for lung cancer using multi-slice low-dose CT in high risk subjects—current smokers (55%) and former smokers (45%) who (had) smoked at least 16 cigarettes a day for at least 26 years or at least 11 cigarettes a day for at least 31 years).

The cancer sputum samples (stage IA #2, stage IIIA #3, stage IIIB #1, stage IV #1, stage unknown #1) were collected from the Lung Cancer Clinical Collaborative Research Agreement study of ONCO with Durham VA Medical Center (Durham, N.C., USA). Patients with histologically proven NSCLC or patients suspected of having NSCLC planning to undergo resection and who have a predicted probability of 75% or more of having NSCLC (e.g., using nomograms such as at the worldwide web domain chestx-ray.com, at the page SPN/SPNProb.html) were included in the study.

Subjects were provided with a sterile cup containing Saccomanno's fixative and instructed to take a deep breath, cough deeply, and expectorate into the cup for 3 consecutive days. The samples were centrifuged at 1500×g for 15 min to sediment all cellular material, the supernatants were removed and the cell pellet was washed with PBS. DNA was extracted from the sputum cells using standard salt-chloroform extraction and ethanol precipitation for high molecular DNA and dissolved in 250 μL TE buffer (10 mM Tris; 1 mM EDTA (pH 8.0)). DNA was quantified using the picogreen method and 20 μg (or maximum amount if less than 20 μg recovered from DNA extraction) of DNA was bisulphite treated using the EpiTect bisulfite kit (QIAGEN).

QMSP was performed after bisulphite treatment on denatured genomic DNA. The assays were carried out as described above, except that 960 ng of bisulphite converted genomic DNA was added in the reaction mixture. The samples were classified as methylated, unmethylated, or invalid as described above. The results based on ratio (copy number gene tested/copy number ACTB) and based on copy number obtained for all the tested markers on sputum samples from lung cancer patients and from control patients were ranked according their p-value (Fisher's exact test) (Table 11—ratio, Table 12—copy number).

Several combinations of markers were investigated to maximize sensitivity of detection, without significantly compromising specificity. The samples were classified as methylated if at least one of the tested markers scored positive based on ratio or based on copy number. Examples of the performance of combination of markers are summarized in Table 13 (ratio) and in Table 14 (copy number). Specificity above 90% is obtained for some combinations of markers (based on ratio and copy number). Sensitivity of 100% is obtained for some combinations of markers (based copy number).

TABLE 11
Summary of the results based on ratio obtained for all the tested lung
markers on sputum samples from lung cancer patients and from control
patients (cncr: cancer; ctrl: control; AUC: area under curve).

| | RATIO | | | | |
|---|---|---|---|---|---|
| Assays | RASSF1A | SOX17_66072 | HS3ST2_nor | NID2_9091 | SFRP1 |
| 3 * STDEV Cntrl sputum | 0 | 8 | 6 | 5 | 7 |
| Cut off ratio | 0 | 8 | 6 | 5 | 7 |
| Sputum Cncr test+ | 4 | 3 | 1 | 1 | 0 |
| Sputum Cncr test− | 4 | 5 | 7 | 7 | 8 |
| Sputum Cntrl test+ | 1 | 1 | 1 | 1 | 1 |
| Sputum Cntrl test− | 26 | 26 | 26 | 26 | 26 |
| p-value (Fisher test) | 5.99E−03 | 3.02E−02 | 4.10E−01 | 4.10E−01 | 7.71E−01 |
| sensitivity | 50% | 38% | 13% | 13% | 0% |
| specificity | 96% | 96% | 96% | 96% | 96% |
| AUC (ROC analysis) | 0.750 | 0.769 | 0.767 | 0.687 | 0.514 |
| 95% CI | 0.572 to 0.882 | 0.593 to 0.895 | 0.591 to 0.894 | 0.506 to 0.835 | 0.338 to 0.689 |

TABLE 12
Summary of the results based on copy number obtained for all the tested lung markers
on sputum samples from lung cancer patients and from control patients (cncr: cancer; ctrl:
control; AUC: area under curve).

| | COPY NUMBER | | | | |
|---|---|---|---|---|---|
| Assays | NID2_9091 | SOX17_66072 | HS3ST2_nor | RASSF1A | SFRP1 |
| 3 * STDEV Cntrl sputum | 1804 | 793 | 323 | 2 | 164 |
| Cut off sputum | 300 | 600 | 300 | 0 | 150 |
| Sputum Cncr test+ | 6 | 6 | 6 | 4 | 1 |
| Sputum Cncr test− | 2 | 2 | 2 | 4 | 7 |
| Sputum Cntrl test+ | 1 | 1 | 2 | 1 | 2 |
| Sputum Cntrl test− | 26 | 26 | 25 | 26 | 25 |
| p-value (Fisher test) | 1.14E−04 | 1.14E−04 | 4.27E−04 | 5.99E−03 | 4.29E−01 |
| Sensitivity | 75% | 75% | 75% | 50% | 13% |
| Specificity | 96% | 96% | 93% | 96% | 93% |
| AUC (ROC analysis) | 0.765 | 0.885 | 0.945 | 0.736 | 0.500 |
| 95% CI | 0.585 to 0.894 | 0.726 to 0.968 | 0.805 to 0.992 | 0.560 to 0.870 | 0.327 to 0.673 |

TABLE 13
Examples of the performance of combination of lung markers based
on ratio on sputum samples from lung cancer patients and from control
patients (cncr: cancer; ctrl: control).

| | RATIO | |
|---|---|---|
| Assays | RASSF1A/SOX17 | RASSF1A/HS3ST2_nor |
| Sputum Cncr test+ | 6 | 5 |
| Sputum Cncr test− | 2 | 3 |
| Sputum Cntrl test+ | 2 | 2 |
| Sputum Cntrl test− | 25 | 25 |
| p-value (Fisher test) | 4.27E−04 | 3.04E−04 |
| Sensitivity | 75% | 63% |
| Specificity | 93% | 93% |

TABLE 14
Examples of the performance of combination of lung markers based on copy number on sputum
samples from lung cancer patients and from control patients (cncr: cancer; ctrl: control).

| | COPY NUMBER | | | | | | | |
|---|---|---|---|---|---|---|---|---|
| | NID2/ | HS3ST2_nor/ | SOX17/ | NID2/ | SOX17/ | NLD2/ | NID2/ | RASSF1A/ |
| Assays | SOX17 | RASSF1A | HS3ST2_nor | RASSF1A | RASSF1A | HS3ST2_nor | SFRP1 | SFRP1 |
| Sputum | 8 | 8 | 8 | 7 | 7 | 7 | 6 | 5 |
| Cncr test+ | | | | | | | | |
| Sputum | 0 | 0 | 0 | 1 | 1 | 1 | 2 | 3 |
| Cncr test− | | | | | | | | |
| Sputum | 2 | 2 | 3 | 2 | 2 | 3 | 2 | 3 |
| Cntrl test+ | | | | | | | | |
| Sputum | 25 | 25 | 24 | 25 | 25 | 24 | 25 | 24 |
| Cntrl test− | | | | | | | | |
| p-value | 1.91E−06 | 1.91E−06 | 7.01E−06 | 4.02E−05 | 4.02E−05 | 1.29E−04 | 4.27E−04 | 7.39E−03 |
| (Fisher test) | | | | | | | | |
| Sensitivity | 100% | 100% | 100% | 88% | 88% | 88% | 75% | 63% |
| Specificity | 93% | 93% | 89% | 93% | 93% | 89% | 93% | 89% |

### REFERENCES

The disclosure of each reference cited in this disclosure is expressly incorporated herein.
Barringer K J, Orgel L, Wahl G, Gingeras T R. Gene. 1990 Apr. 30; 89(1):117-22
Esteller M, Corn P G, Baylin S B, Herman J G. A gene hypermethylation profile of human cancer. Cancer Res. 2001 Apr. 15; 61(8):3225-9.
Cross, S H et al. Nature Genetics 1994, 6, 236-244
Deng, D. et al. Simultaneous detection of CpG methylation and single nucleotide polymorphism by denaturing high performance liquid chromatography. 2002 Nuc. Acid Res, 30, 3.
Ganti, and Mulshine. Lung cancer screening. The Oncologist 2006, Vol. 11, No. 5, 481-487
Gentleman R C, Carey V J, Bates D M, Bolstad B, Dealing M, et al. (2004) Bioconductor: open software development for computational biology and bioinformatics. Genome Biol 5: R80.
Greenberg and Lee. Biomarkers for Lung Cancer: Clinical Uses Curr Opin Pulm Med. 2007; 13(4):249-255.
Greenlee, Hill-Harmon, Murray and Thun, Cancer statistics 2001, CA Cancer J. Clin. 2001; 51: 15-36.
Ihaka R, Gentleman R C (1996) A language for data analysis and graphics. Journal of Computational and Graphical Statistics 5: 299-314.
Kwoh D Y, Davis G R, Whitfield K M, Chappelle H L, DiMichele L J, Gingeras T R. Transcription-based amplification system and detection of amplified human immunodeficiency virus type 1 with a bead-based sandwich hybridization format. Proc Natl Acad Sci USA. 1989 February; 86(4):1173-7.
Li K B. ClustalW-MPI: ClustalW analysis using distributed and parallel computing. Bioinformatics 2003; 19(12):1585-6.
Schuebel K E, Chen W, Cope L, Glockner S C, Suzuki H, Yi J M, Chan T A, Van Neste L, Van Criekinge W, van den Bosch S, van Engeland M, Ting A H, Jair K, Yu W, Toyota M, Imai K, Ahuja N, Herman J G & Baylin S B (2007). Comparing the DNA hypermethylome with Gene Mutations in Human Colorectal Cancer. PLoS Genetics, 3 (8), Early Online Release.
Shiraisi, Met al. Biol Chem. 1999, 380(9):1127-1131
Sjöblom T, Jones S, Wood L D, Parsons D W, Lin J, Barber T D, Mandelker D, Leary R J, Ptak J, Silliman N, Szabo S, Buckhaults P, Farrell C, Meeh P, Markowitz S D, Willis J, Dawson D, Willson J K, Gazdar A F, Hartigan J, Wu L, Liu C, Parmigiani G, Park B H, Bachman K E, Papadopoulos N, Vogelstein B, Kinzler K W & Velculescu V E (2006). The Consensus Coding Sequences of Human Breast and Colorectal Cancers. Science, 314 (5797), 268-274.
Straub, J. et al., A64-AACRMD (2007): Base5, a versatile, highly integrated high-throughput methylation profiling platform for Methylation-Specific PCR based marker identification applied to CRC
Suzuki Y, Yamashita R, Sugano S, Nakai K. DBTSS, DataBase of Transcriptional Start Sites: progress report 2004. Nucleic Acids Res 2004; 32 (Database issue):D78-81.
Suzuki Y, Yamashita R, Nakai K, Sugano S. DBTSS: DataBase of human Transcriptional Start Sites and full-length cDNAs. Nucleic Acids Res 2002; 30(1):328-31.
Thompson J D, Higgins D G, Gibson T J. CLUSTAL W: improving the sensitivity of progressive multiple sequence alignment through sequence weighting, position-specific gap penalties and weight matrix choice. Nucleic Acids Res 1994; 22(22):4673-80.
Tost, J. et al. Analysis and accurate quantification of CpG methylation by MALDI mass spectrometry. Nuc. Acid Res, 2003, 31(9): e50
Travis W. D., Pathology of lung cancer, Clin. Chest Med. 23 (2002), 65-81.
Trooskens G, De Beule D, Decouttere F, Van Criekinge W. Phylogenetic trees: visualizing, customizing and detecting incongruence. Bioinformatics 2005; 21(19):3801-2.

1. A method for identifying lung cancer or its precursor, or predisposition to lung cancer, comprising:
detecting in a test sample containing lung cells or nucleic acids from lung cells, epigenetic modification of at least one gene selected from the group consisting of DPYSL4, SULF2, JAM3, APC2, BMP7, ACSL6, ALS2CL, ARTS-1, BEX1, BNIP3, CBR3, CD248, CD44, CHD5, DLK1, DSC2, EDNRB, EPB41L3, EPHB6, ERBB3, FBLN2, FBN2, FOXL2, GNAS, GSTP1, HS3ST2, HPN, IGFBP7, IRF7, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, TIMP3, TJP2, TRPV2, UCHL1, WDR69, ZFP42, ZNF442, and ZNF655; and
identifying the test sample as containing cells that are neoplastic, precursor to neoplastic, or predisposed to neoplasia, or as containing nucleic acids from cells that are neoplastic, precursor to neoplastic, or predisposed to neoplasia.

2. The method of claim 1 wherein the test sample contains squamous cells or nucleic acids from squamous cells.

3. The method of claim 1 wherein the test sample contains adenocarcinoma cells or nucleic acids from adenocarcinoma cells.

4. The method of claim 1 wherein the test sample contains large cell carcinoma cells or nucleic acids from large cell carcinoma cells.

5. The method of claim 1 wherein the test sample contains a mixture of squamous cells, adenocarcinoma cells, and large cell carcinoma cells.

6. The method of claim 1 wherein the test sample is from a specimen selected from the group consisting of a tissue specimen, a biopsy specimen, a surgical specimen, a cytological specimen, sputum specimen, pleural fluid and a bronchoalveolar lavage.

7. The method of claim 6 wherein the test sample is from a biopsy specimen and surgical removal of neoplastic tissue is recommended to the patient

8. The method of claim 6 wherein the specimen is a surgical specimen and adjuvant chemotherapy or adjuvant radiation therapy is recommended to the patient.

9. The method of claim 1 wherein an epigenetic modification in a panel of genes comprising two, three, four or five genes is detected, wherein detection of an epigenetic change in at least one of the genes in the panel is indicative of a predisposition to, or the incidence of lung cancer.

10. The method of claim 9 wherein epigenetic modification of RASSF1A and/or SOX17 and/or HS3ST2-nor and/or NID2 and/or SFRP1 is detected

11. The method of claim 1 wherein epigenetic modification is detected by detecting methylation of a CpG dinucleotide motif in the gene.

12. The method of claim 1 wherein epigenetic modification is detected by detecting methylation of a CpG dinucleotide motif in a promoter, intron or exon of the gene.

13. The method of claim 1 wherein epigenetic modification is detected by detecting diminished expression of mRNA of the gene.

14. The method of claim 11 wherein methylation is detected by contacting at least a portion of the gene with a methylation-sensitive restriction endonuclease, said endonuclease preferentially cleaving methylated recognition sites relative to non-methylated recognition sites, whereby cleavage of the portion of the gene indicates methylation of the portion of the gene.

15. The method of claim 11 wherein methylation is detected by contacting at least a portion of the gene with a methylation-sensitive restriction endonuclease, said endonuclease preferentially cleaving non-methylated recognition sites relative to methylated recognition sites, whereby cleavage of the portion of the gene indicates non-methylation of the portion of the gene provided that the gene comprises a recognition site for the methylation-sensitive restriction endonuclease.

16. The method of claim 11 wherein methylation is detected by:
contacting at least a portion of the gene of the test sample with a chemical reagent that selectively modifies a non-methylated cytosine residue relative to a methylated cytosine residue, or selectively modifies a methylated cytosine residue relative to a non-methylated cytosine residue; and
detecting a product generated due to said contacting.

17. The method of claim 16 wherein the step of detecting a product employs amplification with at least one primer that hybridizes to a sequence comprising a modified non-methylated CpG dinucleotide motif but not to a sequence comprising an unmodified methylated CpG dinucleotide motif thereby forming amplification products.

18. The method of claim 16 wherein the step of detecting a product comprises amplification with at least one primer that hybridizes to a sequence comprising an unmodified methylated CpG dinucleotide motif but not to a sequence comprising a modified non-methylated CpG dinucleotide motif thereby forming amplification products.

19. The method of claim 16 wherein the product is detected by a method selected from the group consisting of electrophoresis, hybridization, amplification, sequencing, ligase chain reaction, chromatography, mass spectrometry, and combinations thereof.

20. The method of claim 16 wherein the chemical reagent is hydrazine.

21. The method of claim 20 further comprising cleavage of the hydrazine-contacted at least a portion of the gene with piperidine.

22. The method of claim 16 wherein the chemical reagent comprises bisulfite ions.

23. The method of claim 22 further comprising treating the bisulfite ion-contacted, at least a portion of the gene with alkali.

24. The method of claim 1 wherein the step of detecting employs amplification of at least a portion of the at least one gene using an oligonucleotide primer that specifically hybridizes under amplification conditions to a region of a gene selected from the group consisting of DPYSL4, SULF2, JAM3, APC2, BMP7, ACSL6, ALS2CL, ARTS-1, BEX1, BNIP3, CBR3, CD248, CD44, CHD5, DLK1, DSC2, EDNRB, EPB41L3, EPHB6, ERBB3, FBLN2, FBN2, FOXL2, GNAS, GSTP1, HS3ST2, HPN, IGFBP7, IRF7, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, TIMP3, TJP2, TRPV2, UCHL1, WDR69, ZFP42, ZNF442, and ZNF655; wherein the region is within about 3 kb of said gene's transcription start site.

25. The method of claim 1 wherein the step of detecting employs amplification of at least a portion of the at least one gene using at least one pair of oligonucleotide primers that specifically hybridizes under amplification conditions to a region of a gene selected from the group consisting of DPYSL4, SULF2, JAM3, APC2, BMP7, ACSL6, ALS2CL, ARTS-1, BEX1, BNIP3, CBR3, CD248, CD44, CHD5, DLK1, DSC2, EDNRB, EPB41L3, EPHB6, ERBB3, FBLN2, FBN2, FOXL2, GNAS, GSTP1, HS3ST2, HPN, IGFBP7, IRF7, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, TIMP3, TJP2, TRPV2, UCHL1, WDR69, ZFP42, ZNF442, and ZNF655; wherein the region is within about 3 kb of said gene's transcription start site.

26. The method of claim 25 wherein the region comprise, consist essentially of or consist of the sequences represented by SEQ ID NO. 129-192 and/or SEQ ID NO. 193-256 and/or SEQ ID NO. 315-329 and/or SEQ ID NO. 330-344 and/or SEQ ID NO. 408-428 and/or SEQ ID NO. 429-449 and/or SEQ ID NO. 271-277 and/or SEQ ID NO. 278-284.

27. The method of claim 1 wherein the step of detecting a product comprises amplification with at least one sense primer comprising, consisting essentially of or consisting of SEQ ID NO. 1-64 and/or SEQ ID NO. 285-299 and/or SEQ ID NO. 345-365 and/or SEQ ID NO. 257-263.

28. The method of claim 1 wherein the step of detecting a product comprises amplification with at least one antisense primer comprising, consisting essentially of or consisting of SEQ ID NO. 65-128 and/or SEQ ID NO. 300-314 and/or SEQ ID NO. 366-386 and/or SEQ ID NO. 264-270.

29. The method of claim 1 wherein the step of detecting employs amplification of at least a portion of the at least one gene, and further employs at least one oligonucleotide probe which hybridizes to an amplicon selected from the group consisting of SEQ ID NO: 129-292 and/or SEQ ID NO. 193-256 and/or SEQ ID NO. 315-329 and/or SEQ ID NO. 330-344 and/or SEQ ID NO. 408-428 and/or SEQ ID NO. 429-449 and/or SEQ ID NO. 271-277 and/or SEQ ID NO. 278-284.under amplification conditions.

30. The method of claim 29 wherein the probe comprises, consists essentially of or consists of sequences represented by SEQ ID NO. 387-407.

31. The method of claim 1 wherein the step of detecting employs amplification of at least a portion of the at least one gene and a detectable reagent which preferentially binds to double stranded DNA relative to single stranded DNA.

32. The method of claim 25 wherein an oligonucleotide probe is covalently linked to the oligonucleotide primer.

33. A kit for assessing lung cancer or its precursor, or predisposition to lung cancer in a test sample containing lung cells or nucleic acids from lung cells, said kit comprising in a package:
a reagent that (a) modifies methylated cytosine residues but not non-methylated cytosine residues, or that (b) modifies non-methylated cytosine residues but not methylated cytosine residues; and
at least one pair of oligonucleotide primers that specifically hybridizes under amplification conditions to a region of a gene selected from the group consisting of DPYSL4, SULF2, JAM3, APC2, BMP7, ACSL6, ALS2CL, ARTS-1, BEX1, BNIP3, CBR3, CD248, CD44, CHD5, DLK1, DSC2, EDNRB, EPB41L3, EPHB6, ERBB3, FBLN2, FBN2, FOXL2, GNAS, GSTP1, HS3ST2, HPN, IGFBP7, IRF7, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, TIMP3, TJP2, TRPV2, UCHL1, WDR69, ZFP42, ZNF442, and ZNF655; wherein the region is within about 3 kb of said gene's transcription start site.

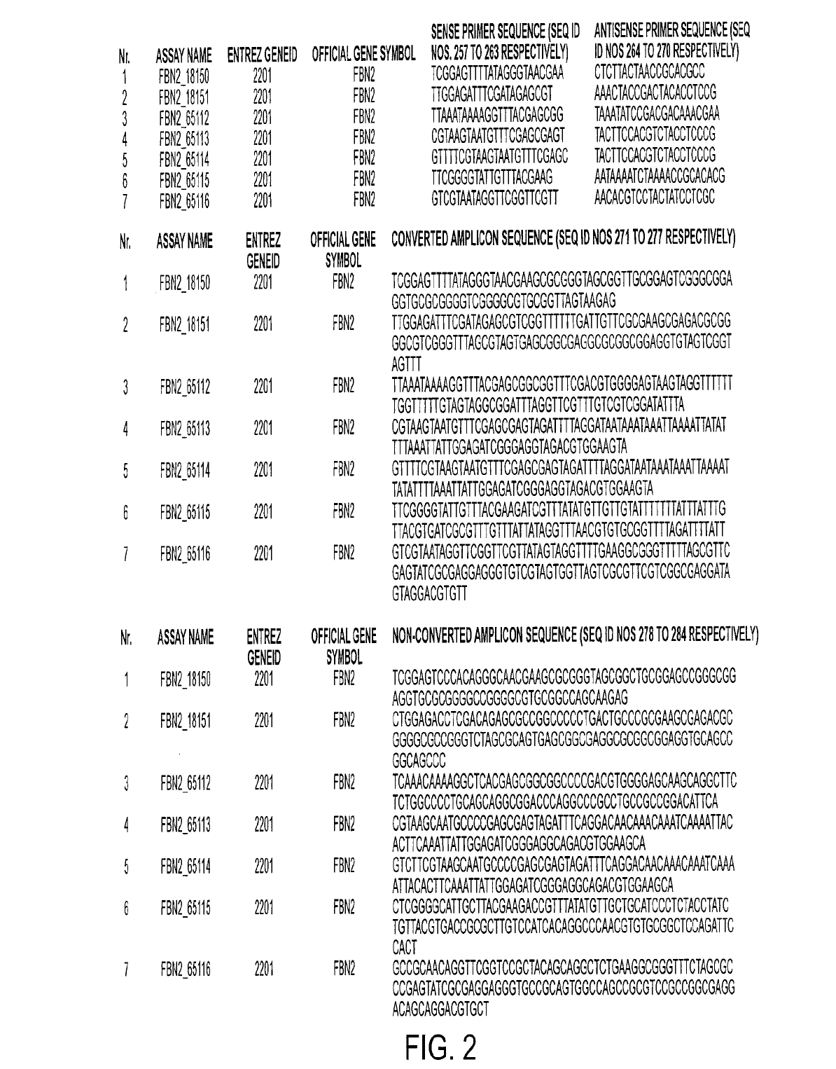

34. The kit of claim 33 wherein the at least one pair of primers is selected from Table 1 (SEQ ID NO: 1-128), FIG. 2 (SEQ ID NO: 257-270), Table 3 (SEQ ID NO: 285-314) and Table 7 (SEQ ID NO: 345-386).

35. The kit of claim 33 wherein the at least one pair of oligonucleotide primers amplifies an amplicon selected from Table 2 (SEQ ID NO: 129-256), FIG. 2 (SEQ ID NO: 271-284), Table 4 (SEQ ID NO: 315-344) and Table 8 (SEQ ID NO:408-449).

36. A kit for assessing lung cancer or its precursor, or predisposition to lung cancer in a test sample containing lung cells or nucleic acids from lung cells, said kit comprising in a package:
at least two pairs of oligonucleotide primers that specifically hybridize under amplification conditions to a region of a gene selected from the group consisting of DPYSL4, SULF2, JAM3, APC2, BMP7, ACSL6, ALS2CL, ARTS-1, BEX1, BNIP3, CBR3, CD248, CD44, CHD5, DLK1, DSC2, EDNRB, EPB41L3, EPHB6, ERBB3, FBLN2, FBN2, FOXL2, GNAS, GSTP1, HS3ST2, HPN, IGFBP7, IRF7, LOX, LY6D, LY6K, MACF1, MCAM, NCBP1, NEFH, NID2, PCDHB15, PCDHGA12, PFKP, PGRMC1, PHACTR3, PHKA2, POMC, PRKCA, PSEN1, RASSF1A, RASSF2, RBP1, RRAD, SFRP1, SGK, SOD3, SOX17, TIMP3, TJP2, TRPV2, UCHL1, WDR69, ZFP42, ZNF442, and ZNF655; wherein the region is within about 3 kb of said gene's transcription start site.

37. The kit of claim 36 wherein the at least two pairs of primers are selected from SEQ ID NO: 1-128 (Table 1), SEQ ID NO: 257-270 (FIG. 2), SEQ ID NO:285-314 (Table 3), SEQ ID NO: 345-386 (Table 7).

38. The kit of claim 36 wherein the at least two pairs of oligonucleotide primers amplify amplicons selected from Table 2 (SEQ ID NO: 129-256), FIG. 2 (SEQ ID NO: 271-284), Table 4 (SEQ ID NO: 315-344) and Table 8 (SEQ ID NO: 408-449).

39. The kit of claim 33 or 36 further comprising at least one oligonucleotide probe which hybridizes to an amplicon selected from the group consisting of Table 2 (SEQ ID NO: 129-256), FIG. 2 (SEQ ID NO: 271-284), Table 4 (SEQ ID NO: 315-344), Table 8 (SEQ ID NO: 408-449) under amplification conditions.

40. The kit of claim 39 wherein the oligonucleotide probe is selected from the group consisting of SEQ ID NO: 387-407.

41. The kit of claim 40 wherein the oligonucleotide probe comprises a fluorescent label.

42. The kit of claim 40 wherein the oligonucleotide probe comprises a fluorescence quenching agent.

43. The kit of claim 40 wherein the oligonucleotide probe comprises a fluorescent label and fluorescence quenching agent.

44. The kit of claim 33 or 36 which comprises a detectable reagent which preferentially binds to double stranded DNA relative to single stranded DNA.

45. The kit of claim 33 or 36 further comprising a DNA polymerase for amplifying DNA.

46. The kit of claim 33 or 36 further comprising at least one oligonucleotide probe which is covalently linked to at least one of said oligonucleotide primers.

47. An isolated polynucleotide comprising a nucleotide sequence selected from the group consisting of SEQ ID NO: 1-449.

48. The polynucleotide of claim 41 which is detectably labeled.

49. The polynucleotide of claim 41 which is detectably labeled with a fluorescent label.

50. The isolated polynucleotide of claim 41 which consists of the selected nucleotide sequence.

51. The method of claim 1 wherein epigenetic modification is detected by detecting hypomethylation of a CpG dinucleotide motif in the gene.

52. The method of claim 1 wherein epigenetic modification is detected by detecting hypomethylation of a CpG dinucleotide motif in a promoter of the gene.

53. The method of claim 1 wherein epigenetic modification is detected by detecting increased expression of mRNA of the gene.


SEQUENCE LISTING (449 sequences)

SEQ ID NO: 1 - DNA, 22 bp, Homo sapiens
tttaatgttacgttttggcgtt

SEQ ID NO: 2 - DNA, 21 bp, Homo sapiens
gcggttgtaaggtttttggtc

SEQ ID NO: 3 - DNA, 21 bp, Homo sapiens
ggacgggtgtttgcgttttac

SEQ ID NO: 4 - DNA, 22 bp, Homo sapiens
gtcgtttgtttaggttcggatc

SEQ ID NO: 5 - DNA, 20 bp, Homo sapiens
tcggggtttttatttggttc

SEQ ID NO: 6 - DNA, 20 bp, Homo sapiens
gtacgtgcgtttattgcgag

SEQ ID NO: 7 - DNA, 20 bp, Homo sapiens
ggtatcggtttggttatcgc

SEQ ID NO: 8 - DNA, 25 bp, Homo sapiens
gttttcgattgatttattaaggttc

SEQ ID NO: 9 - DNA, 19 bp, Homo sapiens
tcgtgggaagagagcgtag

SEQ ID NO: 10 - DNA, 25 bp, Homo sapiens
ttttgttaagagttgtcgttagttc

SEQ ID NO: 11 - DNA, 20 bp, Homo sapiens
ggggtagtcgttaattgcgt

SEQ ID NO: 12 - DNA, 22 bp, Homo sapiens
cgggagaagaaagttagtgcgt

SEQ ID NO: 13 - DNA, 17 bp, Homo sapiens
gagcgttcgggttttgc

SEQ ID NO: 14 - DNA, 25 bp, Homo sapiens
ttgggaatttagttgtcgtcgtttc

SEQ ID NO: 15 - DNA, 20 bp, Homo sapiens
gaggtttgcggtttaggttc

SEQ ID NO: 16 - DNA, 20 bp, Homo sapiens
ggagttggggtttacgagac

SEQ ID NO: 17 - DNA, 25 bp, Homo sapiens
ggtgttttgatagaagtcgttagtc

SEQ ID NO: 18 - DNA, 20 bp, Homo sapiens
ggggttatagtttggcgttc

SEQ ID NO: 19 - DNA, 21 bp, Homo sapiens
ggtttcggtttcgttttgttc

SEQ ID NO: 20 - DNA, 19 bp, Homo sapiens
gggatagtggggttgacgc

SEQ ID NO: 21 - DNA, 19 bp, Homo sapiens
gcgtgggttttcgtcgtag

SEQ ID NO: 22 - DNA, 24 bp, Homo sapiens
gggtgttcgatttaagtcgagttc

SEQ ID NO: 23 - DNA, 22 bp, Homo sapiens
gtttagttaagttcggttcggg

SEQ ID NO: 24 - DNA, 24 bp, Homo sapiens
agggagtttagttaagttcggttc

SEQ ID NO: 25 - DNA, 19 bp, Homo sapiens
tagagcggaggaagttgcg

SEQ ID NO: 26 - DNA, 23 bp, Homo sapiens
tcggagttttatagggtaacgaa

SEQ ID NO: 27 - DNA, 21 bp, Homo sapiens
ttggagatttcgatagagcgt

SEQ ID NO: 28 - DNA, 26 bp, Homo sapiens
gcgataggtttttagtaagtaagcgc

SEQ ID NO: 29 - DNA, 20 bp, Homo sapiens
ttcggggtgtagcggtcgtc

SEQ ID NO: 30 - DNA, 20 bp, Homo sapiens
acgtaagagtttgggagcgt

SEQ ID NO: 31 - DNA, 19 bp, Homo sapiens
gtttcggggttcgtttttc

SEQ ID NO: 32 - DNA, 21 bp, Homo sapiens
tttgtcggcgtcgttattttc

SEQ ID NO: 33 - DNA, 21 bp, Homo sapiens
cgtttatgggtcggttacgtc

SEQ ID NO: 34 - DNA, 19 bp, Homo sapiens
agttgagaatcggacgggg

SEQ ID NO: 35 - DNA, 20 bp, Homo sapiens
gggattataagtcgcgtcgc

SEQ ID NO: 36 - DNA, 22 bp, Homo sapiens
gcgcgtagagttgtaaaggttc

SEQ ID NO: 37 - DNA, 21 bp, Homo sapiens
ggtagaggcgaggagttgttc

SEQ ID NO: 38 - DNA, 21 bp, Homo sapiens
gatgtcgtttgggagtagtgc

SEQ ID NO: 39 - DNA, 27 bp, Homo sapiens
gcggggttttttttatcggttagattc

SEQ ID NO: 40 - DNA, 23 bp, Homo sapiens
gttttcgttgtcgttacgggttc

SEQ ID NO: 41 - DNA, 23 bp, Homo sapiens
agaatttaggtcggtttttatcg

SEQ ID NO: 42 - DNA, 19 bp, Homo sapiens
gtcggatgaagtattcggg

SEQ ID NO: 43 - DNA, 24 bp, Homo sapiens
ttatttcgtttttagggagttttc

SEQ ID NO: 44 - DNA, 21 bp, Homo sapiens
tttcgtgtgggaagagttcgt

SEQ ID NO: 45 - DNA, 25 bp, Homo sapiens
ttttggttattaggtagttcggttc

SEQ ID NO: 46 - DNA, 19 bp, Homo sapiens
ttattttgcgagcggtttc

SEQ ID NO: 47 - DNA, 25 bp, Homo sapiens
gatttgggcgtttttggtttttcgc

SEQ ID NO: 48 - DNA, 20 bp, Homo sapiens
gggcgttgaggtagaagaac

SEQ ID NO: 49 - DNA, 20 bp, Homo sapiens
ttaggtcggaggtttcgttt

SEQ ID NO: 50 - DNA, 20 bp, Homo sapiens
gatgtttcggtcgaggtttc

SEQ ID NO: 51 - DNA, 26 bp, Homo sapiens
tgtagttttcggagttagtgtcgcgc

SEQ ID NO: 52 - DNA, 21 bp, Homo sapiens
ttttgttcgtcgtattttcgg

SEQ ID NO: 53 - DNA, 22 bp, Homo sapiens
agtatagagtggggagcgtagc

SEQ ID NO: 54 - DNA, 21 bp, Homo sapiens
ttgcgttagtcgtttgcgttc

SEQ ID NO: 55 - DNA, 22 bp, Homo sapiens
gttagtcgagttcggaggtatc

SEQ ID NO: 56 - DNA, 21 bp, Homo sapiens
gcgtcggaggttaaggttgtt

SEQ ID NO: 57 - DNA, 21 bp, Homo sapiens
cgggttagagtattgttcggt

SEQ ID NO: 58 - DNA, 21 bp, Homo sapiens
gattttatcggggaaatatcg

SEQ ID NO: 59 - DNA, 25 bp, Homo sapiens
ttatttcgtaggttgaggttagggc

SEQ ID NO: 60 - DNA, 22 bp, Homo sapiens
gttgtattttcgcggagcgttc

SEQ ID NO: 61 - DNA, 24 bp, Homo sapiens
gtttaggttgtggtttaggtcgtc

SEQ ID NO: 62 - DNA, 26 bp, Homo sapiens
ggggtttttaggtattcggttcgtac

SEQ ID NO: 63 - DNA, 24 bp, Homo sapiens
tcggtttttagttttttcggtcgc

SEQ ID NO: 64 - DNA, 21 bp, Homo sapiens
ttatcgagaagcgtcggtttc

SEQ ID NO: 65 - DNA, 19 bp, Homo sapiens
gaaccaaccctctccgacc

SEQ ID NO: 66 - DNA, 19 bp, Homo sapiens
atttttccgcaacctctcg

SEQ ID NO: 67 - DNA, 27 bp, Homo sapiens
cgaaaccaaaaaactaaacgaaaaccg

SEQ ID NO: 68 - DNA, 22 bp, Homo sapiens
gacccgaaataacctcgaaacg

SEQ ID NO: 69 - DNA, 21 bp, Homo sapiens
aatcgtcactcgtatctcgct

SEQ ID NO: 70 - DNA, 23 bp, Homo sapiens
cgttatccaaactaaaatcgacc

SEQ ID NO: 71 - DNA, 22 bp, Homo sapiens
cgcctacaactactacacgacc

SEQ ID NO: 72 - DNA, 22 bp, Homo sapiens
tcaaaatccgaactctaaaccg

SEQ ID NO: 73 - DNA, 22 bp, Homo sapiens
ttactaacctaaacgaccgcaa

SEQ ID NO: 74 - DNA, 21 bp, Homo sapiens
aatataaaccctacgaccgcc

SEQ ID NO: 75 - DNA, 19 bp, Homo sapiens
tcttccccgaaaaccgcta

SEQ ID NO: 76 - DNA, 24 bp, Homo sapiens
aaatcgaaaaacctaaaatatcgc

SEQ ID NO: 77 - DNA, 22 bp, Homo sapiens
cgacctcgacgaaaaaataacg

SEQ ID NO: 78 - DNA, 25 bp, Homo sapiens
aaacaacgactaccgatactacgcg

SEQ ID NO: 79 - DNA, 22 bp, Homo sapiens
ctcacactatacaacacgcgac

SEQ ID NO: 80 - DNA, 21 bp, Homo sapiens
ataataaattccccgacgacc

SEQ ID NO: 81 - DNA, 19 bp, Homo sapiens
aaaaccattaacgcccacg

SEQ ID NO: 82 - DNA, 21 bp, Homo sapiens
gctctaaaaaccacacccgtc

SEQ ID NO: 83 - DNA, 21 bp, Homo sapiens
ctctacgactcaaacctcgct

SEQ ID NO: 84 - DNA, 20 bp, Homo sapiens
ataaaaatcccgacgaacga

SEQ ID NO: 85 - DNA, 19 bp, Homo sapiens
cccaaaactactcgccgct

SEQ ID NO: 86 - DNA, 24 bp, Homo sapiens
cgcgaatcttaaccgaaaaaatcg

SEQ ID NO: 87 - DNA, 23 bp, Homo sapiens
gattacaatttacaacctccgct

SEQ ID NO: 88 - DNA, 19 bp, Homo sapiens
tacaacctccgctaccgtc

SEQ ID NO: 89 - DNA, 22 bp, Homo sapiens
caaatacgaacacaaaaaccga

SEQ ID NO: 90 - DNA, 19 bp, Homo sapiens
ctcttactaaccgcacgcc

SEQ ID NO: 91 - DNA, 21 bp, Homo sapiens
aaactaccgactacacctccg

SEQ ID NO: 92 - DNA, 24 bp, Homo sapiens
ctctccgctccaaacgctaacgcg

SEQ ID NO: 93 - DNA, 22 bp, Homo sapiens
gccccaatactaaatcacgacg

SEQ ID NO: 94 - DNA, 21 bp, Homo sapiens
gactcctcgaaaaacaaacga

SEQ ID NO: 95 - DNA, 20 bp, Homo sapiens
cgactcgctctatctcgcac

SEQ ID NO: 96 - DNA, 24 bp, Homo sapiens
aaactacctactaaacgaaacccg

SEQ ID NO: 97 - DNA, 21 bp, Homo sapiens
ataaaaacacgaaaaccccgc

SEQ ID NO: 98 - DNA, 20 bp, Homo sapiens
aacgaatcaaactcccgaaa

SEQ ID NO: 99 - DNA, 20 bp, Homo sapiens
cgaacgcaaaaccgaaatcg

SEQ ID NO: 100 - DNA, 19 bp, Homo sapiens
acgtcctcctcgaacgaaa

SEQ ID NO: 101 - DNA, 22 bp, Homo sapiens
tacacaaaccgttctaacccga

SEQ ID NO: 102 - DNA, 23 bp, Homo sapiens
acaaaataccgctaactaacgaa

SEQ ID NO: 103 - DNA, 27 bp, Homo sapiens
caacgatacccaaaaaaaatcaacgcg

SEQ ID NO: 104 - DNA, 17 bp, Homo sapiens
gcgcaacgaacaaaacg

SEQ ID NO: 105 - DNA, 22 bp, Homo sapiens
acgcaaaattcttctcccaaaa

SEQ ID NO: 106 - DNA, 20 bp, Homo sapiens
ccctacaaacgacgacgaac

SEQ ID NO: 107 - DNA, 21 bp, Homo sapiens
cttacgaaccatttaatcccg

SEQ ID NO: 108 - DNA, 21 bp, Homo sapiens
cgaataaccgaacgaccgata

SEQ ID NO: 109 - DNA, 22 bp, Homo sapiens
cactcttcgtactattcccgct

SEQ ID NO: 110 - DNA, 23 bp, Homo sapiens
gaatactctaattccacgcgact

SEQ ID NO: 111 - DNA, 20 bp, Homo sapiens
gacttctcataccgcaatcg

SEQ ID NO: 112 - DNA, 22 bp, Homo sapiens
cgacacctaccaaataaaatcg

SEQ ID NO: 113 - DNA, 20 bp, Homo sapiens
aaaccctcaccgttatcgtc

SEQ ID NO: 114 - DNA, 24 bp, Homo sapiens
aaacgactacaaataaatacgcca

SEQ ID NO: 115 - DNA, 25 bp, Homo sapiens
cctacgatcgaaaacgacgcgaacg

SEQ ID NO: 116 - DNA, 22 bp, Homo sapiens
ataacgaccctcgacctacgat

SEQ ID NO: 117 - DNA, 20 bp, Homo sapiens
ctttcctaccaccgaaacga

SEQ ID NO: 118 - DNA, 24 bp, Homo sapiens
caaaaacgaatcccgtatccgacg

SEQ ID NO: 119 - DNA, 24 bp, Homo sapiens
caactccgaacgaaacaataaacg

SEQ ID NO: 120 - DNA, 22 bp, Homo sapiens
ctctccaaaattaccgtacgcg

SEQ ID NO: 121 - DNA, 19 bp, Homo sapiens
gaacacaaatcccgcgtaa

SEQ ID NO: 122 - DNA, 19 bp, Homo sapiens
aaacaaatcccgctccgaa

SEQ ID NO: 123 - DNA, 21 bp, Homo sapiens
tcctctactatcaacgccgac

SEQ ID NO: 124 - DNA, 22 bp, Homo sapiens
ctcacaatacgtctaaccgacg

SEQ ID NO: 125 - DNA, 25 bp, Homo sapiens
acacctcgtatcctcactaaaaacg

SEQ ID NO: 126 - DNA, 22 bp, Homo sapiens
aatacgcaatacccgacgaccg

SEQ ID NO: 127 - DNA, 29 bp, Homo sapiens
caattactacgcaaaaacgaaacaaaacg

SEQ ID NO: 128 - DNA, 26 bp, Homo sapiens
accgaaaaaaaaaacgaacctaaccg

SEQ ID NO: 129 - DNA, 106 bp, Homo sapiens
tttaatgttacgttttggcgttcgtcgttcgtgtttttttttttagtcggttttcgtagaatgttaggtattgacgttggagagcggggtcggagagggttggttc

SEQ ID NO: 130 - DNA, 101 bp, Homo sapiens
gcggttgtaaggtttttggtcggtgagtgaattagtaggtaaggatggtagttagggtatttatatttacgagggtggtggtcgagaggttgcggaaaaat

SEQ ID NO: 131 - DNA, 91 bp, Homo sapiens
ggacgggtgtttgcgttttacgtttagttcgtttaggtgggggttttcgttttttcggttgttgcggttttcgtttagttttttggtttcg

SEQ ID NO: 132 - DNA, 99 bp, Homo sapiens
gtcgtttgtttaggttcggatcgggttttgttcgtttcggagtttttgttcgcgtcgcggagatttcggagttcgcgcgtttcgaggttatttcgggtc

SEQ ID NO: 133 - DNA, 96 bp, Homo sapiens
tcggggtttttatttggttcgttttttttcgggtcggatgttagttcgtcgagcgtagggtagcggggagttggtagcgagatacgagtgacgatt

SEQ ID NO: 134 - DNA, 104 bp, Homo sapiens
gtacgtgcgtttattgcgagttgcggcgtcgtatagtttcgtggcgttttgggtatttttgtttttgttgcgtttcgttttggtcgattttagtttggataacg

SEQ ID NO: 135 - DNA, 111 bp, Homo sapiens
ggtatcggtttggttatcgcgcgcgaattgtgtcgatagttttttggggatgtggtgtttatcgcgcgggacgtggcgcggggttaggcggtcgtgtagtagttgtaggcg

SEQ ID NO: 136 - DNA, 80 bp, Homo sapiens
gttttcgattgatttattaaggttcgatttggtttcggatatttcgtagattatttcgcggtttagagttcggattttga

SEQ ID NO: 137 - DNA, 105 bp, Homo sapiens
tcgtgggaagagagcgtagtagttgttggggtcgtaggcggtacggggtttagtagtttaggggttttggtttagtgtgggttttgcggtcgtttaggttagtaa

SEQ ID NO: 138 - DNA, 100 bp, Homo sapiens
ttttgttaagagttgtcgttagttcggggtcggattagttcgggggtatcgcgatgttgttgcgtttgttgttggtttgggcggtcgtagggtttatatt

SEQ ID NO: 139 - DNA, 95 bp, Homo sapiens
ggggtagtcgttaattgcgtttttttttttttttcgtttttaattttagagttttttattttattgttttttgttttagcggttttcggggaaga

SEQ ID NO: 140 - DNA, 90 bp, Homo sapiens
cgggagaagaaagttagtgcgtttttgggcgtaggggttagtggggttcggaggtataggtatttcgcgatattttaggtttttcgattt

SEQ ID NO: 141 - DNA, 120 bp, Homo sapiens
gagcgttcgggttttgcggggagtaggttaaggcggtcgagagaaaggggggtcgagacgggggggtggaggtttgggggggtgggggggtaggcggtcgttattttttcgtcgaggtcg

SEQ ID NO: 142 - DNA, 100 bp, Homo sapiens
ttgggaatttagttgtcgtcgtttcgtagagttttttgttttcggagggcgtttattttcgggtcgtttattattcgcgtagtatcggtagtcgttgttt

SEQ ID NO: 143 - DNA, 85 bp, Homo sapiens
gaggtttgcggtttaggttcgatttttgcgatttgttttaggtaggtttgtatgtgcgcggcggtcgcgtgttgtatagtgtgag

SEQ ID NO: 144 - DNA, 106 bp, Homo sapiens
ggagttggggtttacgagacggggcgtgcggggtatcgggcggtcggcggggagtcgtaggtttttttagagggggcgcgagtcgggtcgtcggggaatttattat

SEQ ID NO: 145 - DNA, 114 bp, Homo sapiens
ggtgttttgatagaagtcgttagtcggtgttatgtttaggataggtatttgtagttttgtgtggacgttaacgttattaggaaggattattaggtcgtgggcgttaatggtttt

SEQ ID NO: 146 - DNA, 97 bp, Homo sapiens
ggggttatagtttggcgttcggattttggttcgggttatttgcgaaggagtcggttttggttaaggtgtttttttggacgggtgtggtttttagagc

SEQ ID NO: 147 - DNA, 89 bp, Homo sapiens
ggtttcggtttcgttttgttcgttgttttcggcgacggtcgtggtttttgttttggggttaattatagagcgaggtttgagtcgtagag

SEQ ID NO: 148 - DNA, 113 bp, Homo sapiens
gggatagtggggttgacgcgtggtttcggcgtcgcgcggtttttcgaatttcgagtttcgcgttcggcgcggtcggggtttttaatcgttttttcgttcgtcgggatttttat

SEQ ID NO: 149 - DNA, 83 bp, Homo sapiens
gcgtgggttttcgtcgtagtttcgcggagtttcggtgttttttgtaatagggggcggggggaatagcggcgagtagttttggg

SEQ ID NO: 150 - DNA, 120 bp, Homo sapiens
gggtgttcgatttaagtcgagttcgagttcgagtttaggtaggagttttatagatagttttttttttttttattttttgtaggcgttttacgcgtgcgattttttcggttaagattcgcg

SEQ ID NO: 151 - DNA, 118 bp, Homo sapiens
gtttagttaagttcggttcgggggtttttaggttaggatatcgaggtaagagttatttgaatcgttggcgaattggtggttgttgcggcgacggtagcggaggttgtaaattgtaatc

SEQ ID NO: 152 - DNA, 113 bp, Homo sapiens
agggagtttagttaagttcggttcgggggtttttaggttaggatatcgaggtaagagttatttgaatcgttggcgaattggtggttgttgcggcgacggtagcggaggttgta

SEQ ID NO: 153 - DNA, 90 bp, Homo sapiens
tagagcggaggaagttgcggatttggggtgggggaattcgttcgcggatttttggtttttatttcgcgtcggtttttgtgttcgtatttg

SEQ ID NO: 154 - DNA, 87 bp, Homo sapiens
tcggagttttatagggtaacgaagcgcgggtagcggttgcggagtcgggcggaggtgcgcggggtcggggcgtgcggttagtaagag

SEQ ID NO: 155 - DNA, 111 bp, Homo sapiens
ttggagatttcgatagagcgtcggttttttgattgttcgcgaagcgagacgcggggcgtcgggtttagcgtagtgagcggcgaggcgcggcggaggtgtagtcggtagttt

SEQ ID NO: 156 - DNA, 120 bp, Homo sapiens
gcgataggtttttagtaagtaagcgcgggcggtattcgtagtttttagaagtttgagatttggtcgtaagcggattcgtgcgttttaattttttgtcgcgttagcgtttggagcggagag

SEQ ID NO: 157 - DNA, 91 bp, Homo sapiens
ttcggggtgtagcggtcgtcggggttggggtcggcgggagttcgcgggattttttagaagagcggtcggcgtcgtgatttagtattggggc

SEQ ID NO: 158 - DNA, 101 bp, Homo sapiens
acgtaagagtttgggagcgttcgagtcgttcggttgttcggagttttatcgtttaggatcgggagatgttggaaatgtaatcgtttgtttttcgaggagtc

SEQ ID NO: 159 - DNA, 96 bp, Homo sapiens
gtttcggggttcgtttttcggtaggttcggggagaggtggggtgataatgggttggggtgcgcgcgtgttttataggtgcgagatagagcgagtcg

SEQ ID NO: 160 - DNA, 97 bp, Homo sapiens
tttgtcggcgtcgttattttcgtacggttcgttttcgtcgcgggcgtatatagggtagtagtcgtacgcgtcgcgggtttcgtttagtaggtagttt

SEQ ID NO: 161 - DNA, 114 bp, Homo sapiens
cgtttatgggtcggttacgtcgggtgttcgtttatttttcgacgttagtaggagcgcgcgcgtaggtttcgcggggtcgggagggcggtacgggcggggttttcgtgtttttat

SEQ ID NO: 162 - DNA, 83 bp, Homo sapiens
agttgagaatcggacggggtgggatcgaggagggtgcgaagcgttattgtttaggtttcgttttttcgggagtttgattcgtt

SEQ ID NO: 163 - DNA, 91 bp, Homo sapiens
gggattataagtcgcgtcgcgttgtcgttggttttttagtaattttcgatatggcgttgaggcggttatcgcgatttcggttttgcgttcg

SEQ ID NO: 164 - DNA, 91 bp, Homo sapiens
gcgcgtagagttgtaaaggttcgagtaggagtacggtttaggcgaagcgtattattttttttgttagattgatttcgttcgaggaggacgt

SEQ ID NO: 165 - DNA, 92 bp, Homo sapiens
ggtagaggcgaggagttgttcgttttgtacgtttttaatcgtattacgtgaataaatagttgaggggcggtcgggttagaacggtttgtgta

SEQ ID NO: 166 - DNA, 97 bp, Homo sapiens
gatgtcgtttgggagtagtgcgggtttttgtattgttaaggttttataggtacgggttgggcgggggtgggtagttcgttagttagcggtattttgt

SEQ ID NO: 167 - DNA, 109 bp, Homo sapiens
gcggggttttttttatcggttagattcggggagaggcgcgcggaggttgcgaaggttttagaagggcggggagggggcgtcgcgcgttgattttttttgggtatcgttg

SEQ ID NO: 168 - DNA, 97 bp, Homo sapiens
gttttcgttgtcgttacgggttcgttttttttttttttcggtttttagggtaaggcgcggggcgcggggttggatgtaggcgttttgttcgttgcgc

SEQ ID NO: 169 - DNA, 113 bp, Homo sapiens
agaatttaggtcggtttttatcgttttttagaacgattgtattattgtcgttgtcgtcggtttgatattgttttagttttagtgttggtagttttgggagaagaattttgcgt

SEQ ID NO: 170 - DNA, 82 bp, Homo sapiens
gtcggatgaagtattcgggcgtttttattgcggaagggcggggatggttgtgacgtaggcgtgttcgtcgtcgtttgtaggg

SEQ ID NO: 171 - DNA, 96 bp, Homo sapiens
ttatttcgtttttagggagttttcgggttatttttttattcgggttgtttcgcggtttttaaggagttttattttcgggattaaatggttcgtaag

SEQ ID NO: 172 - DNA, 109 bp, Homo sapiens
tttcgtgtgggaagagttcgtttgggtgtagcgtcgcggttcgtaatattagtaacggtagtagtagtagtattggtaacgacgatagtatcggtcgttcggttattcg

SEQ ID NO: 173 - DNA, 97 bp, Homo sapiens
ttttggttattaggtagttcggttcggcggttcgttcggggtattagttcggtgtagggcgcggagtcgttttgtagcgggaatagtacgaagagtg

SEQ ID NO: 174 - DNA, 114 bp, Homo sapiens
ttattttgcgagcggtttcgcgatacgaggtagtcgttttcgtttttcgacgcggttatgggttcggtcggcgcgggggtaagttagagcgagtcgcgtggaattagagtattc

SEQ ID NO: 175 - DNA, 75 bp, Homo sapiens
gatttgggcgtttttggtttttcgcggtttcgagttttcgataaattttttgcgtcgattgcggtatgagaagtc

SEQ ID NO: 176 - DNA, 113 bp, Homo sapiens
gggcgttgaggtagaagaacgtgtacgaggtgaaggattataaatttatcgcgcgtttttttaagtagtttattttttgtagttattgtatcgattttatttggtaggtgtcg

SEQ ID NO: 177 - DNA, 102 bp, Homo sapiens
ttaggtcggaggtttcgtttttttttttttggttttttttttttttcgtgggtcggtcgttaacgacgttagagtcggaaatgacgataacggtgagggttt

SEQ ID NO: 178 - DNA, 103 bp, Homo sapiens
gatgtttcggtcgaggtttcgtcgtagtttttttttagtttttaggtcgcggcgtttttattcgggattttttcggatttggcgtatttatttgtagtcgttt

SEQ ID NO: 179 - DNA, 126 bp, Homo sapiens
tgtagttttcggagttagtgtcgcgcgttcgtcgtttcgcgtttttttgttcgtcgtattttcgggagtcggggcgtatttagttcgtagcgtcgttttttcgttcgcgtcgttttcgatcgtagg

SEQ ID NO: 180 - DNA, 96 bp, Homo sapiens
ttttgttcgtcgtattttcgggagtcggggcgtatttagttcgtagcgtcgttttttcgttcgcgtcgttttcgatcgtaggtcgagggtcgttat

SEQ ID NO: 181 - DNA, 80 bp, Homo sapiens
agtatagagtggggagcgtagcgacgaagaatgaatagggtttcgtgaggttttaaatattcgtttcggtggtaggaaag

SEQ ID NO: 182 - DNA, 79 bp, Homo sapiens
ttgcgttagtcgtttgcgttcgtttttagtttatattatgaaagcgtttatcggtcgtcggatacgggattcgtttttg

SEQ ID NO: 183 - DNA, 79 bp, Homo sapiens
gttagtcgagttcggaggtatcgggaggtcgagagtcgtcgggattttagttttgcgtttattgtttcgttcggagttg

SEQ ID NO: 184 - DNA, 95 bp, Homo sapiens
gcgtcggaggttaaggttgtttcgtacggttcggcgggcgagcgagttcgggttgtagtagtttcgtcggcggcgcgtacggtaattttggagag

SEQ ID NO: 185 - DNA, 99 bp, Homo sapiens
cgggttagagtattgttcggtggtgtttaggaggagtaggagtaggagtagaagtagaagcggggttcggagttgcgcgtttacgcgggatttgtgttc

SEQ ID NO: 186 - DNA, 114 bp, Homo sapiens
gattttatcggggaaatatcgcggatagtcgggttagtagcgttcggagtttattttaggtttttaaatttgtagtattttttagagcgcgcgcgttcggagcgggatttgttt

SEQ ID NO: 187 - DNA, 117 bp, Homo sapiens
ttatttcgtaggttgaggttagggcgtggcggttgttgggatttcggagttttttagtagtaggggttgcgggaggaagtgaagtcgggaggggttgtcggcgttgatagtagagga

SEQ ID NO: 188 - DNA, 108 bp, Homo sapiens
gttgtattttcgcggagcgttcggtagaaatagtttagggaagacgaaaaatagttagcggagtcgtttaggttgtagttataaagcgtcggttagacgtattgtgag

SEQ ID NO: 189 - DNA, 80 bp, Homo sapiens
gtttaggttgtggtttaggtcgtcggttttcggttatgtttagtttttttgaggtcgtttttagtgaggatacgaggtgt

SEQ ID NO: 190 - DNA, 97 bp, Homo sapiens
ggggtttttaggtattcggttcgtacgtaaatttttagttcggggttttttgattttcgcgtttattttttagttcggtcgtcgggtattgcgtatt

SEQ ID NO: 191 - DNA, 95 bp, Homo sapiens
tcggtttttagttttttcggtcgcggggtgggagttgggggttgggtcggtagtcgggatttcgggcgttttgtttcgtttttgcgtagtaattg

SEQ ID NO: 192 - DNA, 116 bp, Homo sapiens
ttatcgagaagcgtcggtttcggggttgtttatagcggttcgggagaggttgtggtggtttcgagcgcgagtgtgtaggtgataggatagcggttaggttcgttttttttttcggt

SEQ ID NO: 193 - DNA, 106 bp, Homo sapiens
ctcaatgtcacgctctggcgctcgtcgcccgtgctcccccttccagccggtttccgcagaatgccaggtactgacgttggagagcggggccggagagggctggttc

SEQ ID NO: 194 - DNA, 101 bp, Homo sapiens
gcggctgcaaggcctttggccggtgagtgaaccagtaggcaaggatggcagccagggcacccatactcacgagggtggtggccgagaggctgcggaaaaac

SEQ ID NO: 195 - DNA, 91 bp, Homo sapiens
ggacgggtgtctgcgctccacgcttagctcgtccaggtgggggctcccgcctcctcggctgctgcggtccccgcccagctccttggtcccg

SEQ ID NO: 196 - DNA, 99 bp, Homo sapiens
gccgcctgcccaggcccggaccgggctttgtccgccccggagcccctgcccgcgccgcggagaccccggagcccgcgcgctccgaggccaccccgggcc

SEQ ID NO: 197 - DNA, 96 bp, Homo sapiens
ccggggcccttacctggtccgctttcccccgggccggatgccagcccgccgagcgcagggcagcggggagctggtagcgagacacgagtgacgact

SEQ ID NO: 198 - DNA, 104 bp, Homo sapiens
gcacgtgcgctcactgcgagctgcggcgccgcacagcttcgtggcgctctgggcacccctgttcctgctgcgctccgccctggccgacttcagcctggacaacg

SEQ ID NO: 199 - DNA, 111 bp, Homo sapiens
ggcatcggcttggccatcgcgcgcgaactgtgccgacagttctctggggatgtggtgctcaccgcgcgggacgtggcgcggggccaggcggccgtgcagcagctgcaggcg

SEQ ID NO: 200 - DNA, 80 bp, Homo sapiens
gcccccgactgacccatcaaggtccgatttggcttcggacacctcgcagatcaccccgcggctcagagcccggatcctga

SEQ ID NO: 201 - DNA, 105 bp, Homo sapiens
ccgtgggaagagagcgtagcagctgctggggccgcaggcggcacggggctcagcagcccaggggtcctggcccagtgtgggccctgcggccgcccaggccagcaa

SEQ ID NO: 202 - DNA, 100 bp, Homo sapiens
ccctgtcaagagctgccgccagcccggggccggaccagtccgggggcatcgcgatgctgctgcgcctgttgctggcctgggcggccgcagggcccacact

SEQ ID NO: 203 - DNA, 95 bp, Homo sapiens
ggggcagccgtcaactgcgccttctcccctcctccgcccccaaccttagagccccccaccccactgcttcctgctctagcggcccccggggaaga

SEQ ID NO: 204 - DNA, 90 bp, Homo sapiens
cgggagaagaaagccagtgcgtctctgggcgcaggggccagtggggctcggaggcacaggcaccccgcgacactccaggttccccgaccc

SEQ ID NO: 205 - DNA, 120 bp, Homo sapiens
gagcgcccgggctttgcggggagcaggctaaggcggccgagagaaaggggggtcgagacgggggggtggaggtttgggggggtgggggggcaggcggccgccatcttctcgccgaggccg

SEQ ID NO: 206 - DNA, 100 bp, Homo sapiens
ctgggaatccagctgtcgccgccccgcagagccccctgtccccggagggcgctcatttccgggccgcccaccacccgcgtagcaccggcagccgctgtcc

SEQ ID NO: 207 - DNA, 85 bp, Homo sapiens
gaggtctgcggcccaggttcgattcctgcgacttgtcctaggcaggcctgtatgtgcgcggcggccgcgtgctgtacagtgtgag

SEQ ID NO: 208 - DNA, 106 bp, Homo sapiens
ggagttggggctcacgagacggggcgtgcggggcaccgggcggccggcggggagtcgcaggcttccccagagggggcgcgagccgggccgccggggaactcaccat

SEQ ID NO: 209 - DNA, 114 bp, Homo sapiens
ggtgccctgacagaagtcgtcagccggtgtcatgcccaggacaggcatctgcagccttgtgtggacgtcaacgccaccaggaaggaccatcaggccgtgggcgtcaatggtctt

SEQ ID NO: 210 - DNA, 97 bp, Homo sapiens
ggggtcacagcctggcgctcggaccctggcccgggtcatctgcgaaggagccggctttggccaaggtgccttcctggacgggtgtggttcccagagc

SEQ ID NO: 211 - DNA, 89 bp, Homo sapiens
ggccccggctccgccctgcccgctgccctcggcgacggccgtggtccctgccctggggtcaattacagagcgaggtctgagccgcagag

SEQ ID NO: 212 - DNA, 113 bp, Homo sapiens
gggacagtggggctgacgcgtggcttcggcgccgcgcggtctcccgaatcccgagccccgcgcccggcgcggccggggtccccaaccgccctcccgctcgccgggacccccac

SEQ ID NO: 213 - DNA, 83 bp, Homo sapiens
gcgtgggcccccgccgcagctccgcggagcctcggtgtctcctgcaacagggggcggggggaacagcggcgagcagccctggg

SEQ ID NO: 214 - DNA, 120 bp, Homo sapiens
gggtgtccgacccaagccgagcccgagcccgagcccaggcaggagctttacagacagcctcttcccttcccacttcctgcaggcgccccacgcgtgcgatcctcccggccaagacccgcg

SEQ ID NO: 215 - DNA, 118 bp, Homo sapiens
gcccagccaagtccggcccgggggcccctaggctaggacatcgaggcaagagccacctgaaccgctggcgaattggtggctgctgcggcgacggcagcggaggttgcaaattgcaatc

SEQ ID NO: 216 - DNA, 113 bp, Homo sapiens
agggagcccagccaagtccggcccgggggcccctaggctaggacatcgaggcaagagccacctgaaccgctggcgaattggtggctgctgcggcgacggcagcggaggttgca

SEQ ID NO: 217 - DNA, 90 bp, Homo sapiens
cagagcggaggaagctgcggacctggggtgggggaacccgcccgcggacccctggcccccaccccgcgccggcctctgtgcccgcatctg

SEQ ID NO: 218 - DNA, 87 bp, Homo sapiens
tcggagtcccacagggcaacgaagcgcgggtagcggctgcggagccgggcggaggtgcgcggggccggggcgtgcggccagcaagag

SEQ ID NO: 219 - DNA, 111 bp, Homo sapiens
ctggagacctcgacagagcgccggccccctgactgcccgcgaagcgagacgcggggcgccgggtctagcgcagtgagcggcgaggcgcggcggaggtgcagccggcagccc

SEQ ID NO: 220 - DNA, 120 bp, Homo sapiens
gcgacaggcctccagcaagcaagcgcgggcggcatccgcagtctccagaagtttgagacttggccgtaagcggactcgtgcgccccaactctttgccgcgccagcgcctggagcggagag

SEQ ID NO: 221 - DNA, 91 bp, Homo sapiens
cccggggtgcagcggccgccggggctggggccggcgggagtccgcgggaccctccagaagagcggccggcgccgtgactcagcactggggc

SEQ ID NO: 222 - DNA, 101 bp, Homo sapiens
acgtaagagcctgggagcgcccgagccgcccggctgcccggagccccatcgcctaggaccgggagatgctggaaatgcaaccgcctgttccccgaggagcc

SEQ ID NO: 223 - DNA, 96 bp, Homo sapiens
gctccggggctcgctctccggcaggcccggggagaggtggggtgacaatgggttggggtgcgcgcgtgcctcataggtgcgagacagagcgagccg

SEQ ID NO: 224 - DNA, 97 bp, Homo sapiens
cctgccggcgccgccacccccgcacggctcgccctcgccgcgggcgcacatagggcagcagccgcacgcgtcgcgggtctcgcccagcaggcagccc

SEQ ID NO: 225 - DNA, 114 bp, Homo sapiens
cgcccatgggccggtcacgccgggtgcccgctcaccccccgacgccagcaggagcgcgcgcgcaggccccgcggggccgggagggcggcacgggcggggcccccgtgctctcac

SEQ ID NO: 226 - DNA, 83 bp, Homo sapiens
agctgagaaccggacggggtgggatcgaggagggtgcgaagcgccactgtttaggtttcgctttcccgggagcctgacccgcc

SEQ ID NO: 227 - DNA, 91 bp, Homo sapiens
gggactacaagccgcgccgcgctgccgctggcccctcagcaaccctcgacatggcgctgaggcggccaccgcgactccggctctgcgctcg

SEQ ID NO: 228 - DNA, 91 bp, Homo sapiens
gcgcgcagagctgcaaaggcccgagcaggagcacggtccaggcgaagcgcatcactccttttgccagattgaccccgctcgaggaggacgt

SEQ ID NO: 229 - DNA, 92 bp, Homo sapiens
ggcagaggcgaggagctgtccgccttgcacgtttccaatcgcattacgtgaacaaatagctgaggggcggccgggccagaacggcttgtgta

SEQ ID NO: 230 - DNA, 97 bp, Homo sapiens
gatgtcgtctgggagcagtgcgggcccctgcattgccaaggccttataggcacgggctgggcgggggtgggcagtccgccagccagcggcattctgc

SEQ ID NO: 231 - DNA, 109 bp, Homo sapiens
gcggggctccccctaccggccagacccggggagaggcgcgcggaggctgcgaaggttccagaagggcggggagggggcgccgcgcgctgaccctccctgggcaccgctg

SEQ ID NO: 232 - DNA, 97 bp, Homo sapiens
gccttcgctgccgccacgggcccgtcttcttcctccttcggctcccagggtaaggcgcggggcgcggggttggatgcaggcgccctgcccgctgcgc

SEQ ID NO: 233 - DNA, 113 bp, Homo sapiens
agaattcaggccggcctctatcgcttcccagaacgattgcaccactgccgctgccgccggcctgacactgcctcagcctcagtgctggcagctttgggagaagaaccctgcgc

SEQ ID NO: 234 - DNA, 82 bp, Homo sapiens
gccggatgaagcattcgggcgttcccactgcggaagggcggggatggctgtgacgcaggcgtgcccgccgtcgcctgcaggg

SEQ ID NO: 235 - DNA, 96 bp, Homo sapiens
ccactccgcccccagggagctcccgggtcatcctctcatccgggctgccccgcggcccccaaggagccccacccccgggaccaaatggcccgcaag

SEQ ID NO: 236 - DNA, 109 bp, Homo sapiens
ccccgtgtgggaagagctcgtctgggtgcagcgccgcggcccgcaacattagcaacggcagcagcagtagcactggtaacgacgacagcaccggccgcccggccacccg

SEQ ID NO: 237 - DNA, 97 bp, Homo sapiens
ccttggtcaccaggtagcccggctcggcggcccgcccggggcatcagctcggtgcagggcgcggagccgttctgcagcgggaacagcacgaagagtg

SEQ ID NO: 238 - DNA, 114 bp, Homo sapiens
tcactctgcgagcggccccgcgacacgaggcagccgctcccgtcctccgacgcggccatgggcccggccggcgcgggggcaagttagagcgagccgcgtggaatcagagcatcc

SEQ ID NO: 239 - DNA, 75 bp, Homo sapiens
gacctgggcgcctctggctctccgcggtcccgagttctcgacaaactttctgcgccgactgcggcatgagaagcc

SEQ ID NO: 240 - DNA, 113 bp, Homo sapiens
gggcgctgaggcagaagaacgtgcacgaggtgaaggaccacaaattcatcgcgcgcttcttcaagcagcccaccttctgcagccactgcaccgacttcatctggtaggtgccg

SEQ ID NO: 241 - DNA, 102 bp, Homo sapiens
ccaggccggaggccccgcccccttcctcctggctcctcccctcctccgtgggccggccgccaacgacgccagagccggaaatgacgacaacggtgagggttc

SEQ ID NO: 242 - DNA, 103 bp, Homo sapiens
gatgctccggccgaggtcccgccgcagccctcccccagcccccaggtcgcggcgccctcacccgggacccctccggacctggcgcatccatctgcagccgccc

SEQ ID NO: 243 - DNA, 126 bp, Homo sapiens
tgcagcctccggagtcagtgccgcgcgcccgccgccccgcgccttcctgctcgccgcacctccgggagccggggcgcacccagcccgcagcgccgcctccccgcccgcgccgcctccgaccgcagg

SEQ ID NO: 244 - DNA, 96 bp, Homo sapiens
tcctgctcgccgcacctccgggagccggggcgcacccagcccgcagcgccgcctccccgcccgcgccgcctccgaccgcaggccgagggccgccac

SEQ ID NO: 245 - DNA, 80 bp, Homo sapiens
agtacagagtggggagcgcagcgacgaagaatgaacagggcctcgtgaggtcccaaacacccgtttcggtggcaggaaag

SEQ ID NO: 246 - DNA, 79 bp, Homo sapiens
ctgcgccagccgcttgcgctcgtccttagcccacaccatgaaagcgttcatcggccgccggatacgggactcgcccttg

SEQ ID NO: 247 - DNA, 79 bp, Homo sapiens
gccagccgagtccggaggcatcgggaggtcgagagccgccgggaccccagctctgcgttcactgccccgtccggagctg

SEQ ID NO: 248 - DNA, 95 bp, Homo sapiens
gcgccggaggccaaggttgccccgcacggcccggcgggcgagcgagctcgggctgcagcagccccgccggcggcgcgcacggcaactttggagag

SEQ ID NO: 249 - DNA, 99 bp, Homo sapiens
cgggtcagagcactgtccggtggtgcccaggaggagtaggagcaggagcagaagcagaagcggggtccggagctgcgcgcctacgcgggacctgtgtcc

SEQ ID NO: 250 - DNA, 114 bp, Homo sapiens
gacctcaccggggaaacaccgcggacagtcgggccagcagcgcccggagctcactccaggtctccaaacttgcagcacttcccagagcgcgcgcgctcggagcgggacctgctt

SEQ ID NO: 251 - DNA, 117 bp, Homo sapiens
ttaccccgcaggctgaggccagggcgtggcggctgctgggatcccggagcttctcagtagcaggggctgcgggaggaagtgaagccgggaggggctgccggcgctgacagcagagga

SEQ ID NO: 252 - DNA, 108 bp, Homo sapiens
gctgcatcttcgcggagcgcccggcagaaatagcctagggaagacgaaaaacagctagcggagccgcccaggctgcagctataaagcgccggccagacgcactgtgag

SEQ ID NO: 253 - DNA, 80 bp, Homo sapiens
gcccaggctgtggcctaggccgtcggttcccggccatgcctagctcctctgaggtcgcccttagtgaggacacgaggtgc

SEQ ID NO: 254 - DNA, 97 bp, Homo sapiens
ggggcccccaggcacccggcccgcacgcaaaccctcagcccggggccccctgacccccgcgttcacccctcagcccggccgccgggcactgcgcatc

SEQ ID NO: 255 - DNA, 95 bp, Homo sapiens
ccggccttcagtcccctcggccgcggggtgggagctgggggctgggccggcagccgggaccccgggcgtcctgtcccgtttctgcgcagcaactg

SEQ ID NO: 256 - DNA, 116 bp, Homo sapiens
ccaccgagaagcgccggcctcggggctgtctacagcggcccgggagaggctgtggtggccccgagcgcgagtgtgtaggtgacaggacagcggccaggcccgcccctcccctcggt

SEQ ID NO: 257 - DNA, 23 bp, Homo sapiens
tcggagttttatagggtaacgaa

SEQ ID NO: 258 - DNA, 21 bp, Homo sapiens
ttggagatttcgatagagcgt

SEQ ID NO: 259 - DNA, 23 bp, Homo sapiens
ttaaataaaaggtttacgagcgg

SEQ ID NO: 260 - DNA, 23 bp, Homo sapiens
cgtaagtaatgtttcgagcgagt

SEQ ID NO: 261 - DNA, 24 bp, Homo sapiens
gttttcgtaagtaatgtttcgagc

SEQ ID NO: 262 - DNA, 21 bp, Homo sapiens
ttcggggtattgtttacgaag

SEQ ID NO: 263 - DNA, 22 bp, Homo sapiens
gtcgtaataggttcggttcgtt

SEQ ID NO: 264 - DNA, 19 bp, Homo sapiens
ctcttactaaccgcacgcc

SEQ ID NO: 265 - DNA, 21 bp, Homo sapiens
aaactaccgactacacctccg

SEQ ID NO: 266 - DNA, 22 bp, Homo sapiens
taaatatccgacgacaaacgaa

SEQ ID NO: 267 - DNA, 21 bp, Homo sapiens
tacttccacgtctacctcccg

SEQ ID NO: 268 - DNA, 21 bp, Homo sapiens
tacttccacgtctacctcccg

SEQ ID NO: 269 - DNA, 23 bp, Homo sapiens
aataaaatctaaaaccgcacacg

SEQ ID NO: 270 - DNA, 21 bp, Homo sapiens
aacacgtcctactatcctcgc

SEQ ID NO: 271 - DNA, 87 bp, Homo sapiens; identical to SEQ ID NO: 154

SEQ ID NO: 272 - DNA, 111 bp, Homo sapiens; identical to SEQ ID NO: 155

SEQ ID NO: 273 - DNA, 103 bp, Homo sapiens
ttaaataaaaggtttacgagcggcggtttcgacgtggggagtaagtaggtttttttggtttttgtagtaggcggatttaggttcgtttgtcgtcggatattta

SEQ ID NO: 274 - DNA, 95 bp, Homo sapiens
cgtaagtaatgtttcgagcgagtagattttaggataataaataaattaaaattatattttaaattattggagatcgggaggtagacgtggaagta

SEQ ID NO: 275 - DNA, 100 bp, Homo sapiens
gttttcgtaagtaatgtttcgagcgagtagattttaggataataaataaattaaaattatattttaaattattggagatcgggaggtagacgtggaagta

SEQ ID NO: 276 - DNA, 113 bp, Homo sapiens
ttcggggtattgtttacgaagatcgtttatatgttgttgtatttttttatttatttgttacgtgatcgcgtttgtttattataggtttaacgtgtgcggttttagattttatt

SEQ ID NO: 277 - DNA, 120 bp, Homo sapiens
gtcgtaataggttcggttcgttatagtaggttttgaaggcgggtttttagcgttcgagtatcgcgaggagggtgtcgtagtggttagtcgcgttcgtcggcgaggatagtaggacgtgtt

SEQ ID NO: 278 - DNA, 87 bp, Homo sapiens; identical to SEQ ID NO: 218

SEQ ID NO: 279 - DNA, 111 bp, Homo sapiens; identical to SEQ ID NO: 219

SEQ ID NO: 280 - DNA, 103 bp, Homo sapiens
tcaaacaaaaggctcacgagcggcggccccgacgtggggagcaagcaggcttctctggcccctgcagcaggcggacccaggcccgcctgccgccggacattca

SEQ ID NO: 281 - DNA, 95 bp, Homo sapiens
cgtaagcaatgccccgagcgagtagatttcaggacaacaaacaaatcaaaattacacttcaaattattggagatcgggaggcagacgtggaagca

SEQ ID NO: 282 - DNA, 100 bp, Homo sapiens
gtcttcgtaagcaatgccccgagcgagtagatttcaggacaacaaacaaatcaaaattacacttcaaattattggagatcgggaggcagacgtggaagca

SEQ ID NO: 283 - DNA, 113 bp, Homo sapiens
ctcggggcattgcttacgaagaccgtttatatgttgctgcatccctctacctatctgttacgtgaccgcgcttgtccatcacaggcccaacgtgtgcggctccagattccact

SEQ ID NO: 284 - DNA, 120 bp, Homo sapiens
gccgcaacaggttcggtccgctacagcaggctctgaaggcgggtttctagcgcccgagtatcgcgaggagggtgccgcagtggccagccgcgtccgccggcgaggacagcaggacgtgct

SEQ ID NO: 285 - DNA, 19 bp, Homo sapiens
gtagtggcgagatgacgga

SEQ ID NO: 286 - DNA, 23 bp, Homo sapiens
agtgtttagagagttcgtcggtt

SEQ ID NO: 287 - DNA, 21 bp, Homo sapiens
gtcgggtgttatatggtgcgt

SEQ ID NO: 288 - DNA, 22 bp, Homo sapiens
ttttgagaggtcgttatcgtgt

SEQ ID NO: 289 - DNA, 19 bp, Homo sapiens
cgttaggtagggaggaggc

SEQ ID NO: 290 - DNA, 22 bp, Homo sapiens
atttgggtagaaaagttcgttc

SEQ ID NO: 291 - DNA, 22 bp, Homo sapiens
aacgatttggggttagagtttc

SEQ ID NO: 292 - DNA, 19 bp, Homo sapiens
ttttcgttatggacgcgga

SEQ ID NO: 293 - DNA, 25 bp, Homo sapiens
cgttcgtatagagttcggtaatgtc

SEQ ID NO: 294 - DNA, 21 bp, Homo sapiens
cgtttttggttttgttttcgt

SEQ ID NO: 295 - DNA, 21 bp, Homo sapiens
tttagtaggtttggtcgaggc

SEQ ID NO: 296 - DNA, 21 bp, Homo sapiens
tataggtaagggggcggtttc

SEQ ID NO: 297 - DNA, 18 bp, Homo sapiens
gcgttgaagtcggggttc

SEQ ID NO: 298 - DNA, 19 bp, Homo sapiens
ttagaggggcgtagggtgc

SEQ ID NO: 299 - DNA, 21 bp, Homo sapiens
cgttgtaggattttgggggtc

SEQ ID NO: 300 - DNA, 19 bp, Homo sapiens
aaccgaaaccaaacaaacg

SEQ ID NO: 301 - DNA, 22 bp, Homo sapiens
cgtaacgaataaactacgcgat

SEQ ID NO: 302 - DNA, 21 bp, Homo sapiens
aaaaacaatcctcgtccgaaa

SEQ ID NO: 303 - DNA, 22 bp, Homo sapiens
ttactcgaactattccccgatt

SEQ ID NO: 304 - DNA, 24 bp, Homo sapiens
aacgataaaataaaaacaacgacc

SEQ ID NO: 305 - DNA, 21 bp, Homo sapiens
ctcaataattttcccgacgac

SEQ ID NO: 306 - DNA, 22 bp, Homo sapiens
taaccaaactaccgctttacga

SEQ ID NO: 307 - DNA, 20 bp, Homo sapiens
ataaccttaccgaccccgaa

SEQ ID NO: 308 - DNA, 22 bp, Homo sapiens
cctataactaaacgcgacgcac

SEQ ID NO: 309 - DNA, 19 bp, Homo sapiens
aacctaattcccgcccgtt

SEQ ID NO: 310 - DNA, 19 bp, Homo sapiens
acgctaaccccaaaatccg

SEQ ID NO: 311 - DNA, 20 bp, Homo sapiens
gcgactctaaaaattccgct

SEQ ID NO: 312 - DNA, 24 bp, Homo sapiens
cccgtacttcgctaactttaaacg

SEQ ID NO: 313 - DNA, 21 bp, Homo sapiens
gccaaactaaaatcccaacga

SEQ ID NO: 314 - DNA, 19 bp, Homo sapiens
acccttctcccgctcgata

SEQ ID NO: 315 - DNA, 92 bp, Homo sapiens
gtagtggcgagatgacggatatttagcgagtttaatgggcgtcgaacgcgtttaggtttggtggatttgttagcgtttgtttggtttcggtt

SEQ ID NO: 316 - DNA, 94 bp, Homo sapiens
agtgtttagagagttcgtcggttttatcgtttttttaaaggagaattcggtttatcgttcgtcgcggcggcgatcgcgtagtttattcgttacg

SEQ ID NO: 317 - DNA, 116 bp, Homo sapiens
gtcgggtgttatatggtgcgtgataatttgtttttgatttgggtttatttgaagagcgtagaattttaataaataaatagttttttgggatttgttttcggacgaggattgttttt

SEQ ID NO: 318 - DNA, 107 bp, Homo sapiens
ttttgagaggtcgttatcgtgttatgggcgtgcgtaattgtttttacggtaataatatgttaggataacgcgatattttttttgaaatcggggaatagttcgagtaa

SEQ ID NO: 319 - DNA, 117 bp, Homo sapiens
cgttaggtagggaggaggcggggaggggttggttttagaagtgcgtgtttgaagcggttaatgtgtgtaaattagtaaggaggaggggtgcggggtcgttgtttttattttatcgtt

SEQ ID NO: 320 - DNA, 87 bp, Homo sapiens
atttgggtagaaaagttcgttcgtgacgttattaagtttcggaagttttttggcgtcggcgtaagggtcgtcgggaaaattattgag

SEQ ID NO: 321 - DNA, 109 bp, Homo sapiens
aacgatttggggttagagtttcgggagttggcggagcgcggagttcgtatcgtttttagaggtaggacgtagtttttttttttgaattcgtaaagcggtagtttggtta

SEQ ID NO: 322 - DNA, 88 bp, Homo sapiens
ttttcgttatggacgcggacgattttcgggtttttaagggttttttgcggaagtttttggagtattttttcggggtcggtaaggttat

SEQ ID NO: 323 - DNA, 108 bp, Homo sapiens
cgttcgtatagagttcggtaatgtcgaggtttttttaacgggtcggtttgcgaggagtaaaaaaggggtttagaggagggtagcgcgtgcgtcgcgtttagttatagg

SEQ ID NO: 324 - DNA, 119 bp, Homo sapiens
cgtttttggttttgttttcgtcgcggagcggaattttttaagtcgcggtttgaggaggaaggaaaagggggcggttcgggagagtcgttgcgaaattagtaacgggcgggaattaggtt

SEQ ID NO: 325 - DNA, 120 bp, Homo sapiens
tttagtaggtttggtcgaggcgggattttcgtcgttttagttttcgttttcgttttcgcgttttttcgtcgcgcggagttttggttggtttgttttttaatcggattttggggttagcgt

SEQ ID NO: 326 - DNA, 109 bp, Homo sapiens
tataggtaagggggcggtttcgtttcgcgttttggaacgattttacggtttcgtttatattttcgtttttggttttattttcgtcgtagagcggaatttttagagtcgc

SEQ ID NO: 327 - DNA, 75 bp, Homo sapiens
gcgttgaagtcggggttcgttttgtggtttcgttcggttcgcgtttgttagcgtttaaagttagcgaagtacggg

SEQ ID NO: 328 - DNA, 113 bp, Homo sapiens
ttagaggggcgtagggtgcgcgggggtcgttggtttttcgggtattttttttttgcggttttttcgttttttttcggagttggtgtttgaggtcgttgggattttagtttggc

SEQ ID NO: 329 - DNA, 84 bp, Homo sapiens
cgttgtaggattttgggggtcggacggtgggatacggttaattttcggggagatgttgtggtttttatcgagcgggagaagggt

SEQ ID NO: 330 - DNA, 92 bp, Homo sapiens
gcagtggcgagatgacggacacccagcgagtccaatgggcgtcgaacgcgtctaggcttggtggacttgtcagcgcctgcctggcttcggtc

SEQ ID NO: 331 - DNA, 94 bp, Homo sapiens
agtgcccagagagtccgccggtcccaccgccccttcaaaggagaacccggcccaccgcccgccgcggcggcgaccgcgcagcccactcgtcacg

SEQ ID NO: 332 - DNA, 116 bp, Homo sapiens
gccgggtgtcacatggtgcgtgataacttgcccttgatttgggttcatttgaagagcgtagaactctaacaaataaacagccttttgggacctgtccccggacgaggactgccccc

SEQ ID NO: 333 - DNA, 107 bp, Homo sapiens
ttttgagaggccgccaccgtgttatgggcgtgcgcaactgcctctacggcaataatatgtcaggacaacgcgatatcccccctgaaatcggggaacagcccgagcaa

SEQ ID NO: 334 - DNA, 117 bp, Homo sapiens
cgccaggcagggaggaggcggggaggggctggccccagaagtgcgtgtctgaagcggccaatgtgtgcaaatcagcaaggaggaggggtgcggggccgctgcccccacctcaccgcc

SEQ ID NO: 335 - DNA, 87 bp, Homo sapiens
atttgggtagaaaagctcgctcgtgacgtcaccaagctccggaagtctcctggcgtcggcgcaagggccgccgggaaaaccattgag

SEQ ID NO: 336 - DNA, 109 bp, Homo sapiens
aacgacctggggctagagccccgggagctggcggagcgcggagtccgcatcgtctccagaggtaggacgcagcttttctctctgaatccgcaaagcggcagcttggtca

SEQ ID NO: 337 - DNA, 88 bp, Homo sapiens
tcctcgccatggacgcggacgactcccgggcccccaagggctccttgcggaagttcctggagcacctctccggggccggcaaggccat

SEQ ID NO: 338 - DNA, 108 bp, Homo sapiens
cgctcgcacagagcccggcaatgccgaggccctcccaacgggtcggtctgcgaggagcaaaaaaggggttcagaggagggcagcgcgtgcgtcgcgctcagctatagg

SEQ ID NO: 339 - DNA, 120 bp, Homo sapiens
cccagcaggcctggccgaggcgggaccttcgtcgctccagcccccgtccccgcccccgcgcctccccgccgcgcggagctctggttggcttgctttccaaccggactttggggctagcgt

SEQ ID NO: 340 - DNA, 120 bp, Homo sapiens; identical to SEQ ID NO: 339

SEQ ID NO: 341 - DNA, 109 bp, Homo sapiens
cacaggtaagggggcggccccgccccgcgccctggaacgacctcacggccccgcccacatccccgcccctggccccacctccgccgcagagcggaaccctcagagtcgc

SEQ ID NO: 342 - DNA, 75 bp, Homo sapiens
gcgctgaagtcggggcccgccctgtggccccgcccggcccgcgcttgctagcgcccaaagccagcgaagcacggg

SEQ ID NO: 343 - DNA, 113 bp, Homo sapiens
tcagaggggcgcagggtgcgcgggggccgttggccctccgggcacttcccctttgcggtctccccgccctccttcggagctggtgcctgaggtcgctgggacctcagcctggc

SEQ ID NO: 344 - DNA, 84 bp, Homo sapiens
cgctgcaggaccctgggggccggacggtgggatacggccaatctccggggagatgctgtggctcttaccgagcgggagaagggt

SEQ ID NO: 345 - DNA, 27 bp, Homo sapiens
ttatatgtcggttacgtgcgtttatat

SEQ ID NO: 346 - DNA, 23 bp, Homo sapiens
agcgtagagataggttggtaacg

SEQ ID NO: 347 - DNA, 21 bp, Homo sapiens
tacgcgtaggttttaagtcgc

SEQ ID NO: 348 - DNA, 25 bp, Homo sapiens
aaagttagtaggagtaagaggacgc

SEQ ID NO: 349 - DNA, 20 bp, Homo sapiens
ggggttatagtttggcgttc

SEQ ID NO: 350 - DNA, 20 bp, Homo sapiens
ttcggggtgtagcggtcgtc

SEQ ID NO: 351 - DNA, 19 bp, Homo sapiens
gtttcggggttcgtttttc

SEQ ID NO: 352 - DNA, 19 bp, Homo sapiens
agttttcggagaagacggc

SEQ ID NO: 353 - DNA, 32 bp, Homo sapiens
cgttaggttattttttaaatagagtcggtagc

SEQ ID NO: 354 - DNA, 20 bp, Homo sapiens
gggattataagtcgcgtcgc

SEQ ID NO: 355 - DNA, 22 bp, Homo sapiens
cgtgaataaatagttgaggggc

SEQ ID NO: 356 - DNA, 27 bp, Homo sapiens
gcggggttttttttatcggttagattc

SEQ ID NO: 357 - DNA, 25 bp, Homo sapiens
gcggtttttaaggagttttattttc

SEQ ID NO: 358 - DNA, 22 bp, Homo sapiens
aacgatttggggttagagtttc

SEQ ID NO: 359 - DNA, 25 bp, Homo sapiens
cgttcgtatagagttcggtaatgtc

SEQ ID NO: 360 - DNA, 23 bp, Homo sapiens
tcgtcgttttagttttcgttttc

SEQ ID NO: 361 - DNA, 18 bp, Homo sapiens
gcgttgaagtcggggttc

SEQ ID NO: 362 - DNA, 21 bp, Homo sapiens
aggtaggttttagttttcggc

SEQ ID NO: 363 - DNA, 26 bp, Homo sapiens
tgtagttttcggagttagtgtcgcgc

SEQ ID NO: 364 - DNA, 19 bp, Homo sapiens
gagatgtttcgagggttgc

SEQ ID NO: 365 - DNA, 22 bp, Homo sapiens
gttagtcgagttcggaggtatc

SEQ ID NO: 366 - DNA, 19 bp, Homo sapiens
gaaccaaaacgctccccat

SEQ ID NO: 367 - DNA, 22 bp, Homo sapiens
aaaacgataacccttaaaccga

SEQ ID NO: 368 - DNA, 22 bp, Homo sapiens
tcccgaactaaacgaaaccccg

SEQ ID NO: 369 - DNA, 19 bp, Homo sapiens
aatacgacgccaaaaaccg

SEQ ID NO: 370 - DNA, 21 bp, Homo sapiens
gctctaaaaaccacacccgtc

SEQ ID NO: 371 - DNA, 22 bp, Homo sapiens
gccccaatactaaatcacgacg

SEQ ID NO: 372 - DNA, 20 bp, Homo sapiens
cgactcgctctatctcgcac

SEQ ID NO: 373 - DNA, 26 bp, Homo sapiens
acgactaaactactataaccctacga

SEQ ID NO: 374 - DNA, 27 bp, Homo sapiens
cgactatacgaactaacgaataaaccg

SEQ ID NO: 375 - DNA, 20 bp, Homo sapiens
cgaacgcaaaaccgaaatcg

SEQ ID NO: 376 - DNA, 20 bp, Homo sapiens
gacaatcccgaaaaacgaac

SEQ ID NO: 377 - DNA, 27 bp, Homo sapiens
caacgatacccaaaaaaaatcaacgcg

SEQ ID NO: 378 - DNA, 21 bp, Homo sapiens
ctacgaaattccctttacgct

SEQ ID NO: 379 - DNA, 22 bp, Homo sapiens
taaccaaactaccgctttacga

SEQ ID NO: 380 - DNA, 22 bp, Homo sapiens
cctataactaaacgcgacgcac

SEQ ID NO: 381 - DNA, 19 bp, Homo sapiens
acgctaaccccaaaatccg

SEQ ID NO: 382 - DNA, 24 bp, Homo sapiens
cccgtacttcgctaactttaaacg

SEQ ID NO: 383 - DNA, 20 bp, Homo sapiens
gacctcaaacaccaactccg

SEQ ID NO: 384 - DNA, 25 bp, Homo sapiens
cctacgatcgaaaacgacgcgaacg

SEQ ID NO: 385 - DNA, 20 bp, Homo sapiens
ccgcaatatcactaaaccga

SEQ ID NO: 386 - DNA, 24 bp, Homo sapiens
caactccgaacgaaacaataaacg

SEQ ID NO: 387 - DNA, 38 bp, Homo sapiens
cgtctgccccgtcgaaaacccgccgattaacgcagacg

SEQ ID NO: 388 - DNA, 36 bp, Homo sapiens
cgacatgcgcggaggggttagcgtggttgcatgtcg

SEQ ID NO: 389 - DNA, 37 bp, Homo sapiens
cgacatgcctacgaccgcgtcgcccattagcatgtcg

SEQ ID NO: 390 - DNA, 32 bp, Homo sapiens
cgacatgcgggcggtcggggtcgcgcatgtcg

SEQ ID NO: 391 - DNA, 42 bp, Homo sapiens
cgacatgcggttcgggttatttgcgaaggagtcggcatgtcg

SEQ ID NO: 392 - DNA, 41 bp, Homo sapiens
cgtctgcttggggtcggcgggagttcgcgggattgcagacg

SEQ ID NO: 393 - DNA, 35 bp, Homo sapiens
cgacatgcacgcgcgcaccccaacccagcatgtcg

SEQ ID NO: 394 - DNA, 38 bp, Homo sapiens
cgacatgcaccacgaccacgcgaatcgaacgcatgtcg

SEQ ID NO: 395 - DNA, 37 bp, Homo sapiens
cgacatgcacgccgaccgcgatctaactcgcatgtcg

SEQ ID NO: 396 - DNA, 35 bp, Homo sapiens
cgacacgatatggcgttgaggcggttatcgtgtcg

SEQ ID NO: 397 - DNA, 38 bp, Homo sapiens
cgtctgccacaaaccgttctaacccgaccgcgcagacg

SEQ ID NO: 398 - DNA, 32 bp, Homo sapiens
cgacatgccgacgccccctccccggcatgtcg

SEQ ID NO: 399 - DNA, 39 bp, Homo sapiens
cgacatgggttcgtaaggtttggggtagcggccatgtcg

SEQ ID NO: 400 - DNA, 32 bp, Homo sapiens
cgacatgcgcgctccgccaactccgcatgtcg

SEQ ID NO: 401 - DNA, 38 bp, Homo sapiens
cgacatgcggggtttagaggagggtagcgcgcatgtcg

SEQ ID NO: 402 - DNA, 27 bp, Homo sapiens
actccgcgttttttcgtcgcgcggagt

SEQ ID NO: 403 - DNA, 42 bp, Homo sapiens
cgtctgcgtggtttcgttcggttcgcgtttgttaggcagacg

SEQ ID NO: 404 - DNA, 36 bp, Homo sapiens
cgacatgcgggtgcgcgggggtcgttgggcatgtcg

SEQ ID NO: 405 - DNA, 47 bp, Homo sapiens
cgacatgctcgggagtcggggcgtatttagttcgtagcggcatgtcg

SEQ ID NO: 406 - DNA, 43 bp, Homo sapiens
cgacatgcgttcgtgttttggtttgtcgcggtttggcatgtcg

SEQ ID NO: 407 - DNA, 36 bp, Homo sapiens
cgacatgcccgacgactctcgacctcccgcatgtcg

SEQ ID NO: 408 - DNA, 74 bp, Homo sapiens
ttatatgtcggttacgtgcgtttatatttagttaatcggcgggttttcgacgggaatggggagcgttttggttc

SEQ ID NO: 409 - DNA, 111 bp, Homo sapiens
agcgtagagataggttggtaacggttttagggaggcgcggaggggttagcgtggttggtttaaaaggatatagggattgaggggtaagatcggtttaagggttatcgtttt

SEQ ID NO: 410 - DNA, 113 bp, Homo sapiens
tacgcgtaggttttaagtcgcggttaatgggcgacgcggtcgtagattcgttcggtttcgttttgttttgtgagttttttcggtcgggttgcggggtttcgtttagttcggga

SEQ ID NO: 411 - DNA, 117 bp, Homo sapiens
aaagttagtaggagtaagaggacgcgtaggagggtttcggtcgcggttatttttgggcggtcggggtcgcggtttcgggagcggtgcgggcgcgggttcggtttttggcgtcgtatt

SEQ ID NO: 412 - DNA, 97 bp, Homo sapiens; identical to SEQ ID NO: 146

SEQ ID NO: 413 - DNA, 91 bp, Homo sapiens; identical to SEQ ID NO: 157

SEQ ID NO: 414 - DNA, 96 bp, Homo sapiens; identical to SEQ ID NO: 159

SEQ ID NO: 415 - DNA, 101 bp, Homo sapiens
agttttcggagaagacggcgtttttaacgttcgattcgcgtggtcgtggtagcgttacgcgagttttttaggcgatcgtagggttatagtagtttagtcgt

SEQ ID NO: 416 - DNA, 117 bp, Homo sapiens
cgttaggttattttttaaatagagtcggtagcgcgtttcgttcggtatttttcgaagagttagatcgcggtcggcgttagcgttatcgttcggtttattcgttagttcgtatagtcg

SEQ ID NO: 417 - DNA, 91 bp, Homo sapiens; identical to SEQ ID NO: 163

SEQ ID NO: 418 - DNA, 124 bp, Homo sapiens
cgtgaataaatagttgaggggcggtcgggttagaacggtttgtgtaattttgtaaacgtgttagaaagtttaaaatttttttttttttttttattttagatattgttcgtttttcgggattgtc

SEQ ID NO: 419 - DNA, 109 bp, Homo sapiens; identical to SEQ ID NO: 167

SEQ ID NO: 420 - DNA, 99 bp, Homo sapiens
gcggtttttaaggagttttattttcgggattaaatggttcgtaaggtttggggtagcggcgttgtaggagatgagtttagcgtaaagggaatttcgtag

SEQ ID NO: 421 - DNA, 109 bp, Homo sapiens; identical to SEQ ID NO: 321

SEQ ID NO: 422 - DNA, 108 bp, Homo sapiens; identical to SEQ ID NO: 323

SEQ ID NO: 423 - DNA, 92 bp, Homo sapiens
tcgtcgttttagttttcgttttcgttttcgcgttttttcgtcgcgcggagttttggttggtttgttttttaatcggattttggggttagcgt

SEQ ID NO: 424 - DNA, 75 bp, Homo sapiens; identical to SEQ ID NO: 327

SEQ ID NO: 425 - DNA, 133 bp, Homo sapiens
aggtaggttttagttttcggcgcggggaggcggcgcgttttagaggggcgtagggtgcgcgggggtcgttggtttttcgggtattttttttttgcggttttttcgttttttttcggagttggtgtttgaggtc

SEQ ID NO: 426 - DNA, 126 bp, Homo sapiens; identical to SEQ ID NO: 179

SEQ ID NO: 427 - DNA, 117 bp, Homo sapiens
gagatgtttcgagggttgcgcgggtttttcggttcgaagtcgtcgttcgtgttttggtttgtcgcggtttggtttatagcgtatttagggtttttagtcggtttagtgatattgcgg

SEQ ID NO: 428 - DNA, 79 bp, Homo sapiens; identical to SEQ ID NO: 183

SEQ ID NO: 429 - DNA, 74 bp, Homo sapiens
ccacatgtcggtcacgtgcgcccacacccagccaatcggcgggctcccgacgggaatggggagcgccctggtcc

SEQ ID NO: 430 - DNA, 111 bp, Homo sapiens
agcgcagagacaggctggcaacggcttcagggaggcgcggaggggtcagcgtggctggcttaaaaggatacagggactgaggggcaagaccggctcaagggtcaccgcttc

SEQ ID NO: 431 - DNA, 113 bp, Homo sapiens
cacgcgcaggccccaagtcgcggccaatgggcgacgcggccgcagatccgcccggccccgccctgccctgtgagttcctccggccgggctgcggggctccgctcagtccggga

SEQ ID NO: 432 - DNA, 117 bp, Homo sapiens
aaagccagcaggagcaagaggacgcgcaggagggcttcggtcgcggtcatctctgggcggccggggtcgcggtcccgggagcggtgcgggcgcgggtccggctcctggcgccgcact

SEQ ID NO: 433 - DNA, 97 bp, Homo sapiens; identical to SEQ ID NO: 210

SEQ ID NO: 434 - DNA, 91 bp, Homo sapiens; identical to SEQ ID NO: 221

SEQ ID NO: 435 - DNA, 96 bp, Homo sapiens; identical to SEQ ID NO: 223

SEQ ID NO: 436 - DNA, 101 bp, Homo sapiens
agcccccggagaagacggcgcccccaacgcccgacccgcgtggccgtggcagcgccacgcgagccctctaggcgaccgcagggccacagcagctcagccgc

SEQ ID NO: 437 - DNA, 117 bp, Homo sapiens
cgtcaggccactccttaaatagagccggcagcgcgctccgctcggcatttcccgaagagccagatcgcggccggcgccagcgccaccgtccggtccacccgccagcccgcacagccg

SEQ ID NO: 438 - DNA, 91 bp, Homo sapiens; identical to SEQ ID NO: 227

SEQ ID NO: 439 - DNA, 124 bp, Homo sapiens
cgtgaacaaatagctgaggggcggccgggccagaacggcttgtgtaactttgcaaacgtgccagaaagtttaaaatctctcctccttccttcactccagacactgcccgctctccgggactgcc

SEQ ID NO: 440 - DNA, 109 bp, Homo sapiens; identical to SEQ ID NO: 231

SEQ ID NO: 441 - DNA, 99 bp, Homo sapiens
gcggcccccaaggagccccacccccgggaccaaatggcccgcaaggtttggggcagcggcgttgcaggagatgagctcagcgcaaagggaaccccgcag

SEQ ID NO: 442 - DNA, 109 bp, Homo sapiens; identical to SEQ ID NO: 336

SEQ ID NO: 443 - DNA, 108 bp, Homo sapiens; identical to SEQ ID NO: 338

SEQ ID NO: 444 - DNA, 92 bp, Homo sapiens
tcgtcgctccagcccccgtccccgcccccgcgcctccccgccgcgcggagctctggttggcttgctttccaaccggactttggggctagcgt

SEQ ID NO: 445 - DNA, 75 bp, Homo sapiens; identical to SEQ ID NO: 342

SEQ ID NO: 446 - DNA, 119 bp, Homo sapiens
aggcaggtcccagtccccggcgcggggaggcggcgcgcttcagaggggcgcagggtgcgcgggggccgttggccctccgggcacttcccctttgcggtctccccgccctccttcggagc

SEQ ID NO: 447 - DNA, 126 bp, Homo sapiens; identical to SEQ ID NO: 243

SEQ ID NO: 448 - DNA, 117 bp, Homo sapiens
gagatgccccgagggctgcgcgggtctcccggcccgaagccgccgcccgtgttctggcctgtcgcggtctggtctacagcgtacccagggcccccagccggcctagtgacactgcgg

SEQ ID NO: 449 - DNA, 79 bp, Homo sapiens; identical to SEQ ID NO: 247